(19) United States
(12) Patent Application Publication
(10) Pub. No.: US 2011/0195448 A1
(43) Pub. Date: Aug. 11, 2011

(54) Recombinant Thraustochytrids that Grow on Xylose, and Compositions, Methods of Making, and Uses Thereof

(76) Inventors: James Casey LIPPMEIER, Columbia, MD (US); Kirk Emil Apt, Ellicott City, MD (US)

(21) Appl. No.: 12/980,321

(22) Filed: Dec. 28, 2010

Related U.S. Application Data
(60) Provisional application No. 61/290,471, filed on Dec. 28, 2009.

(51) Int. Cl. C12P 1/00; C12N 15/63; C12N 1/13

(52) U.S. Cl. 435/41; 435/471; 435/257.2

(57) ABSTRACT

The present invention is directed to recombinant thraustochytrids that grow on xylose and cell cultures comprising the recombinant thraustochytrids as well as methods of producing cell cultures, biomasses, microbial oils, compositions, and biofuels using the recombinant thraustochytrids.

### CROSS-REFERENCE TO RELATED APPLICATIONS

This application claims the benefit of the filing date of U.S. Appl. No. 61/290,471, filed Dec. 28, 2009, which is hereby incorporated by reference herein in its entirety.

### REFERENCE TO SEQUENCE LISTING SUBMITTED ELECTRONICALLY

The content of the electronically submitted sequence listing (“sequencelisting_ascii.txt”, 126,767 bytes, created on Dec. 20, 2010) filed with the application is incorporated herein by reference in its entirety.

### BACKGROUND OF THE INVENTION

1. Field of the Invention

The present invention is directed to recombinant thraustochytrids that grow on xylose and cell cultures comprising the recombinant thraustochytrids, as well as methods of producing cell cultures, biomass, microbial oils, compositions, and biofuels using the recombinant thraustochytrids.

2. Background

Fatty acids are classified based on the length and saturation characteristics of the carbon chain. Fatty acids are termed short chain, medium chain, or long chain fatty acids based on the number of carbons present in the chain, are termed saturated fatty acids when no double bonds are present between the carbon atoms, and are termed unsaturated fatty acids when double bonds are present. Unsaturated long chain fatty acids are monounsaturated when only one double bond is present and are polyunsaturated when more than one double bond is present.

Polyunsaturated fatty acids (PUFAs) are classified based on the position of the first double bond from the methyl end of the fatty acid: omega-3 (n-3) fatty acids contain a first double bond at the third carbon, while omega-6 (n-6) fatty acids contain a first double bond at the sixth carbon. For example, docosahexaenoic acid (“DHA”) is an omega-3 long chain polyunsaturated fatty acid (LC-PUFA) with a chain length of 22 carbons and 6 double bonds, often designated as “22:6 n-3.” Other omega-3 LC-PUFAs include eicosapentaenoic acid (“EPA”), designated as “20:5 n-3,” and omega-3 docosapentaenoic acid (“DPA n-3”), designated as “22:5 n-3.” DHA and EPA have been termed “essential” fatty acids. Omega-6 LC-PUFAs include arachidonic acid (“ARA”), designated as “20:4 n-6,” and omega-6 docosapentaenoic acid (“DPA n-6”), designated as “22:5 n-6.”

Omega-3 fatty acids are biologically important molecules that affect cellular physiology due to their presence in cell membranes, regulate production and gene expression of biologically active compounds, and serve as biosynthetic substrates. Roche, H. M., Proc. Nutr. Soc. 58: 397-401 (1999). DHA, for example, accounts for approximately 15%-20% of lipids in the human cerebral cortex and 30%-60% of lipids in the retina, is concentrated in the testes and sperm, and is an important component of breast milk. Jean-Pascal Bergé & Gilles Barnathan, Fatty Acids from Lipids of Marine Organisms: Molecular Biodiversity, Roles as Biomarkers, Biologically Active Compounds, and Economical Aspects, in Marine Biotechnology 149 (T. Scheper, ed., 2005). DHA accounts for up to 97% of the omega-3 fatty acids in the brain and up to 93% of the omega-3 fatty acids in the retina. Moreover, DHA is essential for both fetal and infant development as well as maintenance of cognitive functions in adults. Id. Omega-3 fatty acids, including DHA and EPA, also possess anti-inflammatory properties. See, e.g., id. and Simopoulos, A. P., J. Am. Coll. Nutr. 21: 495-595 (2002). In particular, EPA competes with arachidonic acid for synthesis of eicosanoids such as prostaglandins and leukotrienes through cyclooxygenases and lipoxygenases. Id. Because omega-3 fatty acids are not synthesized de novo in the human body, these fatty acids must be derived from nutritional sources.

Thraustochytrids, which are microalgal organisms of the order Thraustochytriales, are recognized as alternative sources for the production of lipids. For example, strains of thraustrochytrid species have been reported to produce omega-3 fatty acids as a high percentage of the total fatty acids produced by the organisms. See, e.g., U.S. Pat. No. 5,130,242; Huang, J. et al., J. Am. Oil. Chem. Soc. 78: 605-610 (2001); Huang, J. et al., Mar. Biotechnol. 5: 450-457 (2003), incorporated by reference herein in their entireties. Thraustochytrids have also been recognized as sources of lipids for the production of biofuels. See, e.g., U.S. Publ. No. 2009/0064567 and WO 2008/067605, incorporated by reference herein in their entireties.

Plant biomass is composed of three major fractions; cellulose, hemicelluloses, and lignin. Cellulose is composed primarily of glucose polymers, hemicellulose which is enriched with xylose polymers, and lignin which is composed primarily of complex polyphenolic compounds. The carbohydrates derived from these biomass fractions have been referred to collectively as lignocellulosic sugars and have been investigated as a source of low cost renewable feedstocks for fermentation of ethanol.

Plant hemicellulose from sources such as sugar cane bagasse, corn stover, switch grass, wheat straw, hard and soft wood, and the like can be a source of xylose enriched sugars. The sugars can be released from hemicellulose through acid hydrolysis and/or enzymatic digestion.

While thraustochytrids metabolize glucose and/or fructose, they do not appear to naturally metabolize xylose. See, e.g., Honda et al., Mycol. Res. 4:439-448 (1998); Goldstein, American J. of Botany 50:271-279 (1963); Damare, Indian Journal of Marine Sciences 35:326-340 (2006). As such, thraustochytrid cultures, including commercial and industrial cultures, currently require glucose syrups as carbon and energy sources.

A continuing need exists for producing thraustochytrids with the ability to grow on alternative carbon sources such as xylose for use in commercial and industrial applications.

### BRIEF SUMMARY OF THE INVENTION

The present invention is directed to a method of producing a thraustochytrid cell culture, comprising: (a) transforming a thraustochytrid cell with a nucleic acid molecule comprising a polynucleotide sequence encoding a polypeptide associated with xylose import, conversion of xylose to xylulose, phosphorylation of xylulose, or a combination thereof, and (b) growing the transformed thraustochytrid cell in a culture medium comprising xylose as a carbon source. In some embodiments, the polypeptide associated with xylose import, conversion of xylose to xylulose, or phosphorylation of xylulose is selected from the group consisting of: a heterologous xylose transporter, a heterologous xylose isomerase, a heterologous xylulose kinase, a heterologous xylose reductase, a heterologous xylitol dehydrogenase, and combinations thereof. In some embodiments, the thraustochytrid cell expresses a heterologous xylose transporter, a heterologous xylose isomerase, and a heterologous xylulose kinase. In some embodiments, the thraustochytrid cell expresses a heterologous xylose transporter, a heterologous xylose reductase, a heterologous xylitol dehydrogenase, and a heterologous xylulose kinase. In some embodiments, the polynucleotide sequence encoding the polypeptide associated with xylose import is at least 90% identical to a sequence selected from the group consisting of: the polynucleotide sequence of Accession No. AJ875406, BT015128, AF127802, AJ249910, X59465, or X55392; a polynucleotide sequence encoding the amino acid sequence of Accession No. CAB76571; the polynucleotide sequence of SEQ ID NO:2, SEQ ID NO:3, SEQ ID NO:6, SEQ ID NO:7, SEQ ID NO:21, SEQ ID NO:22, or SEQ ID NO:23; and combinations thereof. In some embodiments, the thraustochytrid is a Schizochytrium or a Thraustochytrium. In some embodiments, the invention is directed to a thraustochytrid cell culture produced by the method.

The present invention is also directed to a thraustochytrid cell comprising a nucleic acid molecule comprising a polynucleotide sequence encoding a polypeptide associated with xylose import, conversion of xylose to xylulose, phosphorylation of xylulose, and combinations thereof. In some embodiments, the polypeptide associated with xylose import, conversion of xylose to xylulose, or phosphorylation of xylulose is selected from the group consisting of: a heterologous xylose transporter, a heterologous xylose isomerase, a heterologous xylulose kinase, a heterologous xylose reductase, a heterologous xylitol dehydrogenase, and a combination thereof. In some embodiments, the thraustochytrid cell expresses a heterologous xylose transporter, a heterologous xylose isomerase, and a heterologous xylulose kinase. In some embodiments, the thraustochytrid cell expresses a heterologous xylose transporter, a heterologous xylulose kinase, a heterologous xylose reductase, and a heterologous xylitol dehydrogenase. In some embodiments, the polynucleotide sequence encoding the polypeptide associated with xylose import is at least 90% identical to a sequence selected from the group consisting of: the polynucleotide sequence of Accession No. AJ875406, BT015128, AF127802, AJ249910, X59465, or X55392; a polynucleotide sequence encoding the amino acid sequence of Accession No. CAB76571; the polynucleotide sequence of SEQ ID NO:2, SEQ ID NO:3, SEQ ID NO:6, SEQ ID NO:7, SEQ ID NO:21, SEQ ID NO:22, or SEQ ID NO:23; and combinations thereof. In some embodiments, the thraustochytrid is a Schizochytrium or a Thraustochytrium.

The present invention is also directed to a thraustochytrid culture comprising: (a) any of the thraustochytrid cells described herein, and (b) a cell culture medium comprising xylose as a carbon source.

The present invention is also directed to a method of producing a thraustochytrid biomass, comprising: (a) growing any of the thraustochytrid cells described herein in a culture medium comprising xylose as a carbon source, and (b) harvesting the biomass from the culture medium.

The present invention is also directed to a method of producing a microbial oil, comprising: (a) growing any of the thraustochytrid cells described herein in a culture medium comprising xylose as a carbon source to produce a biomass, and (b) extracting an oil from the biomass.

The present invention is also directed to a method of producing a food product, cosmetic, industrial composition, or pharmaceutical composition for an animal or human, comprising: (a) growing any of the thraustochytrid cells described herein in a culture medium comprising xylose as a carbon source, (b) harvesting a biomass from the culture medium, and (c) preparing the food product, cosmetic, industrial composition, or pharmaceutical composition from the biomass. In some embodiments, the method further comprises extracting an oil from the biomass and preparing the food product, cosmetic, industrial composition, or pharmaceutical composition from the oil. In some embodiments, the food product is an infant formula. In some embodiments, the infant formula is suitable for premature infants. In some embodiments, the food product is milk, a beverage, a therapeutic drink, a nutritional drink, or a combination thereof. In some embodiments, the food product is an additive for animal or human food. In some embodiments, the food product is a nutritional supplement. In some embodiments, the food product is an animal feed. In some embodiments, the animal feed is an aquaculture feed. In some embodiments, the animal feed is a domestic animal feed, a zoological animal feed, a work animal feed, a livestock feed, or a combination thereof.

The present invention is also directed to a method for producing a biofuel, comprising: (a) growing any of the thraustochytrid cells described herein in a culture medium comprising xylose as a carbon source to produce a biomass, (b) extracting an oil from the biomass, and (c) producing a biofuel by transesterifying the oil, cracking the oil, processing the oil by thermal depolymerization, adding the oil to a petroleum refining process, or a combination thereof. In some embodiments, the biofuel is produced by transesterifying the oil. In some embodiments, the biofuel is a biodiesel. In some embodiments, the biofuel is produced by cracking the oil. In some embodiments, the biofuel is a jet biofuel. In some embodiments, the biofuel is produced by processing the oil by thermal depolymerization. In some embodiments, the biofuel is a renewable diesel. In some embodiments, the biofuel is produced by adding the oil to a petroleum refining process. In some embodiments, the biofuel is a co-processed renewable diesel.

### BRIEF DESCRIPTION OF THE DRAWINGS/FIGURES

FIG. 1 shows a plasmid map of pCL0120 (SEQ ID NO:12).

FIG. 2 shows a codon usage table for Schizochytrium.

FIG. 3 shows the codon-optimized polynucleotide sequence (SEQ ID NO:2) encoding the Candida intermedia xylose transporter protein GXS1, corresponding to GenBank Accession No. AJ875406.

FIG. 4 shows the codon-optimized polynucleotide sequence (SEQ ID NO:3) encoding the Arabidopsis thaliana xylose transporter protein At5g17010, corresponding to GenBank Accession No. BT015128.

FIG. 5 shows a plasmid map of pCL0130 (SEQ ID NO:14).

FIG. 6 shows a plasmid map of pCL0131 (SEQ ID NO:15).

FIGS. 7A and 7B shows the polynucleotide sequence of pCL0121 (SEQ ID NO:4). The vector confers resistance to the antibiotic ZEOCIN™ and also harbors a gene encoding a secreted form of eGFP.

FIGS. 8A and 8B shows the polynucleotide sequence of pCL0122 (SEQ ID NO:5). The vector confers resistance to the antibiotic paromomycin and also harbors a gene encoding a secreted form of eGFP.

FIG. 9 shows a plasmid map of pCL0121.

FIG. 10 shows a plasmid map of pCL0122.

FIG. 11 shows the codon-optimized polynucleotide sequence (SEQ ID NO:6) encoding the Piromyces sp. E2 xylose isomerase protein “XylA”, corresponding to GenBank Accession No. CAB76571.

FIG. 12 shows the codon-optimized polynucleotide sequence (SEQ ID NO:7) encoding the Piromyces sp. E2 xylulose kinase protein “XylB”, corresponding to GenBank Accession No. AJ249910.

FIG. 13 shows a plasmid map of pCL0132 (SEQ ID NO:16).

FIG. 14 shows a plasmid map of pCL0136 (SEQ ID NO:20).

FIG. 15 shows the codon-optimized polynucleotide sequence (SEQ ID NO:21) encoding the Pichia stipitis xylose reductase protein “Xyl1” corresponding to GenBank Accession No. X59465.

FIG. 16 shows the codon-optimized polynucleotide sequence (SEQ ID NO:22) encoding the Pichia stipitis xylulose kinase protein “Xuk3” corresponding to GenBank Accession No. AF127802.

FIG. 17 shows a plasmid map of pCL0133 (SEQ ID NO:17).

FIG. 18 shows a plasmid map of pCL0135 (SEQ ID NO:19).

FIG. 19 shows a plasmid map of pCL0123 (SEQ ID NO:13).

FIG. 20 shows the codon-optimized polynucleotide sequence (SEQ ID NO:23) encoding the Pichia stipitis xylitol dehydrogenase protein “Xyl2” corresponding to GenBank Accession No. X55392.

FIG. 21 shows a plasmid map of pCL0134 (SEQ ID NO:18).

FIG. 22 shows Western blots of culture pellet lysates from cultures of SMM-resistant Schizochytrium clones transformed with either pCL0130 (FIG. 22A) or pCL0131 (FIG. 22B) and Schizochytrium wild type strain ATCC 20888 (WT), probed with antibodies that recognize either the Candida intermedia xylose transporter protein GXS1 (pCL0130 transformant) or the Arabidopsis thaliana xylose transporter protein At5g17010 (pCL0131 transformant).

### DETAILED DESCRIPTION OF THE INVENTION

The present invention is directed to recombinant thraustochytrids that grow on xylose and cell cultures comprising the recombinant thraustochytrids, as well as methods of producing cell cultures, biomasses, microbial oils, compositions, and biofuels using the recombinant thraustochytrids.

### Thraustochytrids

According to the present invention, the term “thraustochytrid” refers to any member of the order Thraustochytriales, which includes the family Thraustochytriaceae. The current taxonomic placement of the thraustochytrids can be summarized as follows:

```
Realm: Stramenopila (Chromista)
    Phylum: Labyrinthulomycota (Heterokonta)
        Class: Labyrinthulomycetes (Labyrinthulae)
            Order: Labyrinthulales
                Family: Labyrinthulaceae
            Order: Thraustochytriales
                Family: Thraustochytriaceae
```

For purposes of the present invention, strains described as thraustochytrids include the following organisms: Order: Thraustochytriales; Family: Thraustochytriaceae; Genera: Thraustochytrium (Species: sp., arudimentale, aureum, benthicola, globosum, kinnei, motivum, multirudimentale, pachydermum, proliferum, roseum, striatum), Ulkenia (Species: sp., amoeboidea, kerguelensis, minuta, profunda, radiata, sailens, sarkariana, schizochytrops, visurgensis, yorkensis), Schizochytrium (Species: sp., aggregatum, limnaceum, mangrovei, minutum, octosporum), Japonochytrium (Species: sp., marinum), Aplanochytrium (Species: sp., haliotidis, kerguelensis, profunda, stocchinoi), Althornia (Species: sp., crouchii), or Elina (Species: sp., marisalba, sinorifica). For the purposes of this invention, species described within Ulkenia will be considered to be members of the genus Thraustochytrium. Aurantiochytrium, Oblongichytrium, Botryochytrium, Parietichytrium, and Sicyoidochytrium are additional genuses encompassed by the present invention.

Strains of Thraustochytriales include, but are not limited to, deposited strains PTA-10212, PTA-10213, PTA-10214, PTA-10215, PTA-9695, PTA-9696, PTA-9697, PTA-9698, PTA-10208, PTA-10209, PTA-10210, PTA-10211, Thraustochytrium sp. (23B) (ATCC 20891); Thraustochytrium striatum (Schneider) (ATCC 24473); Thraustochytrium aureum (Goldstein) (ATCC 34304); Thraustochytrium roseum (Goldstein) (ATCC 28210); and Japonochytrium sp. (L1) (ATCC 28207). Schizochytrium include, but are not limited to Schizochytrium aggregatum, Schizochytrium limacinum, Schizochytrium sp. (S31) (ATCC 20888), Schizochytrium sp. (S8) (ATCC 20889), Schizochytrium sp. (LC-RM) (ATCC 18915), Schizochytrium sp. (SR 21), deposited strain ATCC 28209 and deposited Schizochytrium limacinum strain IFO 32693.

In some embodiments, the thraustochytrid cell of the invention is a Schizochytrium or a Thraustochytrium cell. In some embodiments, the thraustochytrid is from a species selected from Schizochytrium sp., Schizochytrium aggregatum, Schizochytrium limacinum, Schizochytrium minutum, Thraustochytrium sp., Thraustochytrium striatum, Thraustochytrium aureum, Thraustochytrium roseum, Japonochytrium sp., and strains derived therefrom.

According to the present invention, the term “transformation” is used to refer to any method by which a nucleic acid molecule (i.e., a recombinant nucleic acid molecule) can be inserted into a thraustochytrid cell of the invention. In microbial systems, the term “transformation” is used to describe an inherited change due to the acquisition of exogenous nucleic acids by the microorganism and is essentially synonymous with the term “transfection.” Suitable transformation techniques for introducing nucleic acid molecules into thraustochytrid cells include, but are not limited to, particle bombardment, electroporation, microinjection, lipofection, adsorption, infection, and protoplast fusion.

Although the phrase “nucleic acid molecule” primarily refers to the physical nucleic acid molecule and the phrases “nucleic acid sequence” or “polynucleotide sequence” primarily refers to the sequence of nucleotides in the nucleic acid molecule, the phrases are used interchangeably, especially with respect to a nucleic acid molecule, polynucleotide sequence, or a nucleic acid sequence that is capable of encoding a protein. In some embodiments, isolated nucleic acid molecules as described herein are produced using recombinant DNA technology (e.g., polymerase chain reaction (PCR) amplification or cloning) or chemical synthesis. In accordance with the present invention, an “isolated” nucleic acid molecule is a nucleic acid molecule that has been removed from its natural milieu (i.e., that has been subject to human manipulation), its natural milieu being the genome or chromosome in which the nucleic acid molecule is found in nature. As such, “isolated” does not necessarily reflect the extent to which the nucleic acid molecule has been purified, but indicates that the molecule does not include an entire genome or an entire chromosome in which the nucleic acid molecule is found in nature. An isolated nucleic acid molecule can include DNA, RNA (e.g., mRNA), or derivatives of either DNA or RNA (e.g., cDNA). Isolated nucleic acid molecules include natural nucleic acid molecules and homologues thereof, including, but not limited to, natural allelic variants and modified nucleic acid molecules in which nucleotides have been inserted, deleted, substituted, and/or inverted in such a manner that such modifications provide the desired effect on sequence, function, and/or the biological activity of the encoded peptide or protein.

The term “protein” includes single-chain polypeptide molecules as well as multiple-polypeptide complexes where individual constituent polypeptides are linked by covalent or non-covalent means. The term “polypeptide” includes peptides of two or more amino acids in length, typically having more than 5, 10, or 20 amino acids. In some embodiments, a polypeptide as described herein is a heterologous polypeptide. The term “heterologous” as used herein refers to a sequence, for example, that is not naturally found in the thraustochytrid cell of the invention.

The present invention is directed to a thraustochytrid cell transformed with a nucleic acid molecule comprising a polynucleotide sequence encoding a polypeptide associated with xylose metabolism. Depending on the native genes present in a thraustochytrid, one or more genes that complete or complement the xylose metabolic pathway can be introduced into a thraustochytrid to allow for growth on xylose. In certain embodiments, the polypeptide or a combination of polypeptides is involved in the conversion of xylose to xylulose-5-phosphate (e.g., for entry to the pentose phosphate pathway). In certain embodiments, the conversion of xylose to xylulose-5-phosphate includes 3 steps: xylose import, conversion of xylose to xylulose, and phosphorylation of xylulose. In certain embodiments, a polypeptide of the invention is involved in xylose import (e.g., a xylose symporter or a xylose transporter), conversion of xylose to xylulose either directly (e.g., a xylose isomerase) or via the intermediate sugar alcohol xylitol (e.g., a xylose reductase or a xylitol dehydrogenase), and/or phosphorylation of xylulose (e.g., a xylulose kinase). In certain embodiments, a thraustochytrid cell is transformed with one or more polynucleotide sequences encoding a polypeptide directed to xylose import, conversion of xylose to xylulose, and/or phosphorylation of xylulose.

In some embodiments, the polypeptide is a heterologous polypeptide. In some embodiments, a thraustochytrid cell of the invention comprises a polynucleotide sequence encoding a heterologous xylose transporter (e.g., accession nos. AJ875406 and/or BT015128), heterologous xylose isomerase (e.g., accession no. CAB76571), heterologous xylulose kinase (e.g., accession no. AF127802 and/or AJ249910), heterologous xylose reductase (e.g., accession no. X59465), heterologous xylitol dehydrogenase (e.g., accession no. X55392), or any combination thereof. In some embodiments, the thraustochytrid cell comprises a polynucleotide sequence encoding a xylose transporter (e.g., accession nos. AJ875406 and/or BT015128), a xylose isomerase (e.g., accession no. CAB76571), and a xylulose kinase (e.g., accession nos. AF127802 and/or AJ249910). In some embodiments, the xylose isomerase is as described in WO 03/062430 A1. In some embodiments, the polynucleotide sequence is codon-optimized to maximize translation efficiency in the thraustochytrid cell. In some embodiments, a polynucleotide sequence encoding a xylose transporter, a xylose isomerase, a xylulose kinase, a xylose reductase, a xylitol dehydrogenase, or any combination thereof is integrated into the genome of the thraustochytrid cell. In some embodiments, a polynucleotide sequence encoding a xylose transporter, a xylose isomerase, a xylulose kinase, a xylose reductase, a xylitol dehydrogenase, or any combination thereof is stably integrated into the genome of the thraustochytrid cell.

In some embodiments, a thraustochytrid cell of the invention comprises one or more nucleic acid molecules comprising one or more polynucleotide sequences encoding three or more polypeptides associated with xylose import, conversion of xylose to xylulose, or phosphorylation of xylulose. In some embodiments, a combination of three or more polypeptides can include one or more polypeptides associated with xylose import, one or more polypeptides associated with conversion of xylose to xylulose, and one or more polypeptides associated with phosphorylation of xylulose. In some embodiments, a combination of three or more polypeptides can include two or more polypeptides associated with xylose import, one or more polypeptides associated with conversion of xylose to xylulose, and zero or more polypeptides associated with phosphorylation of xylulose. In some embodiments, a combination of three or more polypeptides can include two or more polypeptides associated with xylose import, zero or more polypeptides associated with conversion of xylose to xylulose, and one or more polypeptides associated with phosphorylation of xylulose. In some embodiments, a combination of three or more polypeptides can include one or more polypeptides associated with xylose import, two or more polypeptides associated with conversion of xylose to xylulose, and zero or more polypeptides associated with phosphorylation of xylulose. In some embodiments, a combination of three or more polypeptides can include one or more polypeptides associated with xylose import, zero or more polypeptides associated with conversion of xylose to xylulose, and two or more polypeptides associated with phosphorylation of xylulose. In some embodiments, a combination of three or more polypeptides can include zero or more polypeptides associated with xylose import, two or more polypeptides associated with conversion of xylose to xylulose, and one or more polypeptides associated with phosphorylation of xylulose. In some embodiments, a combination of three or more polypeptides can include zero or more polypeptides associated with xylose import, one or more polypeptides associated with conversion of xylose to xylulose, and two or more polypeptides associated with phosphorylation of xylulose. In some embodiments, a combination of three or more polypeptides can include a heterologous xylose transporter, a heterologous xylulose kinase, and a heterologous xylose isomerase. In some embodiments, a combination of three or more polypeptides can include a heterologous xylose transporter, a heterologous xylulose kinase, and a heterologous xylose reductase. In some embodiments, a combination of three or more polypeptides can include a heterologous xylose transporter, a heterologous xylulose kinase, and a heterologous xylitol dehydrogenase. In some embodiments, a combination of three or more polypeptides can include a heterologous xylose transporter, a heterologous xylulose kinase, a heterologous xylose reductase, and a heterologous xylitol dehydrogenase. In some embodiments, a combination of three or more polypeptides can include a heterologous xylose transporter, a heterologous xylulose kinase, a heterologous xylose isomerase, and a heterologous xylitol dehydrogenase. In some embodiments, a combination of three or more polypeptides can include a heterologous xylose transporter, a heterologous xylulose kinase, a heterologous xylose reductase, and a heterologous xylose isomerase. In some embodiments, any of the foregoing combinations include combinations in which any of the polypeptides associated with xylose import, conversion of xylose to xylulose, and phosphorylation of xylulose are from different organisms.

In some embodiments, the expression system used for the production of a xylose transporter, a xylose isomerase, a xylulose kinase, a xylose reductase, a xylitol dehydrogenase, or any combination thereof in a thraustochytrid cell comprises regulatory elements that are derived from thraustochytrid sequences. In some embodiments, the expression system used for the production of a xylose transporter, a xylose isomerase, a xylulose kinase, a xylose reductase, a xylitol dehydrogenase, or any combination thereof in a thraustochytrid cell comprises regulatory elements that are derived from non-thraustochytrid sequences, including sequences derived from non-thraustochytrid algal sequences. In some embodiments, the expression system comprises a polynucleotide sequence encoding a xylose transporter, a xylose isomerase, a xylulose kinase, a xylose reductase, a xylitol dehydrogenase, or any combination thereof wherein the polynucleotide sequence is operably linked to any promoter sequence, any terminator sequence, and/or any other regulatory sequence that is functional in a thraustochytrid cell. Inducible or constitutively active regulatory sequences can be used. Regulatory sequences include but are not limited to the Schizochytrium regulatory sequences described in U.S. Publ. No. 2010/0233760, U.S. Pat. No. 7,001,772, U.S. Publ. No. 2006/0275904, and U.S. Publ. No. 2006/0286650, incorporated by reference herein in their entireties, such as: an OrfC (also termed Pfa3) promoter, an OrfC terminator, an EF1 short promoter, an EF1 long promoter, a Sec1 promoter, a 60S short promoter, a 60S long promoter, an acetolactate synthase promoter, an acetolactate synthase terminator, an α-tubulin promoter, a promoter from a polyketide synthase (PKS) system, a fatty acid desaturase promoter, an actin promoter, an actin terminator, an elongation factor 1 alpha (ef1α) promoter, an ef1α terminator, a glyceraldehyde 3-phosphate dehydrogenase (gapdh) promoter, a gapdh terminator, and combinations thereof.

In some embodiments, the xylose transporter, xylose isomerase, xylulose kinase, xylose reductase, xylitol dehydrogenase, or any combination thereof is expressed by a recombinant thraustochytrid transformed with a xylose transporter gene, a xylose isomerase gene, a xylulose kinase gene, a xylose reductase gene, a xylitol dehydrogenase gene, or any combination thereof. In some embodiments, any of one or more of the xylose transporters is a plasma-membrane bound protein. In some embodiments, any of one or more of the xylose isomerase, xylulose kinase, xylose reductase, or xylitol dehydrogenase is an intracellular protein.

In some embodiments, the xylose transporter, xylose isomerase, xylulose kinase, a xylose reductase, xylitol dehydrogenase, or any combination thereof is expressed intracellularly. In some embodiments, the xylose transporters are membrane bound. In certain embodiments, the xylose transporters are localized to the exterior of the plasma membrane.

In some embodiments, an expression cassette is used for the expression of a xylose transporter, a xylose isomerase, a xylulose kinase, a xylose reductase, a xylitol dehydrogenase, or any combination thereof in a thraustochytrid cell. The design and construction of expression cassettes use standard molecular biology techniques known to persons skilled in the art. See, e.g., Sambrook et al., 2001, Molecular Cloning: A Laboratory Manual, 3^{rd} edition. In some embodiments, the thraustochytrid cell comprises an expression cassette containing genetic elements, such as at least the following, operably linked in such a way that they are functional in the thraustochytrid cell: a promoter; a coding sequence comprising a xylose transporter, a xylose isomerase, a xylulose kinase, a xylose reductase, a xylitol dehydrogenase, or any combination thereof; and a terminator region. In some embodiments, the expression cassette further comprises a polynucleotide sequence encoding a signal peptide or a membrane domain operably linked to the xylose transporter, xylose isomerase, xylulose kinase, xylose reductase, or xylitol dehydrogenase. The term “membrane domain” as used herein refers to any domain within a polypeptide that targets the polypeptide to a membrane and/or allows the polypeptide to maintain association with a membrane and includes, but is not limited to, a transmembrane domain (e.g., a single or multiple membrane spanning region), an integral monotopic domain, a signal anchor sequence, an ER signal sequence, an N-terminal or internal or C-terminal stop transfer signal, a glycosylphosophatidylinositol anchor, and combinations thereof. A membrane domain can be located at any position in the polypeptide, including the N-terminal, C-terminal, or middle of the polypeptide. A membrane domain can be associated with permanent or temporary attachment of a polypeptide to a membrane. In some embodiments, a recombinant vector comprises the expression cassette. Recombinant vectors include, but are not limited to, plasmids, phages, and viruses. In some embodiments, the recombinant vector is a linearized vector. In some embodiments, the recombinant vector is an expression vector. As used herein, the phrase “expression vector” refers to a vector that is suitable for production of an encoded product (e.g., a xylose transporter, a xylose isomerase, a xylulose kinase, a xylose reductase, a xylitol dehydrogenase, or any combination thereof). In some embodiments, a polynucleotide sequence encoding the xylose transporter, xylose isomerase, xylulose kinase, xylose reductase, xylitol dehydrogenase, or any combination thereof is inserted into the recombinant vector to produce a recombinant nucleic acid molecule. In some embodiments, the polynucleotide sequence encoding the xylose transporter, xylose isomerase, xylulose kinase, xylose reductase, xylitol dehydrogenase, or any combination thereof is inserted into the vector in a manner that operatively links the nucleic acid sequence to regulatory sequences in the vector, which enables the transcription and translation of the nucleic acid sequence within the recombinant microorganism. In some embodiments, a selectable marker enables the selection of a recombinant microorganism into which a recombinant nucleic acid molecule of the present invention has successfully been introduced. Selectable markers include but are not limited to the selectable markers described in U.S. Publ. No. 2010/0233760 and U.S. Pat. No. 7,001,772, incorporated by reference herein in their entireties, such as Schizochytrium acetolactate synthase or bacterial ble. In some embodiments, the selection marker is an auxotrophic marker, a dominant selection marker (such as, for example, an enzyme that detoxifies an antibiotic), or another protein involved in transformation selection.

In some embodiments, a polynucleotide sequence of any of the methods of the invention encodes a heterologous polypeptide comprising an amino acid sequence that is at least 90%, at least 91%, at least 92%, at least 93%, at least 94%, at least 95%, at least 96%, at least 97%, at least 98%, at least 99%, or that is identical to the amino acid sequence of a polypeptide associated with xylose metabolism including, but not limited to, a polypeptide associated with xylose import, conversion of xylose to xylulose, or phosphorylation of xylulose. In some embodiments, a polynucleotide sequence of any of the methods of the invention encodes a heterologous polypeptide comprising an amino acid sequence that is at least 90%, at least 91%, at least 92%, at least 93%, at least 94%, at least 95%, at least 96%, at least 97%, at least 98%, at least 99%, or that is identical to the amino acid sequence of a xylose symporter, xylose transporter, xylose isomerase, xylose reductase, xylitol dehydrogenase, or xylulose kinase. In some embodiments, a polynucleotide sequence of any of the methods of the invention encodes a polypeptide comprising an amino acid sequence that is at least 90%, at least 91%, at least 92%, at least 93%, at least 94%, at least 95%, at least 96%, at least 97%, at least 98%, at least 99%, or that is identical to: the amino acid sequence encoded by Accession No. AJ875406, BT015128, AF127802, AJ249910, X59465, or X55392; or the amino acid sequence of Accession No. CAB76571. In some embodiments, a polynucleotide sequence of any of the methods of the invention encodes a polypeptide comprising an amino acid sequence that is at least 90%, at least 91%, at least 92%, at least 93%, at least 94%, at least 95%, at least 96%, at least 97%, at least 98%, at least 99%, or that is identical to the amino acid sequence encoded by SEQ ID NO:2, SEQ ID NO:3, SEQ ID NO:6, SEQ ID NO:7, SEQ ID NO:21, SEQ ID NO:22, or SEQ ID NO:23. In some embodiments, a polynucleotide sequence of any of the methods of the invention is at least 90%, at least 91%, at least 92%, at least 93%, at least 94%, at least 95%, at least 96%, at least 97%, at least 98%, at least 99%, or that is identical to: the polynucleotide sequence of Accession No. AJ875406, BT015128, AF127802, AJ249910, X59465, or X55392; a polynucleotide sequence of Accession No. AJ875406, BT015128, AF127802, AJ249910, X59465, or X55392 that is codon-optimized for expression in Schizochytrium; a polynucleotide sequence encoding the amino acid sequence of Accession No. CAB76571; a polynucleotide sequence encoding the amino acid sequence of accession no. CAB76571, wherein the polynucleotide sequence is codon-optimized for expression in Schizochytrium; or the polynucleotide sequence of SEQ ID NO:2, SEQ ID NO:3, SEQ ID NO:6, SEQ ID NO:7, SEQ ID NO:21, SEQ ID NO:22, or SEQ ID NO:23.

### Thraustochytrid Cultures, Biomasses, and Microbial Oils

The present invention is also directed to a thraustochytrid cell culture comprising any of the thraustochytrid cells of the invention as described herein and a cell culture medium comprising xylose as a carbon source.

The present invention is also directed to a method of producing a thraustochytrid cell culture, comprising transforming a thraustochytrid cell with a nucleic acid molecule comprising a polynucleotide sequence encoding a polypeptide associated with xylose metabolism as described herein, selecting the transformed thraustochytrid cell, and growing the transformed thraustochytrid cell in a culture medium comprising xylose as a carbon source, wherein a thraustochytrid cell culture is produced. The present invention is also directed to a thraustochytrid cell culture produced by the method. Sources of xylose include but are not limited to sugar cane bagasse, corn stover, switch grass, wheat straw, hard and softwood, or other plant material.

In some embodiments, xylose is the primary carbon source in the cell culture medium. In some embodiments, xylose is the only carbon source in the cell culture medium. In some embodiments, the culture medium comprises molasses, a syrup, hydrolysate, extract, or juice from any xylose-producing plant, or combinations thereof. In some embodiments, the culture medium comprises a hemicellulose-containing feedstock, such as sugar cane bagasse, corn stover, switch grass, wheat straw, hard and soft wood or other plant material.

In some embodiments, the cell culture medium comprises a xylose transporter, a xylose isomerase, a xylulose kinase, a xylose reductase, a xylitol dehydrogenase, or any combination thereof. In some embodiments, the thraustochytrid cell exports a heterologous xylose transporter, a heterologous xylose isomerase, a heterologous xylulose kinase, a heterologous xylose reductase, a heterologous xylitol dehydrogenase, or any combination thereof into the culture medium.

Culture conditions for thraustochytrid cells include, but are not limited to, effective media, bioreactor, temperature, pH, and oxygen conditions that permit protein production and/or recombination. An effective medium refers to any medium in which a thraustochytrid cell is typically cultured. Such medium typically comprises an aqueous medium having assimilable carbon, nitrogen, and phosphate sources, as well as appropriate salts, minerals, metals, and other nutrients, such as vitamins. Nitrogen sources include, but are not limited to, peptone, yeast extract, polypeptone, malt extract, meat extract, casamino acid, corn steep liquor, organic nitrogen sources, sodium glutamate, urea, inorganic nitrogen sources, ammonium acetate, ammonium sulfate, ammonium chloride, ammonium nitrate, and sodium sulfate. Non-limiting culture conditions suitable for thraustochytrids are described, for example, in U.S. Pat. No. 5,340,742. Various fermentation parameters for inoculating, growing, and recovering microflora are also described, for example, in U.S. Pat. No. 5,130,242. Liquid or solid media can contain natural or artificial sea water. Thraustochytrid cells of the present invention can be cultured in fermentation bioreactors, shake flasks, test tubes, microtiter dishes, and petri plates.

The fermentation volume can be any volume used for the growth of thraustochytrids, including commercial and industrial volumes. In some embodiments, the fermentation volume (volume of culture) is at least 2 L, at least 10 L, at least 50 L, at least 100 L, at least 200 L, at least 500 L, at least 1000 L, at least 10,000 L, at least 20,000 L, at least 50,000 L, at least 100,000 L, at least 150,000 L, at least 200,000 L, or at least 250,000 L, at least 300,000 L, at least 350,000 L, at least 400,000 L, or at least 500,000 L. In some embodiments, the fermentation volume is 2 L to 2,000,000 L, 2 L to 1,000,000 L, 2 L to 500,000 L, 2 L to 200,000 L, 2 L to 100,000 L, 2 L to 50,000 L, 2 L to 25,000 L, 2 L to 20,000 L, 2 L to 15,000 L, 2 L to 10,000 L, 2 L to 5,000 L, 2 L to 1,000 L, 2 L to 500 L, or 2 L to 100 L.

The present invention is also directed to a method of producing a thraustochytrid biomass comprising growing a thraustochytrid cell of the invention in a culture medium comprising xylose as a carbon source, and harvesting a biomass from the culture medium. A thraustochytrid biomass is a harvested cellular biomass obtained by any conventional method for the isolation of a thraustochytrid biomass, such as described in U.S. Pat. No. 5,130,242 and U.S. Publ. No. 2002/0001833, incorporated by reference herein in their entireties. A harvested biomass can contain thraustochytrid cells as well as thraustochytrid cellular fragments.

The present invention is also directed to a method of producing a microbial oil comprising growing a thraustochytrid cell of the invention in a culture medium comprising xylose as a carbon source to produce a biomass, and extracting an oil from the biomass. The oil can be extracted from a freshly harvested biomass or can be extracted from a previously harvested biomass that has been stored under conditions that prevent spoilage. Known methods can be used to culture a thraustochytrid of the invention, to isolate a biomass from the culture, to extract microbial oil from the biomass, and to analyze the fatty acid profile of oils extracted from the biomass. See, e.g., U.S. Pat. No. 5,130,242.

A microbial oil can be any oil derived from any thraustochytrid, including, for example: a crude oil extracted from the biomass of a thraustochytrid without further processing; a refined oil that is obtained by treating a crude oil with further processing such as refining, bleaching, and/or deodorizing; a diluted oil obtained by diluting a crude or refined oil; or an enriched oil that is obtained, for example, by treating a crude or refined oil with further methods of purification to increase the concentration of a fatty acid in the oil.

In some embodiments, the crude oil can be isolated from a thraustochytrid using standard techniques, without being subjected to further refinement or purification. For example, the crude oil can be isolated using solvent extraction, such as, but not limited to, hexane extraction or isopropanol extraction. In some embodiments, the crude oil can be isolated using physical and/or mechanical extraction methods such as, but not limited to, extraction through the use of a homogenizer or by pressing.

In some embodiments, the crude oil can be subjected to further processing, such as refining, desolventization, deodorization, winterization, chill filtration, and/or bleaching. Such “processed” oils include microbial oils that have been subjected to solvent extraction and one or more further processing. In some embodiments, oils are minimally processed. “Minimally processed” oils include microbial oils that have been subjected to solvent extraction and filtration. In certain embodiments, minimally processed oils are further subjected to winterization.

In some embodiments, a method similar to the FRIOLEX® process (Westfalia Separator Industry GmbH, Germany) is used to extract the microbial oils produced by the thraustochytrids. The FRIOLEX® process is a water-based physical oil extraction process, whereby raw material containing oil can be used directly for extracting oil without using any conventional solvent extraction methods. In this process, a water-soluble organic solvent can be used as a process aid and the oil is separated from the raw material broth by density separation using gravity or centrifugal forces. WO 96/05278 discloses such extraction methods and is herein incorporated by reference in its entirety.

After the oil has been extracted, the oil can be recovered or separated from non-lipid components by any suitable means known in the art. In some embodiments, low-cost physical and/or mechanical techniques are used to separate the lipid-containing compositions from non-lipid compositions. For example, if multiple phases or fractions are created by the extraction method used to extract the oil, where one or more phases or fractions contain lipids, a method for recovering the lipid-containing phases or fractions can involve physically removing the lipid-containing phases or fractions from the non-lipid phases or fractions, or vice versa. In some embodiments, a FRIOLEX® type method is used to extract the lipids produced by the microorganisms, and the lipid-rich light phase is then physically separated from the protein-rich heavy phase (such as by skimming off the lipid-rich phase that is on top of the protein-rich heavy phase after density separation).

The microbial oils produced by thraustochytrids of the present invention can be recovered from autolysis or induced lysis by exposing thraustochytrid cells to a condition including, but not limited to, a certain pH, a certain temperature, the presence of an enzyme, the presence of a detergent, physical disruptions, or combinations thereof. In some embodiments, lysis or autolysis of the thraustochytrid cells is performed by the use of mechanical forces. In further embodiments of the present invention, the lysis or autolysis of thraustochytrid cells is followed by mechanical separation of the lipids from the non-lipid compositions.

Suitable enzymes that can be used to induce lysis of the thraustochytrid cells include, but are not limited to, commercially available enzymes or enzyme mixtures such as Proteinase K or ALCALASE®. In some embodiments, the oil-producing thraustochytrids undergo induced lysis in the presence of a detergent such as ionic (cationic or anionic) detergents, nonionic detergents, zwitterionic detergents, or combinations thereof. In further embodiments of the present invention, physical disruption methods such as mechanical grinding, liquid homogenization, use of high frequency sound waves in sonication, freeze/thaw cycles methods, pressing, extruding, or milling can be used to induce lysis of the oil-producing thraustochytrids. The extraction of the oils can take place in the fermentors at the end of the fermentation by in-tank lysis of the thraustochytrid cells.

In some embodiments, the cell cultures, biomasses, and microbial oils produced from the thraustochytrid cells of the invention grown on xylose have the same or substantially similar characteristics (e.g., cell densities, dry cell weights, fatty acid productivities, and fatty acid profiles) associated with cultures, biomasses, and microbial oils produced from the corresponding untransformed thraustochytrid cells grown on glucose and/or fructose. In some embodiments, the dry cell weight of a biomass produced from a thraustochytrid culture of the invention is higher after growth on xylose than the dry cell weight obtained under identical conditions from growth of a culture of the corresponding untransformed thraustochytrid cells on glucose and/or fructose. In some embodiments, the total amount of fatty acids as a percentage of the dry cell weight of the biomass is higher after growth of a thraustochytrid of the invention on xylose than the amount obtained under identical conditions from growth of a corresponding untransformed thraustochytrid cell on glucose and/or fructose.

### Methods of Producing Compositions

The present invention is also directed to a method of preparing a food product, cosmetic, industrial composition, or pharmaceutical composition for animals or humans, comprising growing a thraustochytrid cell of the invention in a culture medium comprising xylose as a carbon source to produce a biomass, harvesting the biomass, and preparing the food product, cosmetic, industrial composition, or pharmaceutical composition for animals or humans from the biomass.

Thraustochytrid biomasses can be dried prior to use in a composition by methods including, but not limited to, freeze drying, air drying, spray drying, tunnel drying, vacuum drying (lyophilization), or a similar process. Alternatively, a harvested and washed biomass can be used directly in a composition without drying. See, e.g., U.S. Pat. Nos. 5,130,242 and 6,812,009, incorporated by reference herein in their entireties.

In some embodiments, the method of preparing a food product, cosmetic, industrial composition, or pharmaceutical composition for animals or humans, further comprises extracting oil from the biomass. Microbial oils can be used as starting material to more efficiently produce a product enriched in a fatty acid such as DHA or EPA. For example, the microbial oils can be subjected to various purification techniques known in the art, such as distillation or urea adduction, to produce a higher potency product with higher concentrations of DHA, EPA, or another fatty acid. The microbial oils can also be used in chemical reactions to produce compounds derived from fatty acids in the oils, such as esters and salts of DHA, EPA, or another fatty acid.

Any thraustochytrid that has been transformed as described herein to grow on xylose can be selected for preparing any of the described compositions based on a desirable attribute of the thraustochytrid, such as but not limited to the cell density of a cell culture of the thraustochytrid, the percentages and types of fatty acids associated with the dry cell of a biomass of the thraustochytrid, the fatty acid profile associated with the biomass and/or the microbial oil of the thraustochytrid, or a combination thereof. In some embodiments, the thraustochytrid biomass or microbial oil comprises a greater percentage of polyunsaturated fatty acids than monounsaturated fatty acids. In some embodiments, the thraustochytrid biomass or microbial oil comprises a greater percentage of omega-3 fatty acids than omega-6 fatty acids. In some embodiments, the thraustochytrid biomass or microbial oil comprises a greater percentage of DHA than other omega-3 fatty acids. In some embodiments, the thraustochytrid biomass comprises a greater percentage of DHA than other polyunsaturated fatty acids. In some embodiments, the thraustochytrid biomass or microbial oil comprises a greater percentage of EPA than other omega-3 fatty acids. In some embodiments, the thraustochytrid biomass or microbial oil comprises a greater percentage of EPA than other polyunsaturated fatty acids.

A composition can include one or more excipients. As used herein, “excipient” refers to a component, or mixture of components, that is used in a composition to give desirable characteristics to the composition, including foods as well as pharmaceutical, cosmetic, and industrial compositions. An excipient can be described as a “pharmaceutically acceptable” excipient when added to a pharmaceutical composition, meaning that the excipient is a compound, material, composition, salt, and/or dosage form which is, within the scope of sound medical judgment, suitable for contact with the tissues of human beings and animals without excessive toxicity, irritation, allergic response, or other problematic complications over the desired duration of contact commensurate with a reasonable benefit/risk ratio. In some embodiments, the term “pharmaceutically acceptable” means approved by a regulatory agency of the Federal or a state government or listed in the U.S. Pharmacopeia or other generally recognized international pharmacopeia for use in animals, and more particularly in humans. Various excipients can be used. In some embodiments, the excipient can be, but is not limited to, an alkaline agent, a stabilizer, an antioxidant, an adhesion agent, a separating agent, a coating agent, an exterior phase component, a controlled-release component, a solvent, a surfactant, a humectant, a buffering agent, a filler, an emollient, or combinations thereof. Excipients in addition to those discussed herein can include excipients listed in, though not limited to, Remington: The Science and Practice of Pharmacy, 21^{st} ed. (2005).

In some embodiments, the composition is a food product. A food product is any food for animal or human consumption, and includes both solid and liquid compositions. A food product can be an additive to animal or human foods. Foods include, but are not limited to, common foods; liquid products, including milks, beverages, therapeutic drinks, and nutritional drinks; functional foods; supplements; nutraceuticals; infant formulas, including formulas for pre-mature infants; foods for pregnant or nursing women; foods for adults; geriatric foods; and animal foods.

In some embodiments, a thraustochytrid biomass or microbial oil can be used directly as or included as an additive within one or more of: an oil, shortening, spread, other fatty ingredient, beverage, sauce, dairy-based or soy-based food (such as milk, yogurt, cheese and ice-cream), a baked good, a nutritional product, e.g., as a nutritional supplement (in capsule or tablet form), a vitamin supplement, a diet supplement, a powdered drink, a finished or semi-finished powdered food product, and combinations thereof.

A partial list of food compositions that can include a microbial oil includes, but is not limited to, soya based products (milks, ice creams, yogurts, drinks, creams, spreads, whiteners); soups and soup mixes; doughs, batters, and baked food items including, for example, fine bakery wares, breakfast cereals, cakes, cheesecakes, pies, cupcakes, cookies, bars, breads, rolls, biscuits, muffins, pastries, scones, croutons, crackers, sweet goods, snack cakes, pies, granola/snack bars, and toaster pastries; candy; hard confectionery; chocolate and other confectionery; chewing gum; liquid food products, for example milks, energy drinks, infant formula, carbonated drinks, teas, liquid meals, fruit juices, fruit-based drinks, vegetable-based drinks; multivitamin syrups, meal replacers, medicinal foods, and syrups; powdered beverage mixes; pasta; processed fish products; processed meat products; processed poultry products; gravies and sauces; condiments (ketchup, mayonnaise, etc.); vegetable oil-based spreads; dairy products; yogurt; butters; frozen dairy products; ice creams; frozen desserts; frozen yogurts; semi-solid food products such as baby food; puddings and gelatin desserts; processed and unprocessed cheese; pancake mixes; food bars including energy bars; waffle mixes; salad dressings; replacement egg mixes; nut and nut-based spreads; salted snacks such as potato chips and other chips or crisps, corn chips, tortilla chips, extruded snacks, popcorn, pretzels, potato crisps, and nuts; specialty snacks such as dips, dried fruit snacks, meat snacks, pork rinds, health food bars, and rice/corn cakes.

In some embodiments, a microbial oil can be used to supplement infant formula. Infant formula can be supplemented with a microbial oil alone or in combination with a physically refined oil derived from an arachidonic acid (ARA)-producing microorganism. An ARA-producing microorganism, for example, is Mortierella alpina or Mortierella sect. schmuckeri. Alternatively, infant formulas can be supplemented with a microbial oil in combination with an oil rich in ARA, including ARASCO® (Martek Biosciences, Columbia, Md.).

In some embodiments, the composition is an animal feed. An “animal” means any non-human organism belonging to the kingdom Animalia, and includes, without limitation, aquatic animals and terrestrial animals. The term “animal feed” or “animal food” refers to any food intended for non-human animals, whether for fish; commercial fish; ornamental fish; fish larvae; bivalves; mollusks; crustaceans; shellfish; shrimp; larval shrimp; artemia; rotifers; brine shrimp; filter feeders; amphibians; reptiles; mammals; domestic animals; farm animals; zoo animals; sport animals; breeding stock; racing animals; show animals; heirloom animals; rare or endangered animals; companion animals; pet animals such as dogs, cats, guinea pigs, rabbits, rats, mice, or horses; primates such as monkeys (e.g., cebus, rhesus, African green, patas, cynomolgus, and cercopithecus), apes, orangutans, baboons, gibbons, and chimpanzees; canids such as dogs and wolves; felids such as cats, lions, and tigers; equids such as horses, donkeys, and zebras; food animals such as cows, cattle, pigs, and sheep; ungulates such as deer and giraffes; rodents such as mice, rats, hamsters and guinea pigs; and so on. An animal feed includes, but is not limited to, an aquaculture feed, a domestic animal feed including pet feed, a zoological animal feed, a work animal feed, a livestock feed, or a combination thereof.

In some embodiments, the composition is a feed or feed supplement for any animal whose meat or products are consumed by humans, such as any animal from which meat, eggs, or milk is derived for human consumption. When fed to such animals, nutrients such as LC-PUFAs can be incorporated into the flesh, milk, eggs or other products of such animals to increase their content of these nutrients.

In some embodiments, the composition is a spray-dried material that can be crumbled to form particles of an appropriate size for consumption by zooplankton, artemia, rotifers, and filter feeders. In some embodiments, the zooplankton, artemia, or rotifers fed by the composition are in turn fed to fish larvae, fish, shellfish, bivalves, or crustaceans.

In some embodiments, the composition is a pharmaceutical composition. Suitable pharmaceutical compositions include, but are not limited to, an anti-inflammatory composition, a drug for treatment of coronary heart disease, a drug for treatment of arteriosclerosis, a chemotherapeutic agent, an active excipient, an osteoporosis drug, an anti-depressant, an anti-convulsant, an anti-Helicobacter pylori drug, a drug for treatment of neurodegenerative disease, a drug for treatment of degenerative liver disease, an antibiotic, a cholesterol lowering composition, and a triglyceride lowering composition. In some embodiments, the composition is a medical food. A medical food includes a food that is in a composition to be consumed or administered externally under the supervision of a physician and that is intended for the specific dietary management of a condition, for which distinctive nutritional requirements, based on recognized scientific principles, are established by medical evaluation.

In some embodiments, the microbial oil can be formulated in a dosage form. Dosage forms can include, but are not limited to, tablets, capsules, cachets, pellets, pills, powders and granules, and parenteral dosage forms, which include, but are not limited to, solutions, suspensions, emulsions, and dry powders comprising an effective amount of the microbial oil. It is also known in the art that such formulations can also contain pharmaceutically acceptable diluents, fillers, disintegrants, binders, lubricants, surfactants, hydrophobic vehicles, water soluble vehicles, emulsifiers, buffers, humectants, moisturizers, solubilizers, preservatives, and the like. Administration forms can include, but are not limited to, tablets, dragees, capsules, caplets, and pills, which contain the microbial oil and one or more suitable pharmaceutically acceptable carriers.

For oral administration, the microbial oil can be combined with pharmaceutically acceptable carriers well known in the art. Such carriers enable the microbial oils to be formulated as tablets, pills, dragees, capsules, liquids, gels, syrups, slurries, suspensions, and the like, for oral ingestion by a subject to be treated. In some embodiments, the dosage form is a tablet, pill, or caplet. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl cellulose, sodium carboxymethyl cellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate. Pharmaceutical preparations that can be used orally include, but are not limited to, push-fit capsules made of gelatin, as well as soft, sealed capsules made of gelatin and a plasticizer, such as glycerol or sorbitol.

In some embodiments, the composition is a cosmetic. Cosmetics include, but are not limited to, emulsions, creams, lotions, masks, soaps, shampoos, washes, facial creams, conditioners, make-ups, bath agents, and dispersion liquids. Cosmetic agents can be medicinal or non-medicinal.

In some embodiments, the composition is an industrial composition. In some embodiments, the composition is a starting material for one or more manufactures. A manufacture includes, but is not limited to, a polymer; a photographic photosensitive material; a detergent; an industrial oil; or an industrial detergent. For example, U.S. Pat. No. 7,259,006 describes use of DHA-containing fat and oil for production of behenic acid and production of photographic sensitive materials using behenic acid.

### Methods of Producing Biofuels

The present invention is also directed to a method for producing a biofuel from a thraustochytrid of the invention.

As used herein, “biofuel” refers to any fuel that is produced from or using a biomass or a microbial oil of a thraustochytrid of the invention, including, but not limited to, a biodiesel, a renewable diesel, a co-processed renewable diesel, a jet biofuel, a heating oil, and a fuel additive. Any method known in the art can be used to generate a biofuel from the biomasses and microbial oils of the thraustochytrids of the present invention, including but not limited to any methods described in WO 2005/035693, U.S. Publ. No. 2005/0112735, WO 2007/027633, WO 2006/127512, U.S. Publ. No. 2007/0099278, U.S. Publ. No. 2007/0089356, WO 2008/067605, U.S. Publ. No. 2009/0035842, and U.S. Publ. No. 2009/0064567, incorporated by reference herein in their entireties. Any of the biofuels described herein can be blended with a fossil fuel. For example, a biodiesel can be blended with a fossil diesel at ratios of 1%-99%. In some embodiments, a microbial oil of a thraustochytrid of the invention is used as a starting point for production of a biofuel even though it has not been subjected to conventional processing. Examples of conventional processing that can be avoided include refining (e.g., physical refining, silica refining or caustic refining), desolventization, deodorization, winterization, chill filtration, and/or bleaching. Thus, in certain embodiments, the oils have not been subjected to refining, desolventization, deodorization, winterization, chill filtration, bleaching, or a combination thereof.

In some embodiments, the method of producing a biofuel comprises growing a thraustochytrid of the invention in a culture medium comprising xylose as a carbon source to produce a biomass, extracting an oil from the biomass, and producing a biofuel by transesterifying the oil, cracking the oil, processing the oil by thermal depolymerization, adding the oil to a traditional petroleum refining process, or a combination thereof.

In some embodiments, the biofuel is produced by transesterifying the oil. In some embodiments, the biofuel is a biodiesel. Various forms of biodiesel are known and include, but are not limited to, biodiesels described in WO 07/061,903, U.S. Pat. No. 7,172,635, EP Pat. No. 1 227 143, WO 02/38709, WO 02/38707, and U.S. Publ. No. 2007/0113467. Transesterification of triglycerides in the oil produces long chain fatty acid esters, i.e., alkyl esters, and can be performed by any method known in the art for the production of biofuels. In some embodiments, the alkyl ether is a methyl ester or an ethyl ester. In some embodiments, the extracting the oil from the thraustochytrid biomass and transesterifying the oil can be performed simultaneously. In some embodiments, the extracting the oil from the thraustochytrid biomass and transesterifying the oil are performed separately. See, e.g., U.S. Publ. No. 2009/0064567.

In some embodiments, transesterification is performed using a lower alkyl alcohol and an acid or base catalyst. Alcohols suitable for use in the present invention include any lower alkyl alcohol containing from 1 to 6 carbon atoms (i.e., a C₁₋₆ alkyl alcohol, such as methyl, ethyl, isopropyl, butyl, pentyl, hexyl alcohols and isomers thereof). In some embodiments, the alcohol comprises from 5 wt. % to 70 wt. %, 5 wt. % to 60 wt. %, 5 wt. % to 50 wt. %, 7 wt. % to 40 wt. %, 9 wt. % to 30 wt. %, or 10 wt. % to 25 wt. % of the mixture of the oil composition, the alcohol, and a catalytic base. In some embodiments, the oil composition and the base can be added to either pure ethanol or pure methanol. The amount of alcohol used can vary with the solubility of the oil in the alcohol. Any base known in the art to be suitable for use as a reactant can be used, including bases of the formula RO-M, wherein M is a monovalent cation and RO is an alkoxide of a C₁₋₆ alkyl alcohol. Examples of suitable bases include, but are not limited to, elemental sodium, sodium methoxide, sodium ethoxide, potassium methoxide, and potassium ethoxide. In some embodiments, the base is sodium ethoxide. In some embodiments, the base is added to the reaction with the oil composition and the alcohol in an amount of from 0.05 to 2.0 molar equivalents of triglycerides in the oil, 0.05 to 1.5 molar equivalents, 0.1 to 1.4 molar equivalents, 0.2 to 1.3 molar equivalents, or 0.25 to 1.2 molar equivalents. The oil comprising triglycerides, the alcohol, and the base are reacted together at a temperature and for an amount of time that allows the production of an ester from the fatty acid residues and the alcohol. In some embodiments, the reacting of the composition in the presence of an alcohol and a base is performed at a temperature from 20° C. to 140° C., from 20° C. to 120° C., from 20° C. to 110° C., from 20° C. to 100° C., or from 20° C. to 90° C. In some embodiments, the reacting of the composition in the presence of an alcohol and a base is performed at a temperature of at least 20° C., at least 75° C., at least 80° C., at least 85° C., at least 90° C., at least 95° C., at least 105° C., or at least 120° C. In some embodiments, the reacting of the composition in the presence of an alcohol and a base is performed at a temperature of 20° C., 75° C., 80° C., 85° C., 90° C., 95° C., 105° C., or 120° C. In some embodiments, the reacting of the composition in the presence of an alcohol and a base is performed for a time from 2 hours to 36 hours, from 3 hours to 36 hours, from 4 hours to 36 hours, from 5 hours to 36 hours, or from 6 hours to 36 hours. In some embodiments, the reacting of the composition in the presence of an alcohol and a base is performed for 0.25, 0.5, 1, 2, 4, 5, 5.5, 6, 6.5, 7, 7.5, 8, 8.5, 10, 12, 16, 20, 24, 28, 32, or 36 hours. In some embodiments, the reacting of the oil composition, alcohol, and base can be conducted by refluxing the components to produce the fatty acid esters, such as PUFA esters. In some embodiments, the reacting of the oil composition can be carried out at a temperature that does not result in the refluxing of the reaction components. For example, carrying out the reacting of the oil composition under pressures greater than atmospheric pressure can increase the boiling point of the solvents present in the reaction mixture. Under such conditions, the reaction can occur at a temperature at which the solvents would boil at atmospheric pressure, but would not result in the refluxing of the reaction components. In some embodiments, the reaction is conducted at a pressure from 5 pounds per square inch (psi) to 20 psi; from 7 psi to 15 psi; or from 9 psi to 12 psi. In some embodiments, the reaction is conducted at a pressure of 7 psi, 8 psi, 9 psi, 10 psi, 11 psi, or 12 psi. Reactions conducted under pressure can be carried out at the reaction temperatures listed above. In some embodiments, reactions conducted under pressure can be carried out at least from 20° C. to 140° C., from 20° C. to 120° C., from 20° C. to 110° C., from 20° C. to 100° C., or from 20° C. to 90° C. In some embodiments, reactions conducted under pressure can be carried out at least 70° C., at least 75° C., at least 80° C., at least 85° C., or at least 90° C. In some embodiments, reactions conducted under pressure can be carried out at 70° C., 75° C., 80° C., 85° C., or 90° C.

Reduced amounts of PUFA in oil used for producing a biofuel, such as a biodiesel, can increase the energy density of the biofuel and can reduce the number of sites for potential oxidation or sulfation. In some embodiments, the thraustochytrid biomass or microbial oil, or triglyceride fraction thereof, comprises a greater percentage of monounsaturated fatty acids than polyunsaturated fatty acids. In some embodiments, the microbial oil is fractionated to remove polyunsaturated fatty acids. In some embodiments, the oil is hydrogenated in order to convert polyunsaturated fatty acids to monounsaturated fatty acids. To ensure that greater percentages of microalgal oil-derived biodiesel can be burned, any method of partial or total oil hydrogenation, as is routine in the manufacture of margarines, can be used. In some embodiments, thraustochytrids that produce low levels of PUFAs are used to produce the microbial oils. In some embodiments, thraustochytrids of the invention are selected that produce greater amounts of monounsaturated fatty acids than polyunsaturated fatty acids. In some embodiments, less than 50% of unsaturated fatty acids in the biological oil are PUFAs. In some embodiments, the unsaturated fatty acids in the biological oil contain less than 40%, less than 30%, less than 20%, less than 10%, or less than 5% PUFAs. In some embodiments, the microbial oil comprises less than 50%, less than 40%, less than 30%, less than 20%, less than 10%, or less than 5% by weight of PUFAs.

In addition to the transesterification methods described above, other techniques of reducing the viscosity of the microbial oils can also be used to produce biofuels. These techniques include, but are not limited to, the use of lipases, supercritical methanol catalysis, and the use of whole-cell systems involving cytoplasmic overexpression of lipases in a host cell followed by permeabilization of the host to allow catalysis of transesterification of triglycerides within the cytoplasm. In some embodiments, a thraustochytrid cell of the invention also comprises a polynucleotide sequence encoding a lipase for catalysis of transesterification of triglycerides within the cytoplasm. See, for example, U.S. Pat. No. 7,226,771, U.S. Publ. No. 2004/0005604, WO 03/089620, WO 05/086900, U.S. Publ. No. 2005/0108789, WO 05/032496, WO 05/108533, U.S. Pat. No. 6,982,155, WO 06/009676, WO 06/133698, WO 06/037334, WO 07/076,163, WO 07/056,786, and WO 06/124818, herein incorporated by reference in their entireties.

In some embodiments, the biofuel is produced by cracking the oil. In some embodiments, the biofuel is a jet biofuel. “Cracking” is understood in the art to describe the reduction of the chain length of fatty acids in an oil by methods such as those used in the oil industry. In some embodiments, the presence of a significant amount of polyunsaturated fatty acid in the oil will provide greater flexibility and variety for the production of hydrocarbons, since the multiple sites of unsaturation in a polyunsaturated fatty acid provide multiple sites for cleavage to make hydrocarbons. For example, certain jet fuels require hydrocarbons with two to eight carbons. Polyunsaturated fatty acids can be cleaved through known processes in the art, such as cracking, to produce shorter hydrocarbons of various chain lengths.

In some embodiments, the biofuel is produced by thermal depolymerization. In some embodiments, the biofuel is a renewable diesel. As used herein, thermal depolymerization includes any process for the production of renewable diesel using superheated water.

In some embodiments, the biofuel is produced by adding the oil to a petroleum refining process during the production of diesel fuel. In some embodiments, the biofuel is a co-processed renewable diesel.

Having generally described this invention, a further understanding can be obtained by reference to the examples provided herein. These examples are for purposes of illustration only and are not intended to be limiting.

### Example 1

### Construction of Xylose Transporter, Xylose Reductase, Xylulose Kinase, Xylitol Dehydrogenase, and Xylose Isomerase Expression Vectors

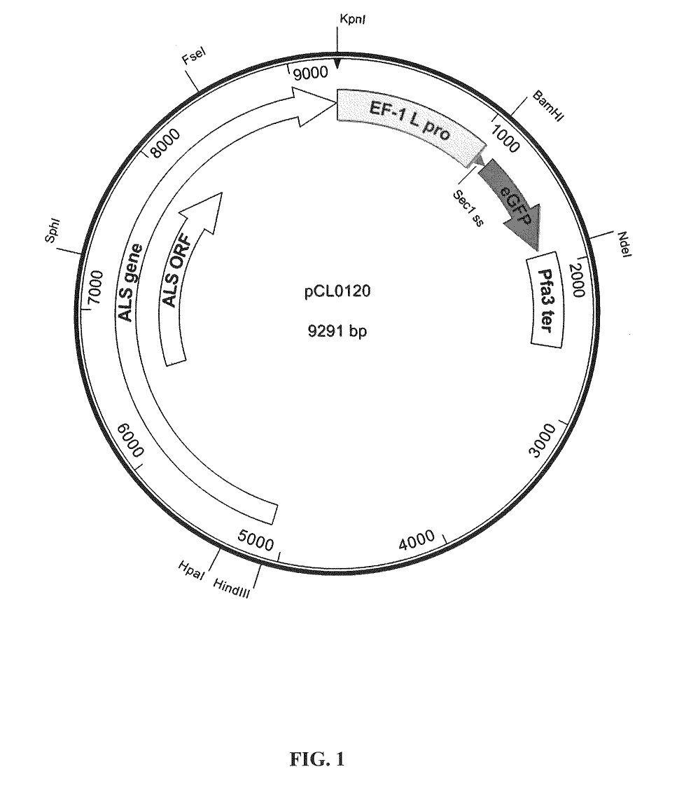

The vector pAB0018 (ATCC Accession No. PTA-9616) was digested with HindIII, treated with mung bean nuclease, purified, and then further digested with KpnI generating four fragments of various sizes. A fragment of 2552 bp was isolated by standard electrophoretic techniques in an agar gel and purified using commercial DNA purification kits. A second digest of pAB0018 with PmeI and KpnI was then performed. A fragment of 6732 bp was isolated and purified from this digest and ligated to the 2552 bp fragment. The ligation product was then used to transform commercially supplied strains of competent DH5-α E. coli cells (Invitrogen) using the manufacturer's protocol. Plasmids from ampicillin-resistant clones were propagated, purified, and then screened by restriction digests or PCR to confirm that the ligation generated the expected plasmid structures. One verified plasmid was designated pCL0120. See FIG. 1 and SEQ ID NO:12.

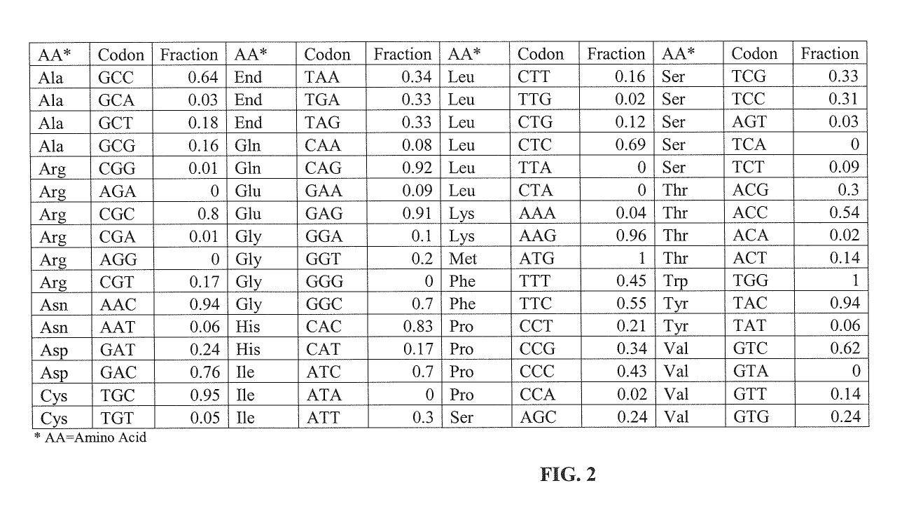

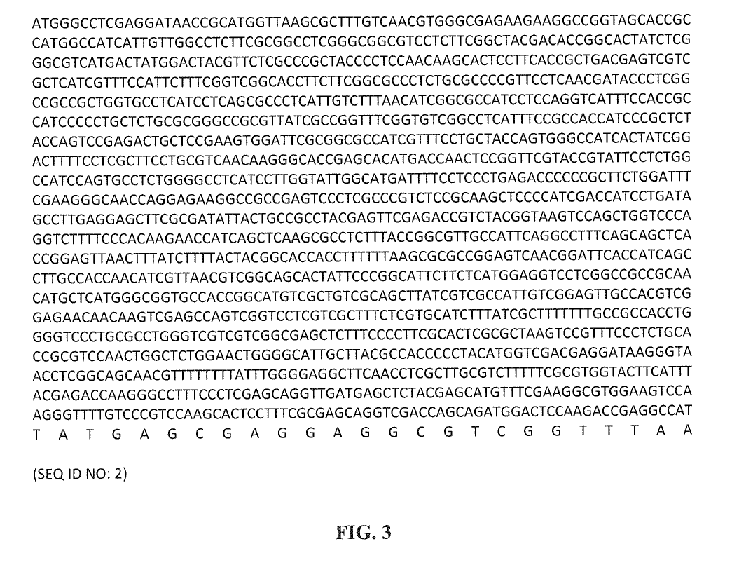

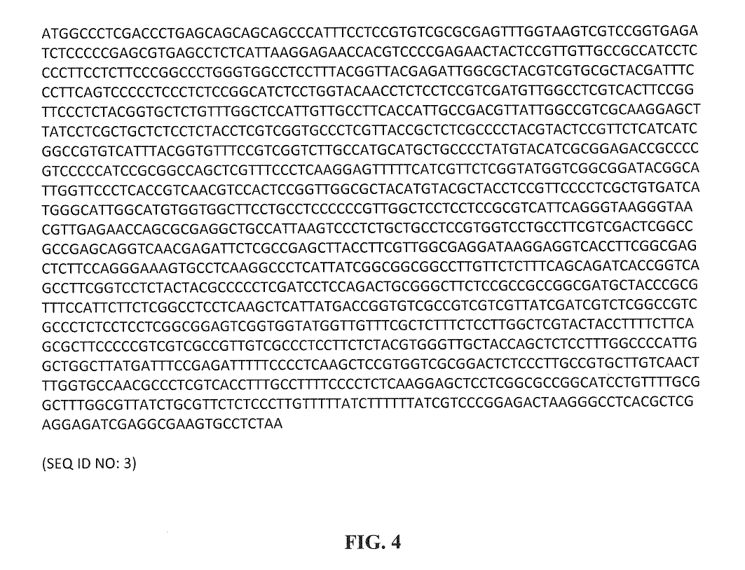

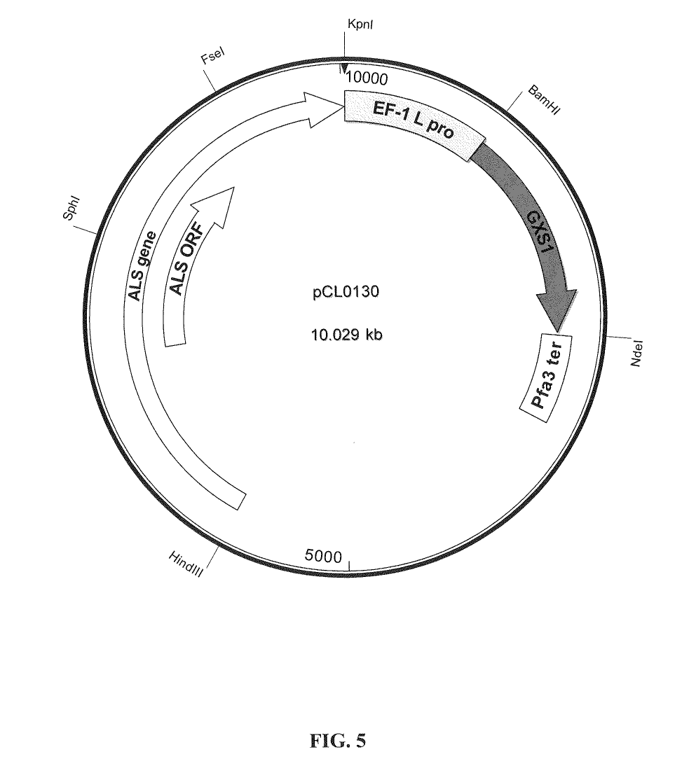

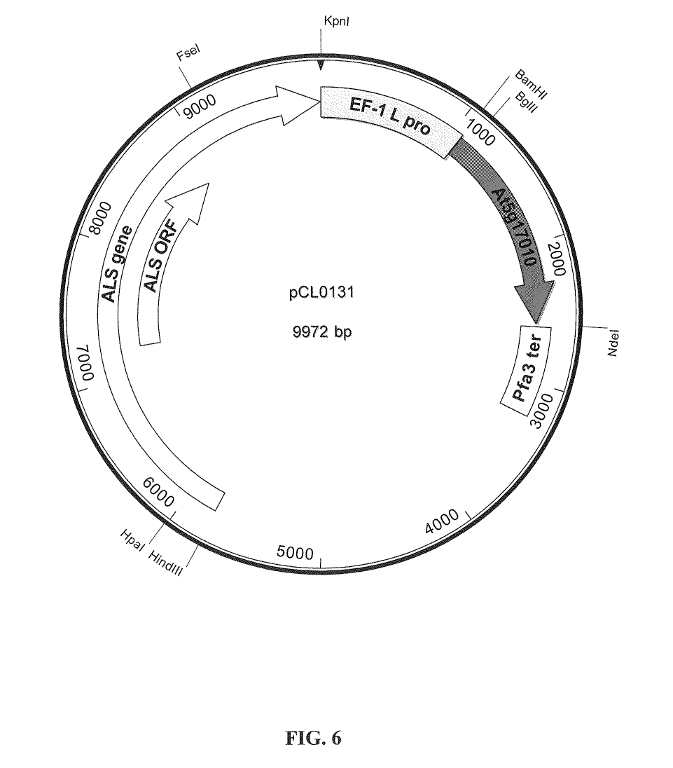

Sequences encoding the Candida intermedia xylose transporter protein GXS1 (GenBank Accession No. AJ875406) and the Arabidopsis thaliana xylose transporter protein At5g17010 (GenBank Accession No. BT015128) were sent to Blue Heron Biotechnology (Bothell, Wash.) for codon optimization and synthesis as guided by the Schizochytrium codon usage table shown in FIG. 2. SEQ ID NO:2 is the codon-optimized nucleic acid sequence of GSX1 (FIG. 3), while SEQ ID NO:3 is the codon-optimized nucleic acid sequence of At5g17010 (FIG. 4). SEQ ID NO:2 and SEQ ID NO:3 were respectively cloned into pCL0120 using the 5′ and 3′ restriction sites BamHI and NdeI for insertion and ligation according to standard techniques. Maps of the resulting vectors, pCL0130 and pCL0131 are shown in FIG. 5 and FIG. 6, respectively. Sequences for the vectors pCL0130 and pCL0131 are provided as SEQ ID NOs: 14 and 15, respectively.

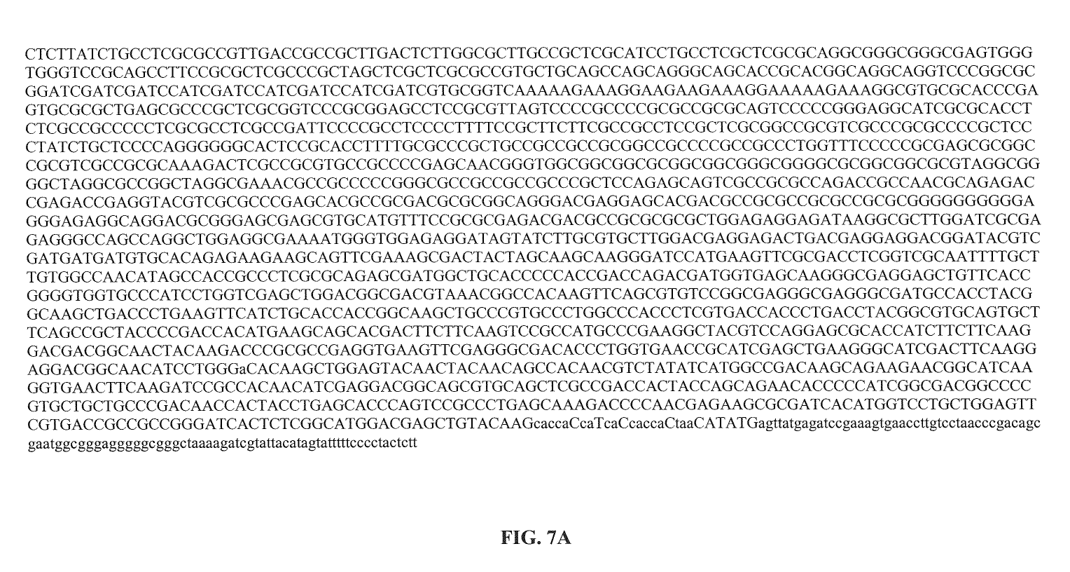

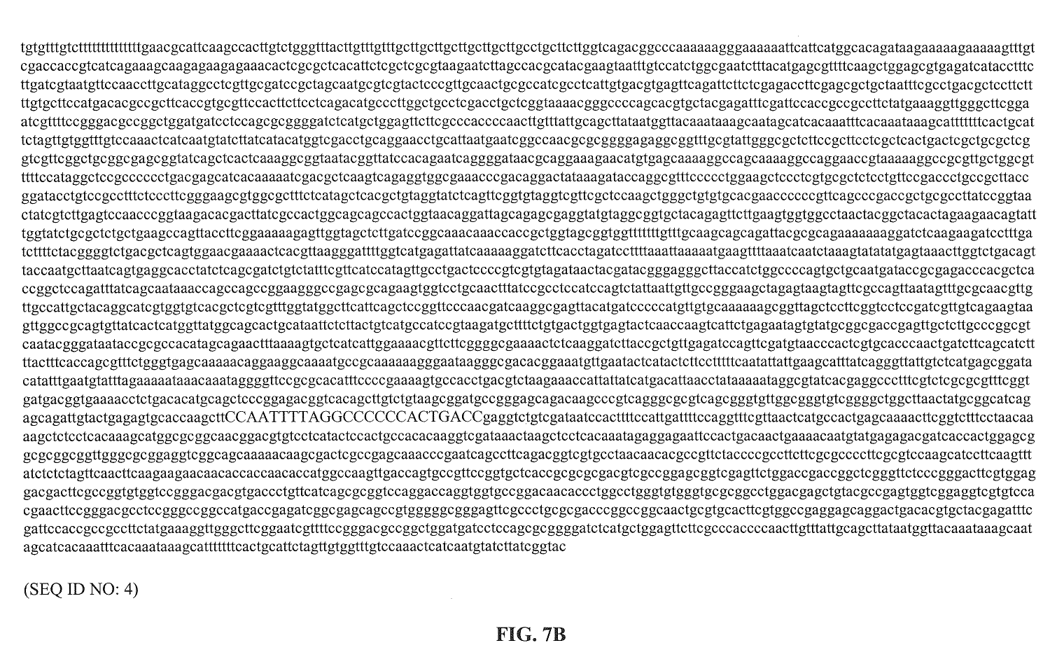

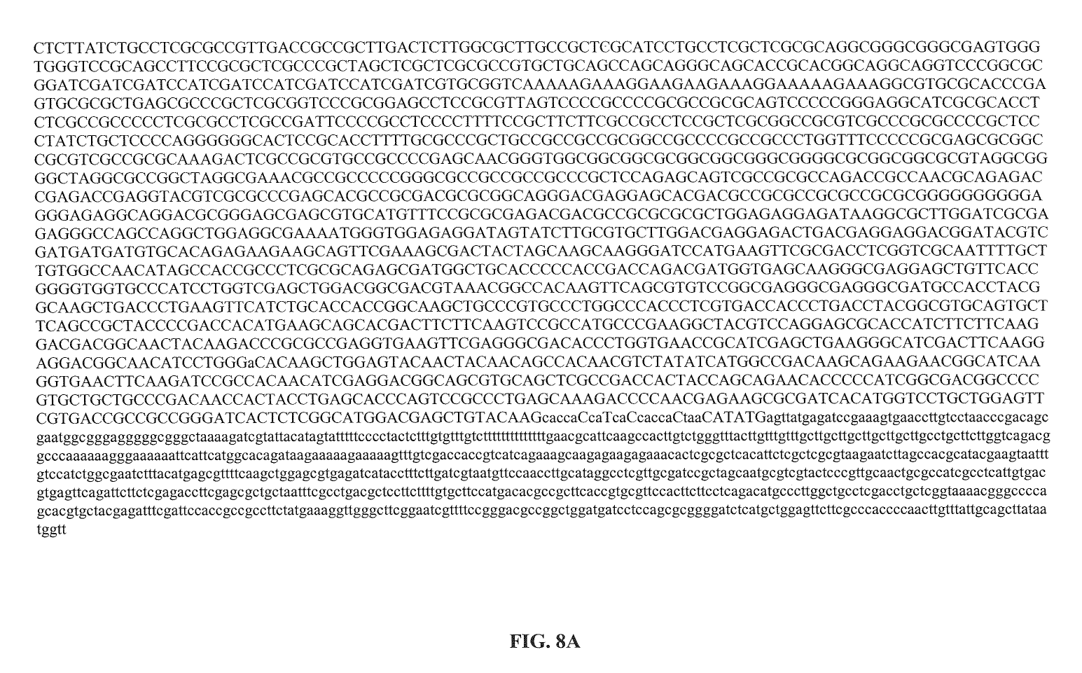

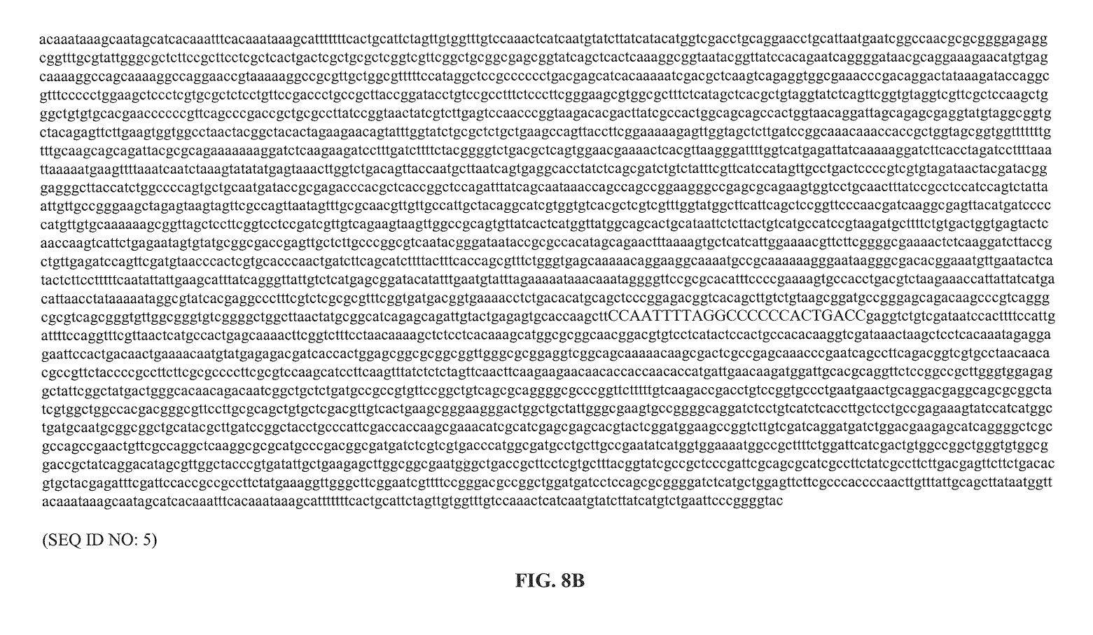

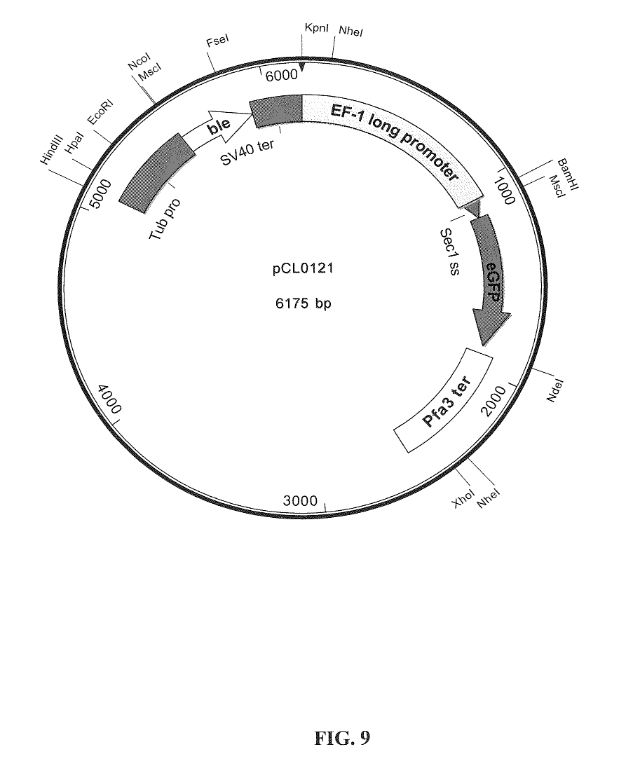

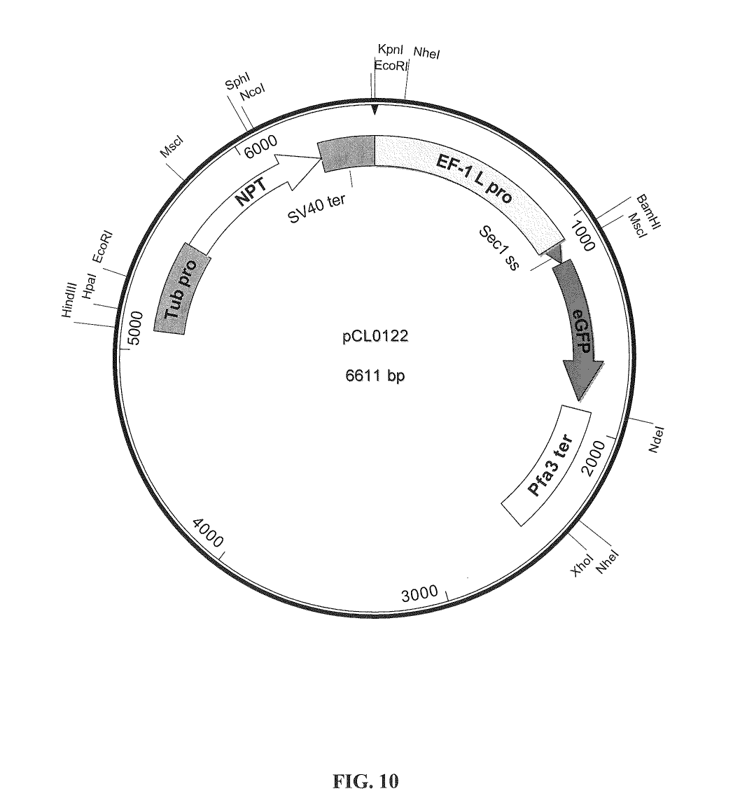

Vectors pCL0121 and pCL0122 were created by ligating a 5095 bp fragment which had been liberated from pCL0120 by digestion with HindIII and KpnI to synthetic selectable marker cassettes designed to confer resistance to either ZEOCIN™ or paromomycin. These cassettes were comprised of an alpha tubulin promoter to drive expression of either the sh ble gene (for ZEOCIN™) or the npt gene (for paromomycin). The transcripts of both selectable marker genes were terminated by an SV40 terminator. The full sequence of vectors pCL0121 (SEQ ID NO:4) and pCL0122 (SEQ ID NO:5) are shown in FIGS. 7 and 8, respectively. Maps of vectors pCL0121 and pCL0122 are shown in FIGS. 9 and 10, respectively.

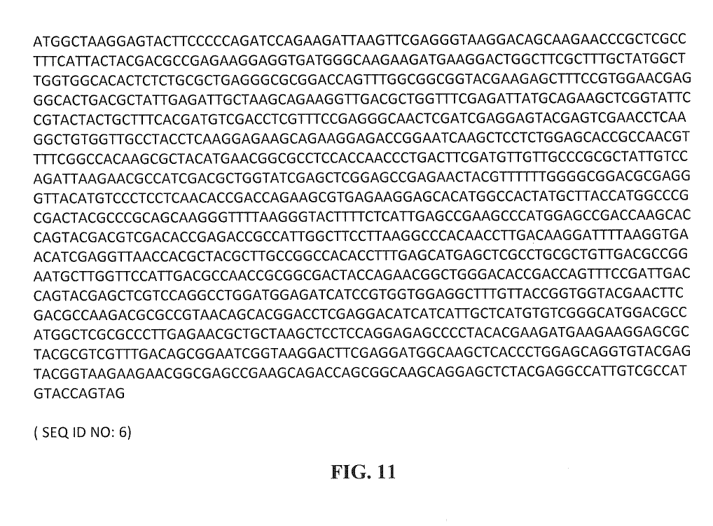

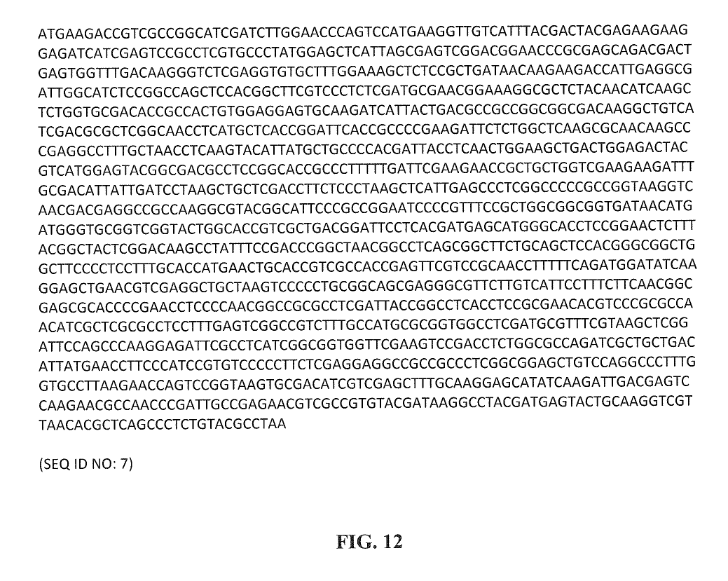

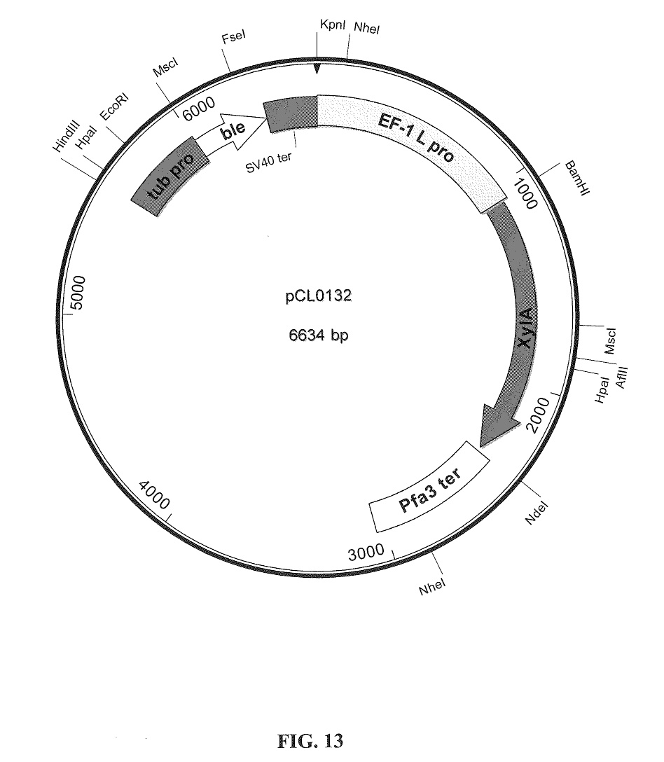

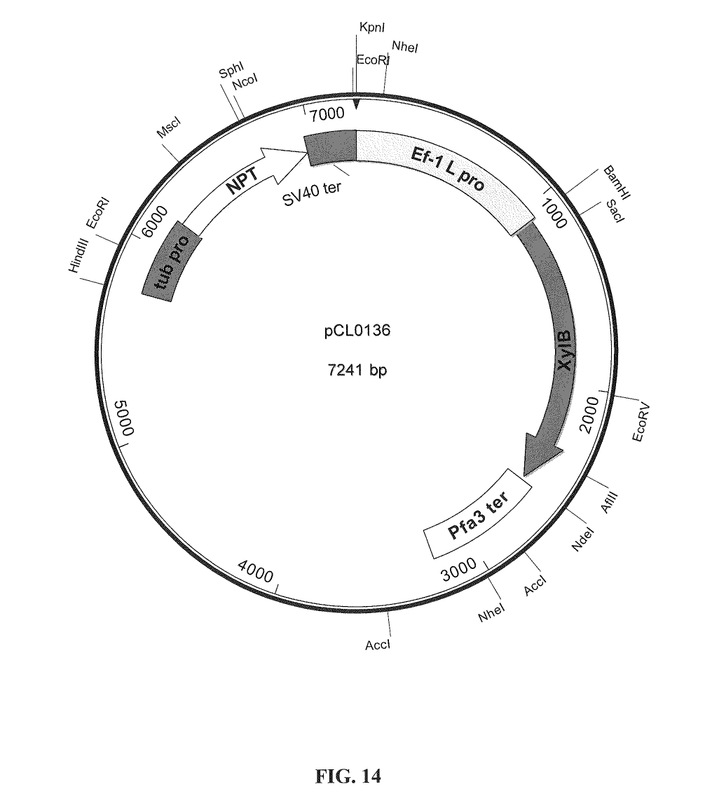

Sequences encoding the Piromyces sp. E2 xylose isomerase (CAB76571) and Piromyces sp. E2 xylulose kinase (AJ249910) were sent to Blue Heron Biotechnology (Bothell, Wash.) for codon optimization and synthesis as guided by the Schizochytrium codon usage table shown in FIG. 2. “XylA” (SEQ ID NO:6) is the codon-optimized nucleic acid sequence of CAB76571 (FIG. 11), while “XylB” (SEQ ID NO:7) is the codon-optimized nucleic acid sequence of AJ249910 (FIG. 12). SEQ ID NO:6 was cloned into the vector pCL0121 resulting in the vector designated pCL0132 (FIG. 13 and SEQ ID NO:16). SEQ ID NO:7 was cloned into the vector pCL0122 resulting in the vector designated pCL0136 (FIG. 14 and SEQ ID NO:20).

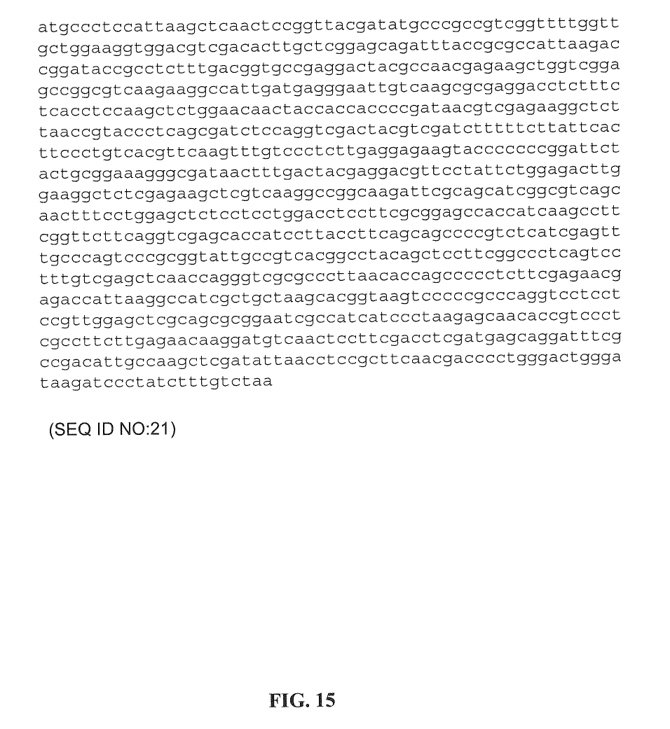

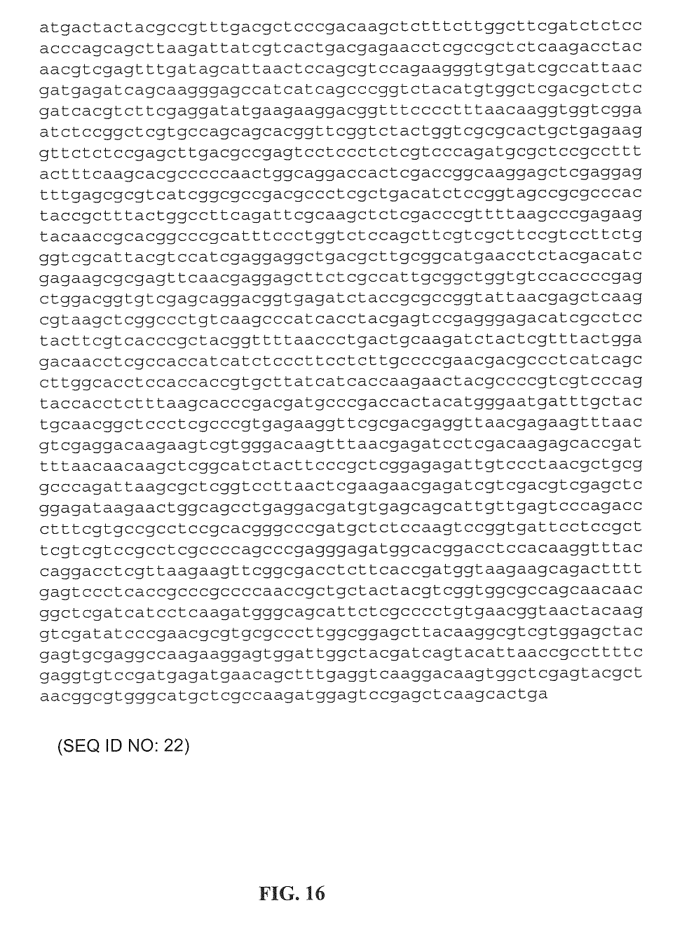

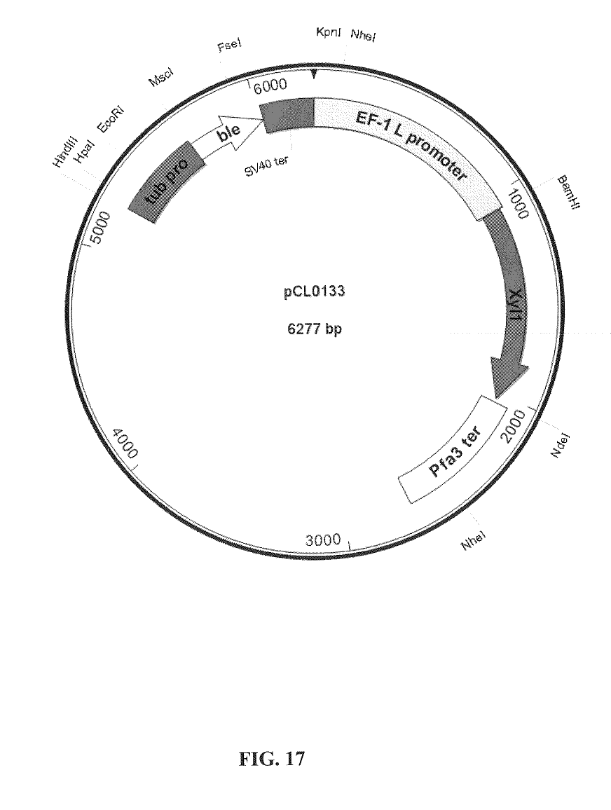

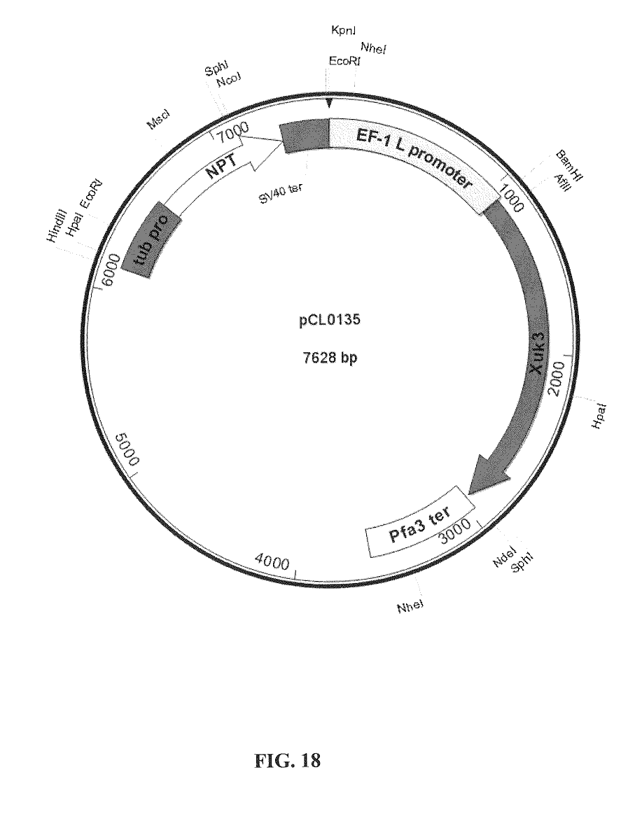

Sequences encoding the Pichia stipitis xylose reductase (X59465) and Pichia stipitis xylulose kinase (AF127802) were sent to Blue Heron Biotechnology (Bothell, Wash.) for codon optimization and synthesis as guided by the Schizochytrium codon usage table shown in FIG. 2. “Xyl1” (SEQ ID NO:21) is the codon-optimized nucleic acid sequence of X59465 (FIG. 15), while “Xuk3” (SEQ ID NO:22) is the codon-optimized nucleic acid sequence of AF127802 (FIG. 16). SEQ ID NO:21 was cloned into the vector pCL0121 resulting in the vector designated pCL0133 (FIG. 17 and SEQ ID NO:17). SEQ ID NO:22 was cloned into the vector pCL0122, resulting in the vector designated pCL0135 (FIG. 18 and SEQ ID NO:19).

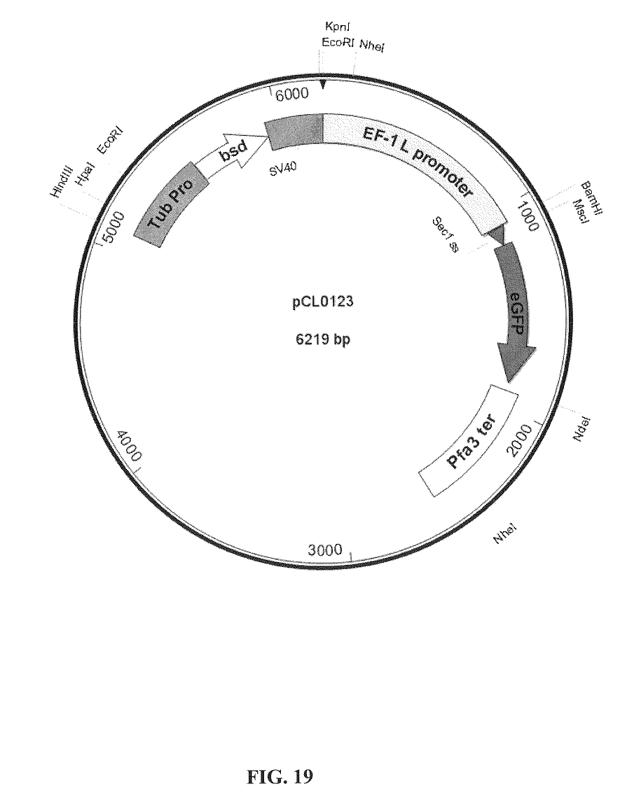

Vector pCL0123 was created by ligating a 5095 bp fragment which had been liberated from pCL0120 by digestion with HindIII and KpnI to a synthetic selectable marker cassette designed to confer resistance to ZEOCIN™. The cassette was comprised of an alpha tubulin promoter to drive expression of the sh ble gene (for ZEOCIN™). The transcript of the selectable marker gene was terminated by an SV40 terminator. A map of vector pCL0123 is shown in FIG. 19 and SEQ ID NO:13.

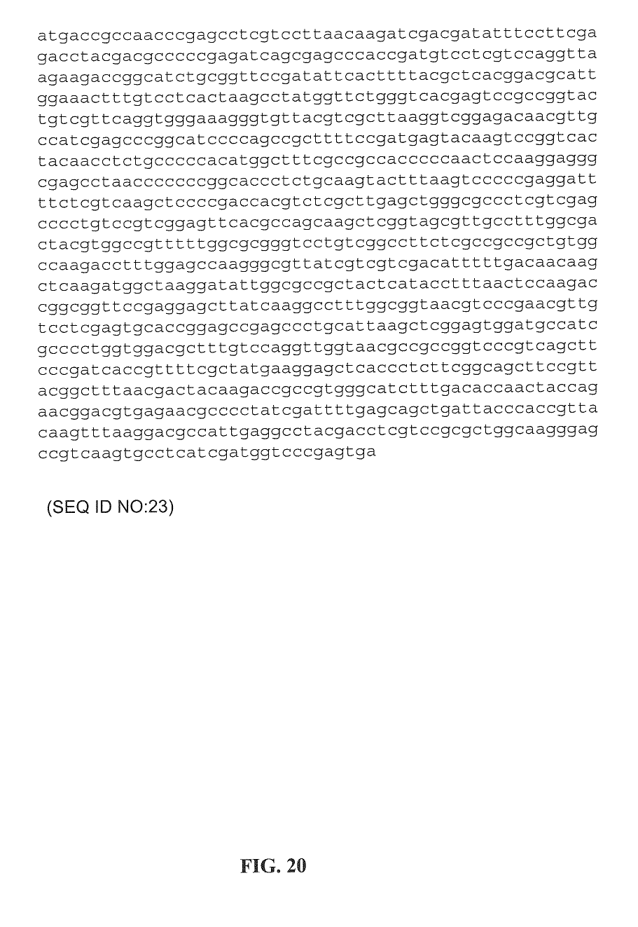

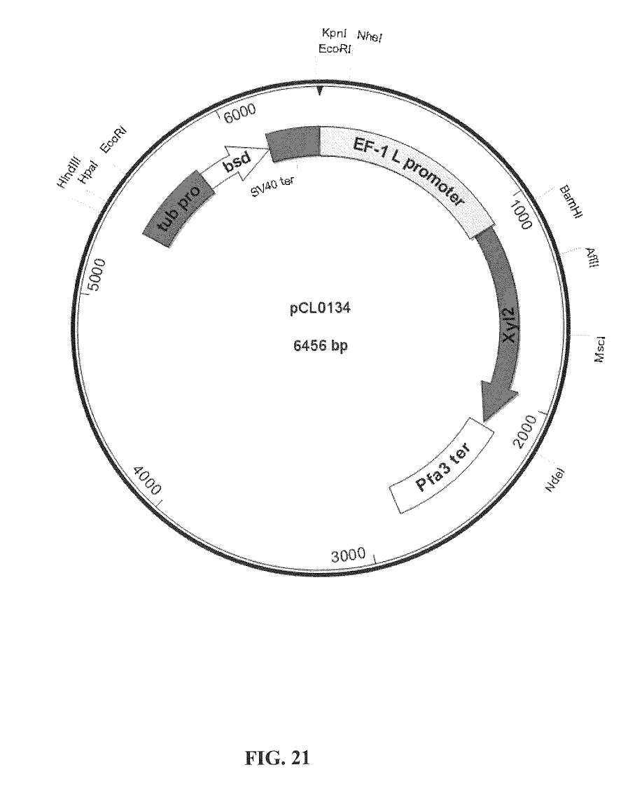

Sequences encoding the Pichia stipitis Xylitol dehydrogenase (X55392) was sent to Blue Heron Biotechnology (Bothell, Wash.) for codon optimization and synthesis as guided by the Schizochytrium codon usage table shown in FIG. 2. “Xyl2” (SEQ ID NO:23) is the codon-optimized nucleic acid sequence of X55392 (FIG. 20). SEQ ID NO:23 was cloned into the vector pCL0123 resulting in the vector designated pCL0134 (FIG. 21 and SEQ ID NO:18).

### Example 2

Transformation of Schizochytrium with Xylose Transporter, Xylose Isomerase, Xylose Reductase, Xylitol Dehydrogenase, or Xylulose Kinase Expression Vectors

Schizochytrium wild type strain ATCC 20888 was individually transformed with vectors pCL0130 (Candida intermedia xylose transporter), pCL0131 (Arabidopsis thaliana xylose transporter), pCL0132 (Piromyces sp. E2 xylose isomerase), pCL0133 (Pichia stipitis xylose reductase), pCL0134 (Pichia stipitis Xylitol dehydrogenase), pCL0135 (Pichia stipitis xylulose kinase), or pCL0136 (Piromyces sp. E2 xylulose kinase) using electroporation with enzyme pretreatment as described below.

Electroporation with enzyme pretreatment—Cells were grown in 50 mL of M50-20 media (see U.S. Publ. No. 2008/0022422) on a shaker at 200 rpm for 2 days at 30° C. The cells were diluted at 1:100 into M2B media (see following paragraph) and grown overnight (16-24 h), attempting to reach mid-log phase growth (OD₆₀₀ of 1.5-2.5). The cells were centrifuged in a 50 mL conical tube for 5 min at about 3000×g. The supernatant was removed and the cells were resuspended in 1 M mannitol, pH 5.5, in a suitable volume to reach a final concentration of 2 OD₆₀₀ units. 5 mL of cells were aliquoted into a 25 mL shaker flask and amended with 10 mM CaCl₂ (1.0 M stock, filter sterilized) and 0.25 mg/mL Protease XIV (10 mg/mL stock, filter sterilized; Sigma-Aldrich, St. Louis, Mo.). Flasks were incubated on a shaker at 30° C. and about 100 rpm for 4 h. Cells were monitored under the microscope to determine the degree of protoplasting, with single cells desired. The cells were centrifuged for 5 min at about 2500×g in round-bottom tubes (i.e., 14 mL Falcon™ tubes, BD Biosciences, San Jose, Calif.). The supernatant was removed and the cells were gently resuspended with 5 mL of ice cold 10% glycerol. The cells were re-centrifuged for 5 min at about 2500×g in round-bottom tubes. The supernatant was removed and the cells were gently resuspended with 500 μL of ice cold 10% glycerol, using wide-bore pipette tips. 90 μL of cells were aliquoted into a prechilled electro-cuvette (Gene Pulser® cuvette—0.1 cm gap or 0.2 cm gap, Bio-Rad, Hercules, Calif.). 1 μg to 5 μg of DNA (in less than or equal to a 10 μL volume) was added to the cuvette, mixed gently with a pipette tip, and placed on ice for 5 min. Cells were electroporated at 200 ohms (resistance), 25 μL (capacitance), and either 250V (for 0.1 cm gap) or 500V (0.2 cm gap). 0.5 mL of M50-20 media was added immediately to the cuvette. The cells were then transferred to 4.5 mL of M50-20 media in a 25 mL shaker flask and incubated for 2-3 h at 30° C. and about 100 rpm on a shaker. The cells were centrifuged for 5 min at about 2500×g in round bottom tubes. The supernatant was removed and the cell pellet was resupended in 0.5 mL of M50-20 media. Cells were plated onto an appropriate number (2 to 5) of M2B plates with appropriate selection and incubated at 30° C.

M2B medium consisting of 10 g/L glucose, 0.8 g/L (NH₄)₂SO₄, 5 g/L Na₂SO₄, 2 g/L MgSO₄.7H₂O, 0.5 g/L KH₂PO₄, 0.5 g/L KCl, 0.1 g/L CaCl₂.2H₂O, 0.1 M MES (pH 6.0) 0.1% PB26 metals, and 0.1% PB26 Vitamins (v/v). PB26 vitamins consisted of 50 mg/mL vitamin B12, 100 μg/mL thiamine, and 100 μg/mL Ca-pantothenate. PB26 metals were adjusted to pH 4.5 and consisted of 3 g/L FeSO₄.7H₂O, 1 g/L MnCl₂.₄H₂O, 800 mg/mL ZnSO₄H₂O, 20 mg/mL CoCl₂H₂O, 10 mg/mL Na₂MoO₄.2H₂O, 600 mg/mL CuSO₄.5H₂O, and 800 mg/mL NiSO₄.6H₂O. PB26 stock solutions were filter sterilized separately and added to the broth after autoclaving. Glucose, KH₂PO₄, and CaCl₂.2H₂O were each autoclaved separately from the remainder of the broth ingredients before mixing to prevent salt precipitation and carbohydrate caramelizing. All medium ingredients were purchased from Sigma Chemical (St. Louis, Mo.).

The transformants were selected for growth on solid media containing the appropriate antibiotic. Between 20 and 100 primary transformants of each vector were re-plated to “xylose-SSFM” solid media which is the same as SSFM (described below) except that it contains xylose instead of glucose as a sole carbon source, and no antibiotic were added. No growth was observed for any clones resulting from transformation with any individual vector under these conditions.

SSFM media: 50 g/L glucose, 13.6 g/L Na₂SO₄, 0.7 g/L K₂SO₄, 0.36 g/L KCl, 2.3 g/L MgSO₄.7H₂O, 0.1M MES (pH 6.0), 1.2 g/L (NH₄)₂SO₄, 0.13 g/L monosodium glutamate, 0.056 g/L KH₂PO₄, and 0.2 g/L CaCl₂.2H₂O. Vitamins were added at 1 mL/L from a stock consisting of 0.16 g/L vitamin B12, 9.7 g/L thiamine, and 3.3 g/L Ca-pantothenate. Trace metals were added at 2 mL/L from a stock consisting of 1 g/L citric acid, 5.2 g/L FeSO₄.7H₂O, 1.5 g/L MnCl₂.4H₂O, 1.5 g/L ZnSO₄.7H₂O, 0.02 g/L CoCl₂.6H₂O, 0.02 g/L Na₂MoO₄.2H₂O, 1.0 g/L CuSO₄.5H₂O, and 1.0 g/L NiSO₄.6H₂O, adjusted to pH 2.5.

gDNA from primary transformants of pCL0130 and pCL0131 was extracted and purified and used as a template for PCR to check for the presence of the transgene.

Genomic DNA Extraction Protocol for Schizochytrium—The Schizochytrium transformants were grown in 50 ml of media. 25 ml of culture was aseptically pipetted into a 50 ml conical vial and centrifuge for 4 minutes at 3000×g to form a pellet. The supernatant was removed and the pellet stored at −80° C. until use. The pellet was resuspended in approximately 4-5 volumes of a solution consisting of 20 mM Tris pH 8, 10 mM EDTA, 50 mM NaCl, 0.5% SDS and 100 ug/ml of Proteinase K in a 50 ml conical vial. The pellet was incubated at 50° C. with gentle rocking for 1 hour. Once lysed, 100 ug/ml of RNAse A was added and the solution was rocked for 10 minutes at 37° C. Next, 2 volumes of phenol:cholorform:isoamyl alcohol was added and the solution was rocked at room temperature for 1 hour and then centrifuged at 8000×g for 15 minutes. The supernatant was transferred into a clean tube. Again, 2 volumes of phenol:cholorform:isoamyl alcohol was added and the solution was rocked at room temperature for 1 hour and then centrifuged at 8000×g for 15 minutes and the supernatant was transferred into a clean tube. An equal volume of chloroform was added to the resulting supernatant and the solution was rocked at room temperature for 30 minutes. The solution was centrifuged at 8000×g for 15 minutes and the supernatant was transferred into a clean tube. An equal volume of chloroform was added to the resulting supernatant and the solution was rocked at room temperature for 30 minutes. The solution was centrifuged at 8000×g for 15 minutes and the supernatant was transferred into a clean tube. 0.3 volumes of 3M NaOAc and 2 volumes of 100% EtOH to were added to the supernatant, which was rocked gently for a few minutes. The DNA was spooled with a sterile glass rod and dip DNA into 70% EtOH for 1-2 minutes. The DNA was transferred into a 1.7 ml microfuge tube and allow to air dry for 10 minutes. Up to 0.5 ml of pre-warmed elution buffer (Tris buffer, 10 mM, pH 8.0) was added to the DNA and placed at 4° C. overnight.

Alternatively, after the RNAase incubation, the DNA was further purified using a Qiagen Genomic tip 500/G column (Qiagen, Inc USA, Valencia, Calif.), following the manufacturers protocol.

PCR—The primers used for detecting the GXS1 transgene were 5′CL0130 (CCTCGGGCGGCGTCCTCTT) (SEQ ID NO:8) and 3′CL0130 (GGCGGCCTTCTCCTGGTTGC) (SEQ ID NO:9). The primers used for detecting the At5g17010 transgene were 5′CL0131 (CTACTCCGTTGTTGCCGCCATCCT) (SEQ ID NO:10) and 3′CL0131 (CCGCCGACCATACCGAGAACGA) (SEQ ID NO:11). Of 10 clones resulting from the pCL0130 transformation, 4 were found to harbor the GSX1 transgene; and of 10 clones resulting from the pCL0131 transformation, 7 were found to harbor the At5g17010 transgene. One clone from each of these pCL0130 and pCL0131 positive transformants was selected for transformation with a second vector selected from pCL0132, pCL0133, pCL0134, pCL0135, or pCL0136 to result in the following combinations: pCL0130+pCL0132, pCL0130+pCL0133, pCL0130+pCL0134, pCL0130+pCL0135, pCL0130+pCL0136, pCL0131+pCL0132, pCL0131+pCL0133, pCL0131+pCL0134, pCL0131+pCL0135, and pCL0131+pCL0136. After transformation with the second vector, each culture was plated to SSFM with antibiotics or to xylose-SSFM directly. Of the many colonies which appeared after 4-12 days of growth on SSFM plates with antibiotics, 5 clones from each transformation were re-plated to xylose-SSFM. No growth was observed for any of the clones resulting from co-transformation of either pCL0130 or pCL0131 with any one of the other vectors on xylose-SSFM solid media plates.

### Example 3

### Expression of Xylose Transporters in Schizochytrium

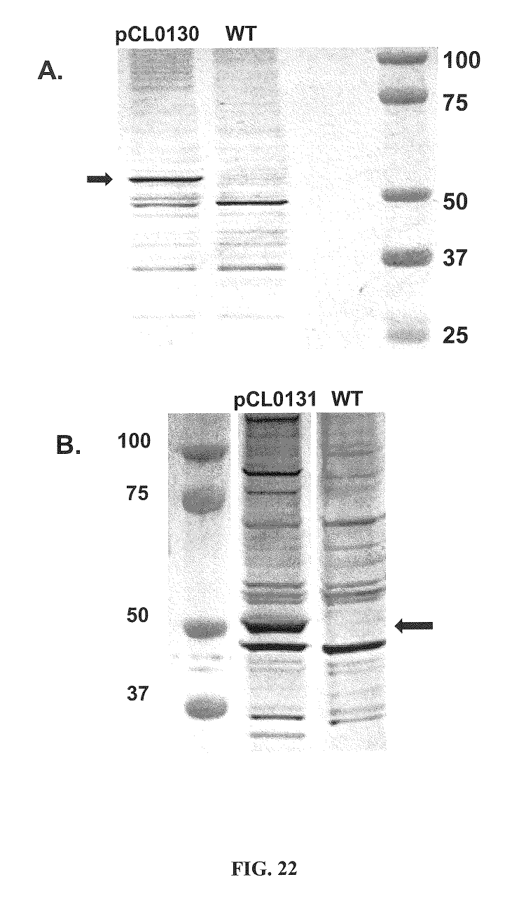

Supernatants of 50 mL shake-flask cultures grown in sPFM for 3 days were collected after cultures were centrifuged at 5000×g. sPFM media is described in Table 1, below. Culture pellets were lysed for extractions of proteins as previously described. See U.S. Publ. No. 2010/0233760. SDS-PAGE of culture pellet lysates was performed and gels were either stained directly with Coomassie dye or used for electrotransfer to PVDF membranes. After transfer, PVDF membranes were used for Western blotting according to common procedures. See, e.g., Sambrook et al. Briefly, membranes were probed with a 1:500 dilution of primary antisera derived from rabbits, which had been immunized by injection (Open Biosystems Products, Huntsville, Ala.) with a preparation of xylose transporter peptide conjugated to keyhole limpet hemocyanin (KLH). The specific synthetic peptides conjugated to KLH were RLRKLPIDHPDSLEELRD (SEQ ID NO:24)—“130-p1” or ETKGLTLEEIEAKCL (SEQ ID NO:25)—“131-p2 (Open Biosystems Products, Huntsville, Ala.) for generating antisera specific to either SEQ ID NO:2 (C. albicans GSX1) or SEQ ID NO:3 (A. thaliana At5g17010), respectively. The antisera was then followed by addition of a secondary, mouse anti-rabbit IgG monoclonal antibody conjugated to alkaline phosphatase (Promega Corporation, Madison, Wis.). BCIP/NBT reagent was then applied to the membrane to develop the signal. The Western blot of culture pellet lysates from a clone transformed with pCL0130 and wild type Schizochytrium strain ATCC 20888 are shown in FIG. 22 A. The Western blot of culture pellet lysates from a clone transformed with pCL0131 and wild type Schizochytrium strain ATCC 20888 are shown in FIG. 22 B. In each example, of all the bands in either wild type or transformant lysates, only one was found to be unique to a given transformant and in each case this unique band corresponded to the predicted size of the expressed xylose transporter.

TABLE 1
sPFM Media

| Component | Amount per liter | |
|---|---|---|
| Na₂SO₄ | 13.62 | g |
| K₂SO₄ | 0.72 | g |
| KCl | 0.56 | g |
| MgSO₄•7H₂O | 2.27 | g |
| (NH₄)2SO₄ | 3 | g |
| CaCl₂•2H₂O* | 0.19 | g |
| MSG | 3 | g |
| MES (100 mM) pH 6 | 21.4 | g |
| KH₂PO₄* | 0.4 | g |
| Glucose* | 50 | g |
| Vitamin B12* | 0.16 | mg |
| Thiamine* | 9.75 | mg |
| CaPantothenate* | 3.33 | mg |
| Citric Acid* | 2 | mg |
| FeSO₄•7H₂O* | 10.3 | mg |
| MnCl₂•4H₂O* | 3.1 | mg |
| ZnSO₄•7H₂O* | 1.93 | mg |
| CoCl₂•6H₂O* | 0.04 | mg |
| Na₂MoO₄•2H₂O* | 0.04 | mg |
| CuSO₄•5H₂O* | 2.07 | mg |
| NiSO₄•6H₂O* | 2.07 | mg |

pH to 2.5 with HCl
*Added after autoclaving.

### Example 4

Growth on Xylose of Schizochytrium Transformed with Xylose Transporter, Xylose Isomerase, and Xylulose Kinase Expression Vectors

Co-transformations of Schizochytrium wild type strain ATCC 20888 were performed with two different mixtures of vectors. The mixtures contained either the Candida intermedia xylose transporter (pCL0130) or the Arabidopsis thaliana xylose transporter (pCL0131) with two additional vectors: a vector containing Piromyces sp. E2 xylose isomerase (pCL0132) and a vector containing Piromyces sp. E2 xylose kinase (pCL0136). One combination of vectors, termed the 130 Piromyces Series, included pCL0130, pCL0132, and pCL0136. Another combination of vectors, termed the 131 Piromyces Series, included pCL0131, pCL0132, and pCL0136. Transformants were plated directly on solid xylose SSFM media and after 3-5 weeks colonies were picked and further propagated in liquid xylose-SSFM. Two colonies were recovered from the 130 Piromyces Series transformations and four colonies from the 131 Piromyces Series transformations. Several rounds of serial transfers in xylose-containing liquid media improved growth rates of the transformants. Transformants of the 130 Piromyces Series and the 131 Piromyces Series were also replated to solid SSFM media containing either sulfometum methyl (SMM), ZEOCIN™, or paromomycin. All transformants plated to these media were resistant to each antibiotic tested, indicating that transformants harbored all three of their respective vectors. Schizochytrium cells transformed with a xylose transporter, a xylose isomerase, and a xylulose kinase were able to grow in solid and liquid SSFM media containing xylose as a sole carbon source.

One clone from the Piromyces 130 Series and one clone from the Piromyces 131 Series were serially passaged in xylose-containing liquid media. Values for dry cell weight (DCW), fat as a weight percent of DCW (% Fat), and grams of fat per liter of cell culture (g/L Fat) for each culture were measured at serial passage numbers 5, 11, and 19. Results are shown in Table 2, below.

TABLE 2
Dry Cell Weight, % Fat, and g/L Fat for a Piromyces
130 Series and a Piromyces 131 Series Transformant
Grown in Xylose-Containing Media

| | |
|---|---|
| Piromyces 130 series | Piromyces 131 series |

Dry Cell Weight (DCW) (mg)

| | | |
|---|---|---|
| Passage 5 | 0.12 | 0.13 |
| Passage 11 | 0.36 | 0.26 |
| Passage 19 | 0.33 | 0.33 |

% Fat

| | | |
|---|---|---|
| Passage 5 | nd | nd |
| Passage 11 | 38.03 | 39.53 |
| Passage 19 | 43.30 | 50.50 |

g/L Fat

| | | |
|---|---|---|
| Passage 5 | nd | nd |
| Passage 12 | 2.74 | 2.06 |
| Passage 19 | 2.85 | 3.38 |

nd = not determined

The same clones were further grown in liquid culture for FAME analysis. Colonies picked from solid media plates selective for growth on xylose as a carbon source were propagated in liquid media containing xylose as the primary carbon source over 19 serial transfers. The inoculum used to start the 19th serial passage was also used to begin a comparative control culture for each clone in liquid media containing glucose as the primary carbon source. Wild type (WT) Schizochytrium was also grown in parallel in glucose-containing liquid medium. No growth of the wild type Schizochytrium strain was detected in xylose-containing liquid medium and thus FAME analysis and other growth characterization was not possible.

Oils were extracted from cell cultures using standard procedures and analyzed for fatty acid composition as percent of total fatty acid methyl esters (FAMEs). See, e.g., U.S. Publ. No. 2010/0239533, incorporated by reference herein in its entirety.

Dry cell weight (DCW), fat as a weight % of DCW (% Fat), grams of fat per liter of cell culture (g/L Fat), milligrams of DHA per gram of total fat, percent values of each fat detected, and summations of percent values of fat classes (saturates, monounsaturates, and fats of greater than 18 carbons in length) are listed in Table 3, below.

TABLE 3
FAME Analysis from a Piromyces 130 Series
and a Piromyces 131 Series Transformant

| | WT | 130 Series | 130 Series | 131 Series | 131 Series |
|---|---|---|---|---|---|
| | grown on | grown on | grown on | grown on | grown on |
| | glucose | glucose | xylose | glucose | xylose |
| DCW mg | 0.82 | 0.80 | 0.33 | 0.73 | 0.33 |
| DCW g/L | 16.38 | 15.95 | 6.59 | 14.61 | 6.68 |
| % Fat | 60.58 | 65.88 | 43.34 | 60.39 | 50.53 |
| g/L fat | 9.92 | 10.51 | 2.85 | 8.82 | 3.38 |
| Fatty Acid | | | | | |
| Profile: | | | | | |
| % 12:0 | 0.24 | 0.32 | 0.08 | 0.30 | 0.00 |
| % 12:1 | 0.00 | 0.00 | 0.00 | 0.00 | 0.00 |
| % 13:0 | 0.00 | 0.00 | 0.00 | 0.00 | 0.00 |
| % 13:1 | 0.00 | 0.00 | 0.00 | 0.00 | 0.00 |
| % 14:0 | 10.65 | 12.04 | 6.82 | 12.73 | 6.90 |
| % 14:1 | 0.00 | 0.00 | 0.00 | 0.00 | 0.00 |
| % 15:1 | 0.25 | 0.29 | 0.32 | 0.35 | 0.00 |
| % 16:0 | 34.43 | 29.40 | 25.43 | 30.27 | 32.48 |
| % 16:1 | 9.24 | 13.11 | 8.32 | 11.82 | 6.57 |
| % 16:2 | 0.00 | 0.00 | 0.00 | 0.00 | 0.00 |
| % 16:3 | 0.00 | 0.00 | 0.00 | 0.00 | 0.00 |
| % 17:0 | 0.00 | 0.00 | 0.00 | 0.00 | 0.00 |
| % 18:0 | 1.22 | 1.00 | 1.06 | 0.82 | 1.31 |
| % 18:1 n-9 | 0.00 | 0.00 | 0.00 | 0.00 | 0.00 |
| % 18:1 n-7 | 5.77 | 6.24 | 7.79 | 5.21 | 6.88 |
| % 18:2 | 0.00 | 0.00 | 0.00 | 0.00 | 0.00 |
| % 18:3 n-6 | 0.00 | 0.00 | 0.00 | 0.00 | 0.00 |
| % 18:3 n-3 | 0.00 | 0.00 | 0.15 | 0.00 | 0.00 |
| % 18:4 n-3 | 0.00 | 0.00 | 0.00 | 0.00 | 0.00 |
| % 20:0 | 0.00 | 0.00 | 0.00 | 0.00 | 0.00 |
| % 20:1 n-9 | 0.00 | 0.00 | 0.00 | 0.00 | 0.00 |
| % 20:2 | 0.00 | 0.00 | 0.00 | 0.00 | 0.00 |
| % 20:3 n-9 | 0.00 | 0.00 | 0.00 | 0.00 | 0.00 |
| % 20:3 n-6 | 0.00 | 0.00 | 0.00 | 0.00 | 0.00 |
| % 20:3 n-3 | 0.00 | 0.00 | 0.00 | 0.00 | 0.00 |
| % 20:4 ARA | 0.00 | 0.00 | 0.00 | 0.00 | 0.00 |
| % 20:5 n-3 | 0.00 | 0.00 | 1.58 | 0.61 | 2.09 |
| EPA | | | | | |
| % 22:0 | 0.00 | 0.00 | 0.00 | 0.00 | 0.00 |
| % 22:1 | 0.00 | 0.00 | 0.00 | 0.00 | 0.00 |
| % 22:2 | 0.00 | 0.00 | 0.00 | 0.00 | 0.00 |
| % 22:3 | 0.00 | 0.00 | 0.00 | 0.00 | 0.00 |
| % 22:4 n-6 | 0.00 | 0.00 | 0.00 | 0.00 | 0.00 |
| % 22:5 n-6 | 10.21 | 10.12 | 14.05 | 10.56 | 12.18 |
| % 22:5 n-3 | 0.00 | 0.00 | 0.30 | 0.00 | 0.00 |
| DPA | | | | | |
| % 22:6 n-3 | 27.98 | 27.48 | 33.58 | 26.87 | 31.59 |
| DHA | | | | | |
| % 24:0 | 0.00 | 0.00 | 0.00 | 0.00 | 0.00 |
| % 24:1 | 0.00 | 0.00 | 0.00 | 0.00 | 0.00 |
| % Unknown | 0.00 | 0.00 | 0.53 | 0.47 | 0.00 |
| mg/g DHA | 167.67 | 179.05 | 144.03 | 160.50 | 157.88 |
| Saturated | 45.32 | 41.76 | 32.34 | 43.30 | 39.38 |
| FAME | | | | | |
| Monoun- | 15.26 | 19.63 | 16.42 | 17.37 | 13.45 |
| saturated | | | | | |
| FAME | | | | | |
| >C18 FAME | 38.19 | 37.60 | 49.51 | 38.04 | 45.86 |

### Example 5

Growth on Xylose of Schizochytrium transformed with Xylose Transporter, Xylose Reductase, Xylitol Dehydrogenase, and Xylulose Kinase Expression Vectors

Co-transformations of Schizochytrium wild type strain ATCC 20888 were performed in which a vector containing the Candida intermedia xylose transporter (pCL0130) was co-transformed with three additional vectors: a vector containing Pichia stipitis xylose reductase (pCL0133), a vector containing Pichia stipitis Xylitol dehydrogenase (pCL0134), and a vector containing Pichia stipitis xylulose kinase (pCL0135). This combination of vectors was termed the 130 Pichia Series. Transformants were plated directly on solid xylose SSFM media and after 3-5 weeks colonies were picked and further propagated in liquid xylose-SSFM. Cotransformation rates for the 130 Pichia Series were similar to those for the 130 Piromyces Series and 131 Piromyces Series of Example 4. Schizochytrium cells transformed with a Candida intermedia xylose transporter, a xylose reductase, a xylitol dehydrogenase, and a xylulose kinase were able to grow in solid and liquid SSFM media containing xylose as a sole carbon source.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference.

1. A method of producing a thraustochytrid cell culture, comprising:
a. transforming a thraustochytrid cell with a nucleic acid molecule comprising a polynucleotide sequence encoding a polypeptide associated with xylose import, conversion of xylose to xylulose, phosphorylation of xylulose, or a combination thereof; and
b. growing the transformed thraustochytrid cell in a culture medium comprising xylose as a carbon source.

2. The method of claim 1, wherein the polypeptide associated with xylose import, conversion of xylose to xylulose, or phosphorylation of xylulose is selected from the group consisting of: a heterologous xylose transporter, a heterologous xylose isomerase, a heterologous xylulose kinase, a heterologous xylose reductase, a heterologous xylitol dehydrogenase, and combinations thereof.

3. The method of claim 1, wherein the thraustochytrid cell expresses a heterologous xylose transporter, a heterologous xylose isomerase, and a heterologous xylulose kinase.

4. The method of claim 1, wherein the thraustochytrid cell expresses a heterologous xylose transporter, a heterologous xylulose kinase, a heterologous xylose reductase, and a heterologous xylitol dehydrogenase.

5. The method of claim 1, wherein the polynucleotide sequence encoding the polypeptide associated with xylose import is at least 90% identical to a sequence selected from the group consisting of: the polynucleotide sequence of Accession No. AJ875406, BT015128, AF127802, AJ249910, X59465, or X55392; a polynucleotide sequence encoding the amino acid sequence of Accession No. CAB76571; the polynucleotide sequence of SEQ ID NO:2, SEQ ID NO:3, SEQ ID NO:6, SEQ ID NO:7, SEQ ID NO:21, SEQ ID NO:22, or SEQ ID NO:23; and combinations thereof.

6. The method of claim 1, wherein the thraustochytrid is a Schizochytrium or a Thraustochytrium.

7. A thraustochytrid cell culture produced by the method of claim 1.

8. A thraustochytrid cell comprising a nucleic acid molecule comprising a polynucleotide sequence encoding a polypeptide associated with xylose import, conversion of xylose to xylulose, phosphorylation of xylulose, or a combination thereof.

9. The cell of claim 8, wherein the polypeptide associated with xylose import, conversion of xylose to xylulose, or phosphorylation of xylulose is selected from the group consisting of: a heterologous xylose transporter, a heterologous xylose isomerase, a heterologous xylulose kinase, a heterologous xylose reductase, a heterologous xylitol dehydrogenase, and combinations thereof.

10. The cell of claim 8, wherein the wherein the thraustochytrid cell expresses a heterologous xylose transporter, a heterologous xylose isomerase, and a heterologous xylulose kinase.

11. The cell of claim 8, wherein the thraustochytrid cell expresses a heterologous xylose transporter, a heterologous xylulose kinase, a heterologous xylose reductase, and a heterologous xylitol dehydrogenase.

12. The cell of claim 8, wherein the polynucleotide sequence encoding the polypeptide associated with xylose import is at least 90% identical to a sequence selected from the group consisting of: the polynucleotide sequence of Accession No. AJ875406, BT015128, AF127802, AJ249910, X59465, or X55392; a polynucleotide sequence encoding the amino acid sequence of Accession No. CAB76571; the polynucleotide sequence of SEQ ID NO:2, SEQ ID NO:3, SEQ ID NO:6, SEQ ID NO:7, SEQ ID NO:21, SEQ ID NO:22, or SEQ ID NO:23; and combinations thereof.

13. The cell of claim 8, wherein the thraustochytrid is a Schizochytrium or a Thraustochytrium.

14. A thraustochytrid culture comprising:
a. the thraustochytrid cell of claim 8, and
b. a cell culture medium comprising xylose as a carbon source.

15. A method of producing a thraustochytrid biomass, comprising:
a. growing the thraustochytrid cell of claim 8 in a culture medium comprising xylose as a carbon source, and
b. harvesting the biomass from the culture medium.

16. A method of producing a microbial oil, comprising:
a. growing the thraustochytrid cell of claim 8 in a culture medium comprising xylose as a carbon source to produce a biomass, and
b. extracting an oil from the biomass.

17. A method of producing a food product, cosmetic, industrial composition, or pharmaceutical composition for an animal or human, comprising:
a. growing the thraustochytrid cell of claim 8 in a culture medium comprising xylose as a carbon source,
b. harvesting a biomass from the culture medium, and
c. preparing the food product, cosmetic, industrial composition, or pharmaceutical composition from the biomass.

18. The method of claim 17, further comprising extracting an oil from the biomass and preparing the food product, cosmetic, industrial composition, or pharmaceutical composition from the oil.

19. The method of claim 17, wherein the food product is an infant formula.

20. The method of claim 19, wherein the infant formula is suitable for premature infants.

21. The method of claim 17, wherein the food product is milk, a beverage, a therapeutic drink, a nutritional drink, or a combination thereof.

22. The method of claim 17, wherein the food product is an additive for animal or human food.

23. The method of claim 17, wherein the food product is a nutritional supplement.

24. The method of claim 17, wherein the food product is an animal feed.

25. The method of claim 24, wherein the animal feed is an aquaculture feed.

26. The method of claim 24, wherein the animal feed is a domestic animal feed, a zoological animal feed, a work animal feed, a livestock feed, or a combination thereof.

27. A method for producing a biofuel, comprising:
a. growing the thraustochytrid cell of claim 8 in a culture medium comprising xylose as a carbon source to produce a biomass,
b. extracting an oil from the biomass, and
c. producing a biofuel by transesterifying the oil, cracking the oil, processing the oil by thermal depolymerization, adding the oil to a petroleum refining process, or a combination thereof.

28. The method of claim 27, wherein the biofuel is produced by transesterifying the oil.

29. The method of claim 27, wherein the biofuel is a biodiesel.

30. The method of claim 29, wherein the biofuel is produced by cracking the oil.

31. The method of claim 27, wherein the biofuel is a jet biofuel.

32. The method of claim 27, wherein the biofuel is produced by processing the oil by thermal depolymerization.

33. The method of claim 27, wherein the biofuel is a renewable diesel.

34. The method of claim 27, wherein the biofuel is produced by adding the oil to a petroleum refining process.

35. The method of claim 27, wherein the biofuel is a co-processed renewable diesel.


SEQUENCE LISTING (25 sequences)

SEQ ID NO: 1 - DNA, 1614 bp, Artificial Sequence
  Secretion signal
ggatccatgaagttcgcgacctcggtcgcaattttgcttgtggccaacatagccaccgccctcgcgtcgatgaccaacgagacctcggaccgccctctcgtgcactttacccccaacaagggttggatgaacgatcccaacggcctctggtacgacgagaaggatgctaagtggcacctttactttcagtacaaccctaacgacaccgtctggggcaccccgctcttctggggccacgccacctccgacgacctcaccaactgggaggaccagcccattgctatcgcccccaagcgcaacgactcgggagctttttccggttccatggttgtggactacaacaacacctccggtttttttaacgacaccattgacccccgccagcgctgcgtcgccatctggacctacaacacgcccgagagcgaggagcagtacatcagctacagccttgatggaggctacacctttaccgagtaccagaagaaccctgtcctcgccgccaactccacccagttccgcgaccctaaggttttttggtacgagccttcccagaagtggattatgaccgccgctaagtcgcaggattacaagatcgagatctacagcagcgacgacctcaagtcctggaagcttgagtccgcctttgccaacgagggttttctcggataccagtacgagtgccccggtctcatcgaggtccccaccgagcaggacccgtccaagtcctactgggtcatgtttatttccatcaaccctggcgcccctgccggcggcagcttcaaccagtacttcgtcggctcctttaacggcacgcattttgaggccttcgacaaccagtcccgcgtcgtcgacttcggcaaggactactacgccctccagaccttctttaacaccgaccccacctacggcagcgccctcggtattgcttgggcctccaactgggagtactccgctttcgtccccactaacccctggcgcagctcgatgtccctcgtccgcaagttttcgcttaacaccgagtaccaggccaaccccgagaccgagcttattaacctgaaggccgagcctattctcaacatctccaacgctggcccctggtcccgctttgctactaacactaccctcaccaaggccaactcctacaacgtcgatctctccaactccaccggtactcttgagtttgagctcgtctacgccgtcaacaccacccagaccatctccaagtccgtcttcgccgacctctccctctggttcaagggccttgaggaccccgaggagtacctgcgcatgggttttgaggtctccgcctcctccttcttcctcgatcgcggtaactccaaggttaagtttgtcaaggagaacccctactttactaaccgtatgagcgtcaacaaccagccctttaagtccgagaacgatcttagctactacaaggtttacggcctcctcgaccagaacattctcgagctctactttaacgacggagatgtcgtcagcaccaacacctactttatgaccactggaaacgccctcggcagcgtgaacatgaccaccggagtcgacaacctcttttacattgacaagtttcaggttcgcgaggttaagtaacatatg

SEQ ID NO: 2 - DNA, 1569 bp, Candida intermedia
  xylose transporter protein GXS1, codon optimized
atgggcctcgaggataaccgcatggttaagcgctttgtcaacgtgggcgagaagaaggccggtagcaccgccatggccatcattgttggcctcttcgcggcctcgggcggcgtcctcttcggctacgacaccggcactatctcgggcgtcatgactatggactacgttctcgcccgctacccctccaacaagcactccttcaccgctgacgagtcgtcgctcatcgtttccattctttcggtcggcaccttcttcggcgccctctgcgccccgttcctcaacgataccctcggccgccgctggtgcctcatcctcagcgccctcattgtctttaacatcggcgccatcctccaggtcatttccaccgccatccccctgctctgcgcgggccgcgttatcgccggtttcggtgtcggcctcatttccgccaccatcccgctctaccagtccgagactgctccgaagtggattcgcggcgccatcgtttcctgctaccagtgggccatcactatcggacttttcctcgcttcctgcgtcaacaagggcaccgagcacatgaccaactccggttcgtaccgtattcctctggccatccagtgcctctggggcctcatccttggtattggcatgattttcctccctgagaccccccgcttctggatttcgaagggcaaccaggagaaggccgccgagtccctcgcccgtctccgcaagctccccatcgaccatcctgatagccttgaggagcttcgcgatattactgccgcctacgagttcgagaccgtctacggtaagtccagctggtcccaggtcttttcccacaagaaccatcagctcaagcgcctctttaccggcgttgccattcaggcctttcagcagctcaccggagttaactttatcttttactacggcaccaccttttttaagcgcgccggagtcaacggattcaccatcagccttgccaccaacatcgttaacgtcggcagcactattcccggcattcttctcatggaggtcctcggccgccgcaacatgctcatgggcggtgccaccggcatgtcgctgtcgcagcttatcgtcgccattgtcggagttgccacgtcggagaacaacaagtcgagccagtcggtcctcgtcgctttctcgtgcatctttatcgctttttttgccgccacctggggtccctgcgcctgggtcgtcgtcggcgagctctttccccttcgcactcgcgctaagtccgtttccctctgcaccgcgtccaactggctctggaactggggcattgcttacgccaccccctacatggtcgacgaggataagggtaacctcggcagcaacgttttttttatttggggaggcttcaacctcgcttgcgtctttttcgcgtggtacttcatttacgagaccaagggcctttccctcgagcaggttgatgagctctacgagcatgtttcgaaggcgtggaagtccaagggttttgtcccgtccaagcactcctttcgcgagcaggtcgaccagcagatggactccaagaccgaggccattatgagcgaggaggcgtcggtttaa

SEQ ID NO: 3 - DNA, 1512 bp, Arabidopsis thaliana
  xylose transporter protein At5g17010, codon optimized
atggccctcgaccctgagcagcagcagcccatttcctccgtgtcgcgcgagtttggtaagtcgtccggtgagatctcccccgagcgtgagcctctcattaaggagaaccacgtccccgagaactactccgttgttgccgccatcctccccttcctcttcccggccctgggtggcctcctttacggttacgagattggcgctacgtcgtgcgctacgatttcccttcagtccccctccctctccggcatctcctggtacaacctctcctccgtcgatgttggcctcgtcacttccggttccctctacggtgctctgtttggctccattgttgccttcaccattgccgacgttattggccgtcgcaaggagcttatcctcgctgctctcctctacctcgtcggtgccctcgttaccgctctcgcccctacgtactccgttctcatcatcggccgtgtcatttacggtgtttccgtcggtcttgccatgcatgctgcccctatgtacatcgcggagaccgccccgtcccccatccgcggccagctcgtttccctcaaggagtttttcatcgttctcggtatggtcggcggatacggcattggttccctcaccgtcaacgtccactccggttggcgctacatgtacgctacctccgttcccctcgctgtgatcatgggcattggcatgtggtggcttcctgcctccccccgttggctcctcctccgcgtcattcagggtaagggtaacgttgagaaccagcgcgaggctgccattaagtccctctgctgcctccgtggtcctgccttcgtcgactcggccgccgagcaggtcaacgagattctcgccgagcttaccttcgttggcgaggataaggaggtcaccttcggcgagctcttccagggaaagtgcctcaaggccctcattatcggcggcggccttgttctctttcagcagatcaccggtcagccttcggtcctctactacgccccctcgatcctccagactgcgggcttctccgccgccggcgatgctacccgcgtttccattcttctcggcctcctcaagctcattatgaccggtgtcgccgtcgtcgttatcgatcgtctcggccgtcgccctctcctcctcggcggagtcggtggtatggttgtttcgctctttctccttggctcgtactaccttttcttcagcgcttcccccgtcgtcgccgttgtcgccctccttctctacgtgggttgctaccagctctcctttggccccattggctggcttatgatttccgagatttttcccctcaagctccgtggtcgcggactctcccttgccgtgcttgtcaactttggtgccaacgccctcgtcacctttgccttttcccctctcaaggagctcctcggcgccggcatcctgttttgcggctttggcgttatctgcgttctctcccttgtttttatcttttttatcgtcccggagactaagggcctcacgctcgaggagatcgaggcgaagtgcctctaa

SEQ ID NO: 4 - DNA, 6175 bp, Artificial Sequence
  vector pCL0121
ctcttatctgcctcgcgccgttgaccgccgcttgactcttggcgcttgccgctcgcatcctgcctcgctcgcgcaggcgggcgggcgagtgggtgggtccgcagccttccgcgctcgcccgctagctcgctcgcgccgtgctgcagccagcagggcagcaccgcacggcaggcaggtcccggcgcggatcgatcgatccatcgatccatcgatccatcgatcgtgcggtcaaaaagaaaggaagaagaaaggaaaaagaaaggcgtgcgcacccgagtgcgcgctgagcgcccgctcgcggtcccgcggagcctccgcgttagtccccgccccgcgccgcgcagtcccccgggaggcatcgcgcacctctcgccgccccctcgcgcctcgccgattccccgcctccccttttccgcttcttcgccgcctccgctcgcggccgcgtcgcccgcgccccgctccctatctgctccccaggggggcactccgcaccttttgcgcccgctgccgccgccgcggccgccccgccgccctggtttcccccgcgagcgcggccgcgtcgccgcgcaaagactcgccgcgtgccgccccgagcaacgggtggcggcggcgcggcggcgggcggggcgcggcggcgcgtaggcggggctaggcgccggctaggcgaaacgccgcccccgggcgccgccgccgcccgctccagagcagtcgccgcgccagaccgccaacgcagagaccgagaccgaggtacgtcgcgcccgagcacgccgcgacgcgcggcagggacgaggagcacgacgccgcgccgcgccgcgcggggggggggagggagaggcaggacgcgggagcgagcgtgcatgtttccgcgcgagacgacgccgcgcgcgctggagaggagataaggcgcttggatcgcgagagggccagccaggctggaggcgaaaatgggtggagaggatagtatcttgcgtgcttggacgaggagactgacgaggaggacggatacgtcgatgatgatgtgcacagagaagaagcagttcgaaagcgactactagcaagcaagggatccatgaagttcgcgacctcggtcgcaattttgcttgtggccaacatagccaccgccctcgcgcagagcgatggctgcacccccaccgaccagacgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctgggacacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcaccaccatcaccaccactaacatatgagttatgagatccgaaagtgaaccttgtcctaacccgacagcgaatggcgggagggggcgggctaaaagatcgtattacatagtatttttcccctactctttgtgtttgtcttttttttttttttgaacgcattcaagccacttgtctgggtttacttgtttgtttgcttgcttgcttgcttgcttgcctgcttcttggtcagacggcccaaaaaagggaaaaaattcattcatggcacagataagaaaaagaaaaagtttgtcgaccaccgtcatcagaaagcaagagaagagaaacactcgcgctcacattctcgctcgcgtaagaatcttagccacgcatacgaagtaatttgtccatctggcgaatctttacatgagcgttttcaagctggagcgtgagatcatacctttcttgatcgtaatgttccaaccttgcataggcctcgttgcgatccgctagcaatgcgtcgtactcccgttgcaactgcgccatcgcctcattgtgacgtgagttcagattcttctcgagaccttcgagcgctgctaatttcgcctgacgctccttcttttgtgcttccatgacacgccgcttcaccgtgcgttccacttcttcctcagacatgcccttggctgcctcgacctgctcggtaaaacgggccccagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatacatggtcgacctgcaggaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaagcttccaattttaggccccccactgaccgaggtctgtcgataatccacttttccattgattttccaggtttcgttaactcatgccactgagcaaaacttcggtctttcctaacaaaagctctcctcacaaagcatggcgcggcaacggacgtgtcctcatactccactgccacacaaggtcgataaactaagctcctcacaaatagaggagaattccactgacaactgaaaacaatgtatgagagacgatcaccactggagcggcgcggcggttgggcgcggaggtcggcagcaaaaacaagcgactcgccgagcaaacccgaatcagccttcagacggtcgtgcctaacaacacgccgttctaccccgccttcttcgcgccccttcgcgtccaagcatccttcaagtttatctctctagttcaacttcaagaagaacaacaccaccaacaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcggtac

SEQ ID NO: 5 - DNA, 6611 bp, Artificial Sequence
  vector pCL0122
ctcttatctgcctcgcgccgttgaccgccgcttgactcttggcgcttgccgctcgcatcctgcctcgctcgcgcaggcgggcgggcgagtgggtgggtccgcagccttccgcgctcgcccgctagctcgctcgcgccgtgctgcagccagcagggcagcaccgcacggcaggcaggtcccggcgcggatcgatcgatccatcgatccatcgatccatcgatcgtgcggtcaaaaagaaaggaagaagaaaggaaaaagaaaggcgtgcgcacccgagtgcgcgctgagcgcccgctcgcggtcccgcggagcctccgcgttagtccccgccccgcgccgcgcagtcccccgggaggcatcgcgcacctctcgccgccccctcgcgcctcgccgattccccgcctccccttttccgcttcttcgccgcctccgctcgcggccgcgtcgcccgcgccccgctccctatctgctccccaggggggcactccgcaccttttgcgcccgctgccgccgccgcggccgccccgccgccctggtttcccccgcgagcgcggccgcgtcgccgcgcaaagactcgccgcgtgccgccccgagcaacgggtggcggcggcgcggcggcgggcggggcgcggcggcgcgtaggcggggctaggcgccggctaggcgaaacgccgcccccgggcgccgccgccgcccgctccagagcagtcgccgcgccagaccgccaacgcagagaccgagaccgaggtacgtcgcgcccgagcacgccgcgacgcgcggcagggacgaggagcacgacgccgcgccgcgccgcgcggggggggggagggagaggcaggacgcgggagcgagcgtgcatgtttccgcgcgagacgacgccgcgcgcgctggagaggagataaggcgcttggatcgcgagagggccagccaggctggaggcgaaaatgggtggagaggatagtatcttgcgtgcttggacgaggagactgacgaggaggacggatacgtcgatgatgatgtgcacagagaagaagcagttcgaaagcgactactagcaagcaagggatccatgaagttcgcgacctcggtcgcaattttgcttgtggccaacatagccaccgccctcgcgcagagcgatggctgcacccccaccgaccagacgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctgggacacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcaccaccatcaccaccactaacatatgagttatgagatccgaaagtgaaccttgtcctaacccgacagcgaatggcgggagggggcgggctaaaagatcgtattacatagtatttttcccctactctttgtgtttgtcttttttttttttttgaacgcattcaagccacttgtctgggtttacttgtttgtttgcttgcttgcttgcttgcttgcctgcttcttggtcagacggcccaaaaaagggaaaaaattcattcatggcacagataagaaaaagaaaaagtttgtcgaccaccgtcatcagaaagcaagagaagagaaacactcgcgctcacattctcgctcgcgtaagaatcttagccacgcatacgaagtaatttgtccatctggcgaatctttacatgagcgttttcaagctggagcgtgagatcatacctttcttgatcgtaatgttccaaccttgcataggcctcgttgcgatccgctagcaatgcgtcgtactcccgttgcaactgcgccatcgcctcattgtgacgtgagttcagattcttctcgagaccttcgagcgctgctaatttcgcctgacgctccttcttttgtgcttccatgacacgccgcttcaccgtgcgttccacttcttcctcagacatgcccttggctgcctcgacctgctcggtaaaacgggccccagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatacatggtcgacctgcaggaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaagcttccaattttaggccccccactgaccgaggtctgtcgataatccacttttccattgattttccaggtttcgttaactcatgccactgagcaaaacttcggtctttcctaacaaaagctctcctcacaaagcatggcgcggcaacggacgtgtcctcatactccactgccacacaaggtcgataaactaagctcctcacaaatagaggagaattccactgacaactgaaaacaatgtatgagagacgatcaccactggagcggcgcggcggttgggcgcggaggtcggcagcaaaaacaagcgactcgccgagcaaacccgaatcagccttcagacggtcgtgcctaacaacacgccgttctaccccgccttcttcgcgccccttcgcgtccaagcatccttcaagtttatctctctagttcaacttcaagaagaacaacaccaccaacaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgaattcccggggtac

SEQ ID NO: 6 - DNA, 1314 bp, Piromyces sp.
  E2 xylose isomerase protein “XylA,” codon optimized
atggctaaggagtacttcccccagatccagaagattaagttcgagggtaaggacagcaagaacccgctcgcctttcattactacgacgccgagaaggaggtgatgggcaagaagatgaaggactggcttcgctttgctatggcttggtggcacactctctgcgctgagggcgcggaccagtttggcggcggtacgaagagctttccgtggaacgagggcactgacgctattgagattgctaagcagaaggttgacgctggtttcgagattatgcagaagctcggtattccgtactactgctttcacgatgtcgacctcgtttccgagggcaactcgatcgaggagtacgagtcgaacctcaaggctgtggttgcctacctcaaggagaagcagaaggagaccggaatcaagctcctctggagcaccgccaacgttttcggccacaagcgctacatgaacggcgcctccaccaaccctgacttcgatgttgttgcccgcgctattgtccagattaagaacgccatcgacgctggtatcgagctcggagccgagaactacgttttttggggcggacgcgagggttacatgtccctcctcaacaccgaccagaagcgtgagaaggagcacatggccactatgcttaccatggcccgcgactacgcccgcagcaagggttttaagggtacttttctcattgagccgaagcccatggagccgaccaagcaccagtacgacgtcgacaccgagaccgccattggcttccttaaggcccacaaccttgacaaggattttaaggtgaacatcgaggttaaccacgctacgcttgccggccacacctttgagcatgagctcgcctgcgctgttgacgccggaatgcttggttccattgacgccaaccgcggcgactaccagaacggctgggacaccgaccagtttccgattgaccagtacgagctcgtccaggcctggatggagatcatccgtggtggaggctttgttaccggtggtacgaacttcgacgccaagacgcgccgtaacagcacggacctcgaggacatcatcattgctcatgtgtcgggcatggacgccatggctcgcgcccttgagaacgctgctaagctcctccaggagagcccctacacgaagatgaagaaggagcgctacgcgtcgtttgacagcggaatcggtaaggacttcgaggatggcaagctcaccctggagcaggtgtacgagtacggtaagaagaacggcgagccgaagcagaccagcggcaagcaggagctctacgaggccattgtcgccatgtaccagtag

SEQ ID NO: 7 - DNA, 1485 bp, Piromyces sp.
  E2 xylulose kinase protein “XylB,” codon optimized
atgaagaccgtcgccggcatcgatcttggaacccagtccatgaaggttgtcatttacgactacgagaagaaggagatcatcgagtccgcctcgtgccctatggagctcattagcgagtcggacggaacccgcgagcagacgactgagtggtttgacaagggtctcgaggtgtgctttggaaagctctccgctgataacaagaagaccattgaggcgattggcatctccggccagctccacggcttcgtccctctcgatgcgaacggaaaggcgctctacaacatcaagctctggtgcgacaccgccactgtggaggagtgcaagatcattactgacgccgccggcggcgacaaggctgtcatcgacgcgctcggcaacctcatgctcaccggattcaccgccccgaagattctctggctcaagcgcaacaagcccgaggcctttgctaacctcaagtacattatgctgccccacgattacctcaactggaagctgactggagactacgtcatggagtacggcgacgcctccggcaccgccctttttgattcgaagaaccgctgctggtcgaagaagatttgcgacattattgatcctaagctgctcgaccttctccctaagctcattgagccctcggcccccgccggtaaggtcaacgacgaggccgccaaggcgtacggcattcccgccggaatccccgtttccgctggcggcggtgataacatgatgggtgcggtcggtactggcaccgtcgctgacggattcctcacgatgagcatgggcacctccggaactctttacggctactcggacaagcctatttccgacccggctaacggcctcagcggcttctgcagctccacgggcggctggcttcccctcctttgcaccatgaactgcaccgtcgccaccgagttcgtccgcaacctttttcagatggatatcaaggagctgaacgtcgaggctgctaagtccccctgcggcagcgagggcgttcttgtcattcctttcttcaacggcgagcgcaccccgaacctccccaacggccgcgcctcgattaccggcctcacctccgcgaacacgtcccgcgccaacatcgctcgcgcctcctttgagtcggccgtctttgccatgcgcggtggcctcgatgcgtttcgtaagctcggattccagcccaaggagattcgcctcatcggcggtggttcgaagtccgacctctggcgccagatcgctgctgacattatgaaccttcccatccgtgtcccccttctcgaggaggccgccgccctcggcggagctgtccaggccctttggtgccttaagaaccagtccggtaagtgcgacatcgtcgagctttgcaaggagcatatcaagattgacgagtccaagaacgccaacccgattgccgagaacgtcgccgtgtacgataaggcctacgatgagtactgcaaggtcgttaacacgctcagccctctgtacgcctaa

SEQ ID NO: 8 - DNA, 19 bp, Artificial Sequence
  Primer 5′ CL0130
cctcgggcggcgtcctctt

SEQ ID NO: 9 - DNA, 20 bp, Artificial Sequence
  Primer 3′ CL0130
ggcggccttctcctggttgc

SEQ ID NO: 10 - DNA, 24 bp, Artificial Sequence
  Primer 5′ CL0131
ctactccgttgttgccgccatcct

SEQ ID NO: 11 - DNA, 22 bp, Primer 3′ CL0131
ccgccgaccataccgagaacga

SEQ ID NO: 12 - DNA, 9291 bp, Artificial Sequence
  vector pCL0120
ctcttatctgcctcgcgccgttgaccgccgcttgactcttggcgcttgccgctcgcatcctgcctcgctcgcgcaggcgggcgggcgagtgggtgggtccgcagccttccgcgctcgcccgctagctcgctcgcgccgtgctgcagccagcagggcagcaccgcacggcaggcaggtcccggcgcggatcgatcgatccatcgatccatcgatccatcgatcgtgcggtcaaaaagaaaggaagaagaaaggaaaaagaaaggcgtgcgcacccgagtgcgcgctgagcgcccgctcgcggtcccgcggagcctccgcgttagtccccgccccgcgccgcgcagtcccccgggaggcatcgcgcacctctcgccgccccctcgcgcctcgccgattccccgcctccccttttccgcttcttcgccgcctccgctcgcggccgcgtcgcccgcgccccgctccctatctgctccccaggggggcactccgcaccttttgcgcccgctgccgccgccgcggccgccccgccgccctggtttcccccgcgagcgcggccgcgtcgccgcgcaaagactcgccgcgtgccgccccgagcaacgggtggcggcggcgcggcggcgggcggggcgcggcggcgcgtaggcggggctaggcgccggctaggcgaaacgccgcccccgggcgccgccgccgcccgctccagagcagtcgccgcgccagaccgccaacgcagagaccgagaccgaggtacgtcgcgcccgagcacgccgcgacgcgcggcagggacgaggagcacgacgccgcgccgcgccgcgcggggggggggagggagaggcaggacgcgggagcgagcgtgcatgtttccgcgcgagacgacgccgcgcgcgctggagaggagataaggcgcttggatcgcgagagggccagccaggctggaggcgaaaatgggtggagaggatagtatcttgcgtgcttggacgaggagactgacgaggaggacggatacgtcgatgatgatgtgcacagagaagaagcagttcgaaagcgactactagcaagcaagggatccatgaagttcgcgacctcggtcgcaattttgcttgtggccaacatagccaccgccctcgcgcagagcgatggctgcacccccaccgaccagacgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctgggacacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcaccaccatcaccaccactaacatatgagttatgagatccgaaagtgaaccttgtcctaacccgacagcgaatggcgggagggggcgggctaaaagatcgtattacatagtatttttcccctactctttgtgtttgtcttttttttttttttgaacgcattcaagccacttgtctgggtttacttgtttgtttgcttgcttgcttgcttgcttgcctgcttcttggtcagacggcccaaaaaagggaaaaaattcattcatggcacagataagaaaaagaaaaagtttgtcgaccaccgtcatcagaaagcaagagaagagaaacactcgcgctcacattctcgctcgcgtaagaatcttagccacgcatacgaagtaatttgtccatctggcgaatctttacatgagcgttttcaagctggagcgtgagatcatacctttcttgatcgtaatgttccaaccttgcataggcctcgttgcgatccgctagcaatgcgtcgtactcccgttgcaactgcgccatcgcctcattgtgacgtgagttcagattcttctcgagaccttcgagcgctgctaatttcgcctgacgctccttcttttgtgcttccatgacacgccgcttcaccgtgcgttccacttcttcctcagacatgcccttggctgcctcgacctgctcggtaaaacgggccccagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatacatggtcgacctgcaggaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaagctttgcctcaacgcaactaggcccaggcctactttcactgtgtcttgtcttgcctttcacaccgaccgagtgtgcacaaccgtgttttgcacaaagcgcaagatgctcactcgactgtgaagcaaaggttgcgcgcaagcgactgcgactgcgaggatgaggatgactggcagcctgttcaaaaactgaaaatccgcgatgggtcagctgccattcgcgcatgacgcctgcgagagacaagttaactcgtgtcactggcatgtcctagcatctttacgcgagcaaaattcaatcgctttattttttcagtttcgtaaccttctcgcaaccgcgaatcgccgtttcagcctgactaatctgcagctgcgtggcactgtcagtcagtcagtcagtcgtgcgcgctgttccagcaccgaggtcgcgcgtcgccgcgcctggaccgctgctgctactgctagtggcacggcaggtaggagcttgttgccggaacaccagcagccgccagtcgacgccagccaggggaaagtccggcgtcgaagggagaggaaggcggcgtgtgcaaactaacgttgaccactggcgcccgccgacacgagcaggaagcaggcagctgcagagcgcagcgcgcaagtgcagaatgcgcgaaagatccacttgcgcgcggcgggcgcgcacttgcgggcgcggcgcggaacagtgcggaaaggagcggtgcagacggcgcgcagtgacagtgggcgcaaagccgcgcagtaagcagcggcggggaacggtatacgcagtgccgcgggccgccgcacacagaagtatacgcgggccgaagtggggcgtcgcgcgcgggaagtgcggaatggcgggcaaggaaaggaggagacggaaagagggcgggaaagagagagagagagagtgaaaaaagaaagaaagaaagaaagaaagaaagaaagctcggagccacgccgcggggagagagagaaatgaaagcacggcacggcaaagcaaagcaaagcagacccagccagacccagccgagggaggagcgcgcgcaggacccgcgcggcgagcgagcgagcacggcgcgcgagcgagcgagcgagcgagcgcgcgagcgagcaaggcttgctgcgagcgatcgagcgagcgagcgggaaggatgagcgcgacccgcgcggcgacgaggacagcggcggcgctgtcctcggcgctgacgacgcctgtaaagcagcagcagcagcagcagctgcgcgtaggcgcggcgtcggcacggctggcggccgcggcgttctcgtccggcacgggcggagacgcggccaagaaggcggccgcggcgagggcgttctccacgggacgcggccccaacgcgacacgcgagaagagctcgctggccacggtccaggcggcgacggacgatgcgcgcttcgtcggcctgaccggcgcccaaatctttcatgagctcatgcgcgagcaccaggtggacaccatctttggctaccctggcggcgccattctgcccgtttttgatgccatttttgagagtgacgcgcttcaagttcattctcgctcgccacgagcagggcgccggccacatggccgagggctacgcgcgcgccacgggcaagcccggcgttgtcctcgtcacctcgggccctggagccaccaacaccatcaccccgatcatggatgcttacatggacggtacgccgctgctcgtgttcaccggccaggtgcagacctctgctgtcggcacggacgctttccaggagtgtgacattgttggcatcagccgcgcgtgcaccaagtggaacgtcatggtcaaggacgtgaaggagctcccgcgccgcatcaatgaggcctttgagattgccatgagcggccgcccgggtcccgtgctcgtcgatcttcctaaggatgtgaccgccgttgagctcaaggaaatgcccgacagctccccccaggttgctgtgcgccagaagcaaaaggtcgagcttttccacaaggagcgcattggcgctcctggcacggccgacttcaagctcattgccgagatgatcaaccgtgcggagcgacccgtcatctatgctggccagggtgtcatgcagagcccgttgaatggcccggctgtgctcaaggagttcgcggagaaggccaacattcccgtgaccaccaccatgcagggtctcggcggctttgacgagcgtagtcccctctccctcaagatgctcggcatgcacggctctgcctacgccaactactcgatgcagaacgccgatcttatcctggcgctcggtgcccgctttgatgatcgtgtgacgggccgcgttgacgcctttgctccggaggctcgccgtgccgagcgcgagggccgcggtggcatcgttcactttgagatttcccccaagaacctccacaaggtcgtccagcccaccgtcgcggtcctcggcgacgtggtcgagaacctcgccaacgtcacgccccacgtgcagcgccaggagcgcgagccgtggtttgcgcagatcgccgattggaaggagaagcacccttttctgctcgagtctgttgattcggacgacaaggttctcaagccgcagcaggtcctcacggagcttaacaagcagattctcgagattcaggagaaggacgccgaccaggaggtctacatcaccacgggcgtcggaagccaccagatgcaggcagcgcagttccttacctggaccaagccgcgccagtggatctcctcgggtggcgccggcactatgggctacggccttccctcggccattggcgccaagattgccaagcccgatgctattgttattgacatcgatggtgatgcttcttattcgatgaccggtatggaattgatcacagcagccgaattcaaggttggcgtgaagattcttcttttgcagaacaactttcagggcatggtcaagaacgttcaggatctcttttacgacaagcgctactcgggccaccgccatgttcaacccgcgcttcgacaaggtcgccgatgcgatgcgtgccaagggtctctactgcgcgaaacagtcggagctcaaggacaagatcaaggagtttctcgagtacgatgagggtcccgtcctcctcgaggttttcgtggacaaggacacgctcgtcttgcccatggtccccgctggctttccgctccacgagatggtcctcgagcctcctaagcccaaggacgcctaagttcttttttccatggcgggcgagcgagcgagcgcgcgagcgcgcaagtgcgcaagcgccttgccttgctttgcttcgcttcgctttgctttgcttcacacaacctaagtatgaattcaagttttcttgcttgtcggcgatgcctgcctgccaaccagccagccatccggccggccgtccttgacgccttcgcttccggcgcggccatcgattcaattcacccatccgatacgttccgccccctcacgtccgtctgcgcacgacccctgcacgaccacgccaaggccaacgcgccgctcagctcagcttgtcgacgagtcgcacgtcacatatctcagatgcatttggactgtgagtgttattatgccactagcacgcaacgatcttcggggtcctcgctcattgcatccgttcgggccctgcaggcgtggacgcgagtcgccgccgagacgctgcagcaggccgctccgacgcgagggctcgagctcgccgcgcccgcgcgatgtctgcctggcgccgactgatctctggagcgcaaggaagacacggcgacgcgaggaggaccgaagagagacgctggggtatgcaggatatacccggggcgggacattcgttccgcatacactcccccattcgagcttgctcgtccttggcagagccgagcgcgaacggttccgaacgcggcaaggattttggctctggtgggtggactccgatcgaggcgcaggttctccgcaggttctcgcaggccggcagtggtcgttagaaatagggagtgccggagtcttgacgcgccttagctcactctccgcccacgcgcgcatcgccgccatgccgccgtcccgtctgtcgctgcgctggccgcgaccggctgcgccagagtacgacagtgggacagagctcgaggcgacgcgaatcgctcgggttgtaagggtttcaagggtcgggcgtcgtcgcgtgccaaagtgaaaatagtaggggggggggggggtac

SEQ ID NO: 13 - DNA, 6219 bp, Artificial Sequence
  vector pCL0123
ctcttatctgcctcgcgccgttgaccgccgcttgactcttggcgcttgccgctcgcatcctgcctcgctcgcgcaggcgggcgggcgagtgggtgggtccgcagccttccgcgctcgcccgctagctcgctcgcgccgtgctgcagccagcagggcagcaccgcacggcaggcaggtcccggcgcggatcgatcgatccatcgatccatcgatccatcgatcgtgcggtcaaaaagaaaggaagaagaaaggaaaaagaaaggcgtgcgcacccgagtgcgcgctgagcgcccgctcgcggtcccgcggagcctccgcgttagtccccgccccgcgccgcgcagtcccccgggaggcatcgcgcacctctcgccgccccctcgcgcctcgccgattccccgcctccccttttccgcttcttcgccgcctccgctcgcggccgcgtcgcccgcgccccgctccctatctgctccccaggggggcactccgcaccttttgcgcccgctgccgccgccgcggccgccccgccgccctggtttcccccgcgagcgcggccgcgtcgccgcgcaaagactcgccgcgtgccgccccgagcaacgggtggcggcggcgcggcggcgggcggggcgcggcggcgcgtaggcggggctaggcgccggctaggcgaaacgccgcccccgggcgccgccgccgcccgctccagagcagtcgccgcgccagaccgccaacgcagagaccgagaccgaggtacgtcgcgcccgagcacgccgcgacgcgcggcagggacgaggagcacgacgccgcgccgcgccgcgcggggggggggagggagaggcaggacgcgggagcgagcgtgcatgtttccgcgcgagacgacgccgcgcgcgctggagaggagataaggcgcttggatcgcgagagggccagccaggctggaggcgaaaatgggtggagaggatagtatcttgcgtgcttggacgaggagactgacgaggaggacggatacgtcgatgatgatgtgcacagagaagaagcagttcgaaagcgactactagcaagcaagggatccatgaagttcgcgacctcggtcgcaattttgcttgtggccaacatagccaccgccctcgcgcagagcgatggctgcacccccaccgaccagacgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctgggacacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcaccaccatcaccaccactaacatatgagttatgagatccgaaagtgaaccttgtcctaacccgacagcgaatggcgggagggggcgggctaaaagatcgtattacatagtatttttcccctactctttgtgtttgtcttttttttttttttgaacgcattcaagccacttgtctgggtttacttgtttgtttgcttgcttgcttgcttgcttgcctgcttcttggtcagacggcccaaaaaagggaaaaaattcattcatggcacagataagaaaaagaaaaagtttgtcgaccaccgtcatcagaaagcaagagaagagaaacactcgcgctcacattctcgctcgcgtaagaatcttagccacgcatacgaagtaatttgtccatctggcgaatctttacatgagcgttttcaagctggagcgtgagatcatacctttcttgatcgtaatgttccaaccttgcataggcctcgttgcgatccgctagcaatgcgtcgtactcccgttgcaactgcgccatcgcctcattgtgacgtgagttcagattcttctcgagaccttcgagcgctgctaatttcgcctgacgctccttcttttgtgcttccatgacacgccgcttcaccgtgcgttccacttcttcctcagacatgcccttggctgcctcgacctgctcggtaaaacgggccccagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatacatggtcgacctgcaggaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaagcttccaattttaggccccccactgaccgaggtctgtcgataatccacttttccattgattttccaggtttcgttaactcatgccactgagcaaaacttcggtctttcctaacaaaagctctcctcacaaagcatggcgcggcaacggacgtgtcctcatactccactgccacacaaggtcgataaactaagctcctcacaaatagaggagaattccactgacaactgaaaacaatgtatgagagacgatcaccactggagcggcgcggcggttgggcgcggaggtcggcagcaaaaacaagcgactcgccgagcaaacccgaatcagccttcagacggtcgtgcctaacaacacgccgttctaccccgccttcttcgcgccccttcgcgtccaagcatccttcaagtttatctctctagttcaacttcaagaagaacaacaccaccaacaccatgatgcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgtggacggtgccgacaggtgcttctcgatctgcatcctgggatcaaagccatagtgaaggacagtgatggacagccgacggcagttgggattcgtgaattgctgccctctggttatgtgtgggagggctaacacgtgctccgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgaattcccggggtac

SEQ ID NO: 14 - DNA, 10029 bp, Artificial Sequence
  vector pCL0130
ctcttatctgcctcgcgccgttgaccgccgcttgactcttggcgcttgccgctcgcatcctgcctcgctcgcgcaggcgggcgggcgagtgggtgggtccgcagccttccgcgctcgcccgctagctcgctcgcgccgtgctgcagccagcagggcagcaccgcacggcaggcaggtcccggcgcggatcgatcgatccatcgatccatcgatccatcgatcgtgcggtcaaaaagaaaggaagaagaaaggaaaaagaaaggcgtgcgcacccgagtgcgcgctgagcgcccgctcgcggtcccgcggagcctccgcgttagtccccgccccgcgccgcgcagtcccccgggaggcatcgcgcacctctcgccgccccctcgcgcctcgccgattccccgcctccccttttccgcttcttcgccgcctccgctcgcggccgcgtcgcccgcgccccgctccctatctgctccccaggggggcactccgcaccttttgcgcccgctgccgccgccgcggccgccccgccgccctggtttcccccgcgagcgcggccgcgtcgccgcgcaaagactcgccgcgtgccgccccgagcaacgggtggcggcggcgcggcggcgggcggggcgcggcggcgcgtaggcggggctaggcgccggctaggcgaaacgccgcccccgggcgccgccgccgcccgctccagagcagtcgccgcgccagaccgccaacgcagagaccgagaccgaggtacgtcgcgcccgagcacgccgcgacgcgcggcagggacgaggagcacgacgccgcgccgcgccgcgcggggggggggagggagaggcaggacgcgggagcgagcgtgcatgtttccgcgcgagacgacgccgcgcgcgctggagaggagataaggcgcttggatcgcgagagggccagccaggctggaggcgaaaatgggtggagaggatagtatcttgcgtgcttggacgaggagactgacgaggaggacggatacgtcgatgatgatgtgcacagagaagaagcagttcgaaagcgactactagcaagcaagggatccatgggcctcgaggataaccgcatggttaagcgctttgtcaacgtgggcgagaagaaggccggtagcaccgccatggccatcattgttggcctcttcgcggcctcgggcggcgtcctcttcggctacgacaccggcactatctcgggcgtcatgactatggactacgttctcgcccgctacccctccaacaagcactccttcaccgctgacgagtcgtcgctcatcgtttccattctttcggtcggcaccttcttcggcgccctctgcgccccgttcctcaacgataccctcggccgccgctggtgcctcatcctcagcgccctcattgtctttaacatcggcgccatcctccaggtcatttccaccgccatccccctgctctgcgcgggccgcgttatcgccggtttcggtgtcggcctcatttccgccaccatcccgctctaccagtccgagactgctccgaagtggattcgcggcgccatcgtttcctgctaccagtgggccatcactatcggacttttcctcgcttcctgcgtcaacaagggcaccgagcacatgaccaactccggttcgtaccgtattcctctggccatccagtgcctctggggcctcatccttggtattggcatgattttcctccctgagaccccccgcttctggatttcgaagggcaaccaggagaaggccgccgagtccctcgcccgtctccgcaagctccccatcgaccatcctgatagccttgaggagcttcgcgatattactgccgcctacgagttcgagaccgtctacggtaagtccagctggtcccaggtcttttcccacaagaaccatcagctcaagcgcctctttaccggcgttgccattcaggcctttcagcagctcaccggagttaactttatcttttactacggcaccaccttttttaagcgcgccggagtcaacggattcaccatcagccttgccaccaacatcgttaacgtcggcagcactattcccggcattcttctcatggaggtcctcggccgccgcaacatgctcatgggcggtgccaccggcatgtcgctgtcgcagcttatcgtcgccattgtcggagttgccacgtcggagaacaacaagtcgagccagtcggtcctcgtcgctttctcgtgcatctttatcgctttttttgccgccacctggggtccctgcgcctgggtcgtcgtcggcgagctctttccccttcgcactcgcgctaagtccgtttccctctgcaccgcgtccaactggctctggaactggggcattgcttacgccaccccctacatggtcgacgaggataagggtaacctcggcagcaacgttttttttatttggggaggcttcaacctcgcttgcgtctttttcgcgtggtacttcatttacgagaccaagggcctttccctcgagcaggttgatgagctctacgagcatgtttcgaaggcgtggaagtccaagggttttgtcccgtccaagcactcctttcgcgagcaggtcgaccagcagatggactccaagaccgaggccattatgagcgaggaggcgtcggtttaacatatgagttatgagatccgaaagtgaaccttgtcctaacccgacagcgaatggcgggagggggcgggctaaaagatcgtattacatagtatttttcccctactctttgtgtttgtcttttttttttttttgaacgcattcaagccacttgtctgggtttacttgtttgtttgcttgcttgcttgcttgcttgcctgcttcttggtcagacggcccaaaaaagggaaaaaattcattcatggcacagataagaaaaagaaaaagtttgtcgaccaccgtcatcagaaagcaagagaagagaaacactcgcgctcacattctcgctcgcgtaagaatcttagccacgcatacgaagtaatttgtccatctggcgaatctttacatgagcgttttcaagctggagcgtgagatcatacctttcttgatcgtaatgttccaaccttgcataggcctcgttgcgatccgctagcaatgcgtcgtactcccgttgcaactgcgccatcgcctcattgtgacgtgagttcagattcttctcgagaccttcgagcgctgctaatttcgcctgacgctccttcttttgtgcttccatgacacgccgcttcaccgtgcgttccacttcttcctcagacatgcccttggctgcctcgacctgctcggtaaaacgggccccagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatacatggtcgacctgcaggaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaagctttgcctcaacgcaactaggcccaggcctactttcactgtgtcttgtcttgcctttcacaccgaccgagtgtgcacaaccgtgttttgcacaaagcgcaagatgctcactcgactgtgaagcaaaggttgcgcgcaagcgactgcgactgcgaggatgaggatgactggcagcctgttcaaaaactgaaaatccgcgatgggtcagctgccattcgcgcatgacgcctgcgagagacaagttaactcgtgtcactggcatgtcctagcatctttacgcgagcaaaattcaatcgctttattttttcagtttcgtaaccttctcgcaaccgcgaatcgccgtttcagcctgactaatctgcagctgcgtggcactgtcagtcagtcagtcagtcgtgcgcgctgttccagcaccgaggtcgcgcgtcgccgcgcctggaccgctgctgctactgctagtggcacggcaggtaggagcttgttgccggaacaccagcagccgccagtcgacgccagccaggggaaagtccggcgtcgaagggagaggaaggcggcgtgtgcaaactaacgttgaccactggcgcccgccgacacgagcaggaagcaggcagctgcagagcgcagcgcgcaagtgcagaatgcgcgaaagatccacttgcgcgcggcgggcgcgcacttgcgggcgcggcgcggaacagtgcggaaaggagcggtgcagacggcgcgcagtgacagtgggcgcaaagccgcgcagtaagcagcggcggggaacggtatacgcagtgccgcgggccgccgcacacagaagtatacgcgggccgaagtggggcgtcgcgcgcgggaagtgcggaatggcgggcaaggaaaggaggagacggaaagagggcgggaaagagagagagagagagtgaaaaaagaaagaaagaaagaaagaaagaaagaaagctcggagccacgccgcggggagagagagaaatgaaagcacggcacggcaaagcaaagcaaagcagacccagccagacccagccgagggaggagcgcgcgcaggacccgcgcggcgagcgagcgagcacggcgcgcgagcgagcgagcgagcgagcgcgcgagcgagcaaggcttgctgcgagcgatcgagcgagcgagcgggaaggatgagcgcgacccgcgcggcgacgaggacagcggcggcgctgtcctcggcgctgacgacgcctgtaaagcagcagcagcagcagcagctgcgcgtaggcgcggcgtcggcacggctggcggccgcggcgttctcgtccggcacgggcggagacgcggccaagaaggcggccgcggcgagggcgttctccacgggacgcggccccaacgcgacacgcgagaagagctcgctggccacggtccaggcggcgacggacgatgcgcgcttcgtcggcctgaccggcgcccaaatctttcatgagctcatgcgcgagcaccaggtggacaccatctttggctaccctggcggcgccattctgcccgtttttgatgccatttttgagagtgacgcgcttcaagttcattctcgctcgccacgagcagggcgccggccacatggccgagggctacgcgcgcgccacgggcaagcccggcgttgtcctcgtcacctcgggccctggagccaccaacaccatcaccccgatcatggatgcttacatggacggtacgccgctgctcgtgttcaccggccaggtgcagacctctgctgtcggcacggacgctttccaggagtgtgacattgttggcatcagccgcgcgtgcaccaagtggaacgtcatggtcaaggacgtgaaggagctcccgcgccgcatcaatgaggcctttgagattgccatgagcggccgcccgggtcccgtgctcgtcgatcttcctaaggatgtgaccgccgttgagctcaaggaaatgcccgacagctccccccaggttgctgtgcgccagaagcaaaaggtcgagcttttccacaaggagcgcattggcgctcctggcacggccgacttcaagctcattgccgagatgatcaaccgtgcggagcgacccgtcatctatgctggccagggtgtcatgcagagcccgttgaatggcccggctgtgctcaaggagttcgcggagaaggccaacattcccgtgaccaccaccatgcagggtctcggcggctttgacgagcgtagtcccctctccctcaagatgctcggcatgcacggctctgcctacgccaactactcgatgcagaacgccgatcttatcctggcgctcggtgcccgctttgatgatcgtgtgacgggccgcgttgacgcctttgctccggaggctcgccgtgccgagcgcgagggccgcggtggcatcgttcactttgagatttcccccaagaacctccacaaggtcgtccagcccaccgtcgcggtcctcggcgacgtggtcgagaacctcgccaacgtcacgccccacgtgcagcgccaggagcgcgagccgtggtttgcgcagatcgccgattggaaggagaagcacccttttctgctcgagtctgttgattcggacgacaaggttctcaagccgcagcaggtcctcacggagcttaacaagcagattctcgagattcaggagaaggacgccgaccaggaggtctacatcaccacgggcgtcggaagccaccagatgcaggcagcgcagttccttacctggaccaagccgcgccagtggatctcctcgggtggcgccggcactatgggctacggccttccctcggccattggcgccaagattgccaagcccgatgctattgttattgacatcgatggtgatgcttcttattcgatgaccggtatggaattgatcacagcagccgaattcaaggttggcgtgaagattcttcttttgcagaacaactttcagggcatggtcaagaacgttcaggatctcttttacgacaagcgctactcgggccaccgccatgttcaacccgcgcttcgacaaggtcgccgatgcgatgcgtgccaagggtctctactgcgcgaaacagtcggagctcaaggacaagatcaaggagtttctcgagtacgatgagggtcccgtcctcctcgaggttttcgtggacaaggacacgctcgtcttgcccatggtccccgctggctttccgctccacgagatggtcctcgagcctcctaagcccaaggacgcctaagttcttttttccatggcgggcgagcgagcgagcgcgcgagcgcgcaagtgcgcaagcgccttgccttgctttgcttcgcttcgctttgctttgcttcacacaacctaagtatgaattcaagttttcttgcttgtcggcgatgcctgcctgccaaccagccagccatccggccggccgtccttgacgccttcgcttccggcgcggccatcgattcaattcacccatccgatacgttccgccccctcacgtccgtctgcgcacgacccctgcacgaccacgccaaggccaacgcgccgctcagctcagcttgtcgacgagtcgcacgtcacatatctcagatgcatttggactgtgagtgttattatgccactagcacgcaacgatcttcggggtcctcgctcattgcatccgttcgggccctgcaggcgtggacgcgagtcgccgccgagacgctgcagcaggccgctccgacgcgagggctcgagctcgccgcgcccgcgcgatgtctgcctggcgccgactgatctctggagcgcaaggaagacacggcgacgcgaggaggaccgaagagagacgctggggtatgcaggatatacccggggcgggacattcgttccgcatacactcccccattcgagcttgctcgtccttggcagagccgagcgcgaacggttccgaacgcggcaaggattttggctctggtgggtggactccgatcgaggcgcaggttctccgcaggttctcgcaggccggcagtggtcgttagaaatagggagtgccggagtcttgacgcgccttagctcactctccgcccacgcgcgcatcgccgccatgccgccgtcccgtctgtcgctgcgctggccgcgaccggctgcgccagagtacgacagtgggacagagctcgaggcgacgcgaatcgctcgggttgtaagggtttcaagggtcgggcgtcgtcgcgtgccaaagtgaaaatagtaggggggggggggggtac

SEQ ID NO: 15 - DNA, 9972 bp, Artificial Sequence
  vector pCL0131
ctcttatctgcctcgcgccgttgaccgccgcttgactcttggcgcttgccgctcgcatcctgcctcgctcgcgcaggcgggcgggcgagtgggtgggtccgcagccttccgcgctcgcccgctagctcgctcgcgccgtgctgcagccagcagggcagcaccgcacggcaggcaggtcccggcgcggatcgatcgatccatcgatccatcgatccatcgatcgtgcggtcaaaaagaaaggaagaagaaaggaaaaagaaaggcgtgcgcacccgagtgcgcgctgagcgcccgctcgcggtcccgcggagcctccgcgttagtccccgccccgcgccgcgcagtcccccgggaggcatcgcgcacctctcgccgccccctcgcgcctcgccgattccccgcctccccttttccgcttcttcgccgcctccgctcgcggccgcgtcgcccgcgccccgctccctatctgctccccaggggggcactccgcaccttttgcgcccgctgccgccgccgcggccgccccgccgccctggtttcccccgcgagcgcggccgcgtcgccgcgcaaagactcgccgcgtgccgccccgagcaacgggtggcggcggcgcggcggcgggcggggcgcggcggcgcgtaggcggggctaggcgccggctaggcgaaacgccgcccccgggcgccgccgccgcccgctccagagcagtcgccgcgccagaccgccaacgcagagaccgagaccgaggtacgtcgcgcccgagcacgccgcgacgcgcggcagggacgaggagcacgacgccgcgccgcgccgcgcggggggggggagggagaggcaggacgcgggagcgagcgtgcatgtttccgcgcgagacgacgccgcgcgcgctggagaggagataaggcgcttggatcgcgagagggccagccaggctggaggcgaaaatgggtggagaggatagtatcttgcgtgcttggacgaggagactgacgaggaggacggatacgtcgatgatgatgtgcacagagaagaagcagttcgaaagcgactactagcaagcaagggatccatggccctcgaccctgagcagcagcagcccatttcctccgtgtcgcgcgagtttggtaagtcgtccggtgagatctcccccgagcgtgagcctctcattaaggagaaccacgtccccgagaactactccgttgttgccgccatcctccccttcctcttcccggccctgggtggcctcctttacggttacgagattggcgctacgtcgtgcgctacgatttcccttcagtccccctccctctccggcatctcctggtacaacctctcctccgtcgatgttggcctcgtcacttccggttccctctacggtgctctgtttggctccattgttgccttcaccattgccgacgttattggccgtcgcaaggagcttatcctcgctgctctcctctacctcgtcggtgccctcgttaccgctctcgcccctacgtactccgttctcatcatcggccgtgtcatttacggtgtttccgtcggtcttgccatgcatgctgcccctatgtacatcgcggagaccgccccgtcccccatccgcggccagctcgtttccctcaaggagtttttcatcgttctcggtatggtcggcggatacggcattggttccctcaccgtcaacgtccactccggttggcgctacatgtacgctacctccgttcccctcgctgtgatcatgggcattggcatgtggtggcttcctgcctccccccgttggctcctcctccgcgtcattcagggtaagggtaacgttgagaaccagcgcgaggctgccattaagtccctctgctgcctccgtggtcctgccttcgtcgactcggccgccgagcaggtcaacgagattctcgccgagcttaccttcgttggcgaggataaggaggtcaccttcggcgagctcttccagggaaagtgcctcaaggccctcattatcggcggcggccttgttctctttcagcagatcaccggtcagccttcggtcctctactacgccccctcgatcctccagactgcgggcttctccgccgccggcgatgctacccgcgtttccattcttctcggcctcctcaagctcattatgaccggtgtcgccgtcgtcgttatcgatcgtctcggccgtcgccctctcctcctcggcggagtcggtggtatggttgtttcgctctttctccttggctcgtactaccttttcttcagcgcttcccccgtcgtcgccgttgtcgccctccttctctacgtgggttgctaccagctctcctttggccccattggctggcttatgatttccgagatttttcccctcaagctccgtggtcgcggactctcccttgccgtgcttgtcaactttggtgccaacgccctcgtcacctttgccttttcccctctcaaggagctcctcggcgccggcatcctgttttgcggctttggcgttatctgcgttctctcccttgtttttatcttttttatcgtcccggagactaagggcctcacgctcgaggagatcgaggcgaagtgcctctaacatatgagttatgagatccgaaagtgaaccttgtcctaacccgacagcgaatggcgggagggggcgggctaaaagatcgtattacatagtatttttcccctactctttgtgtttgtcttttttttttttttgaacgcattcaagccacttgtctgggtttacttgtttgtttgcttgcttgcttgcttgcttgcctgcttcttggtcagacggcccaaaaaagggaaaaaattcattcatggcacagataagaaaaagaaaaagtttgtcgaccaccgtcatcagaaagcaagagaagagaaacactcgcgctcacattctcgctcgcgtaagaatcttagccacgcatacgaagtaatttgtccatctggcgaatctttacatgagcgttttcaagctggagcgtgagatcatacctttcttgatcgtaatgttccaaccttgcataggcctcgttgcgatccgctagcaatgcgtcgtactcccgttgcaactgcgccatcgcctcattgtgacgtgagttcagattcttctcgagaccttcgagcgctgctaatttcgcctgacgctccttcttttgtgcttccatgacacgccgcttcaccgtgcgttccacttcttcctcagacatgcccttggctgcctcgacctgctcggtaaaacgggccccagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatacatggtcgacctgcaggaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaagctttgcctcaacgcaactaggcccaggcctactttcactgtgtcttgtcttgcctttcacaccgaccgagtgtgcacaaccgtgttttgcacaaagcgcaagatgctcactcgactgtgaagcaaaggttgcgcgcaagcgactgcgactgcgaggatgaggatgactggcagcctgttcaaaaactgaaaatccgcgatgggtcagctgccattcgcgcatgacgcctgcgagagacaagttaactcgtgtcactggcatgtcctagcatctttacgcgagcaaaattcaatcgctttattttttcagtttcgtaaccttctcgcaaccgcgaatcgccgtttcagcctgactaatctgcagctgcgtggcactgtcagtcagtcagtcagtcgtgcgcgctgttccagcaccgaggtcgcgcgtcgccgcgcctggaccgctgctgctactgctagtggcacggcaggtaggagcttgttgccggaacaccagcagccgccagtcgacgccagccaggggaaagtccggcgtcgaagggagaggaaggcggcgtgtgcaaactaacgttgaccactggcgcccgccgacacgagcaggaagcaggcagctgcagagcgcagcgcgcaagtgcagaatgcgcgaaagatccacttgcgcgcggcgggcgcgcacttgcgggcgcggcgcggaacagtgcggaaaggagcggtgcagacggcgcgcagtgacagtgggcgcaaagccgcgcagtaagcagcggcggggaacggtatacgcagtgccgcgggccgccgcacacagaagtatacgcgggccgaagtggggcgtcgcgcgcgggaagtgcggaatggcgggcaaggaaaggaggagacggaaagagggcgggaaagagagagagagagagtgaaaaaagaaagaaagaaagaaagaaagaaagaaagctcggagccacgccgcggggagagagagaaatgaaagcacggcacggcaaagcaaagcaaagcagacccagccagacccagccgagggaggagcgcgcgcaggacccgcgcggcgagcgagcgagcacggcgcgcgagcgagcgagcgagcgagcgcgcgagcgagcaaggcttgctgcgagcgatcgagcgagcgagcgggaaggatgagcgcgacccgcgcggcgacgaggacagcggcggcgctgtcctcggcgctgacgacgcctgtaaagcagcagcagcagcagcagctgcgcgtaggcgcggcgtcggcacggctggcggccgcggcgttctcgtccggcacgggcggagacgcggccaagaaggcggccgcggcgagggcgttctccacgggacgcggccccaacgcgacacgcgagaagagctcgctggccacggtccaggcggcgacggacgatgcgcgcttcgtcggcctgaccggcgcccaaatctttcatgagctcatgcgcgagcaccaggtggacaccatctttggctaccctggcggcgccattctgcccgtttttgatgccatttttgagagtgacgcgcttcaagttcattctcgctcgccacgagcagggcgccggccacatggccgagggctacgcgcgcgccacgggcaagcccggcgttgtcctcgtcacctcgggccctggagccaccaacaccatcaccccgatcatggatgcttacatggacggtacgccgctgctcgtgttcaccggccaggtgcagacctctgctgtcggcacggacgctttccaggagtgtgacattgttggcatcagccgcgcgtgcaccaagtggaacgtcatggtcaaggacgtgaaggagctcccgcgccgcatcaatgaggcctttgagattgccatgagcggccgcccgggtcccgtgctcgtcgatcttcctaaggatgtgaccgccgttgagctcaaggaaatgcccgacagctccccccaggttgctgtgcgccagaagcaaaaggtcgagcttttccacaaggagcgcattggcgctcctggcacggccgacttcaagctcattgccgagatgatcaaccgtgcggagcgacccgtcatctatgctggccagggtgtcatgcagagcccgttgaatggcccggctgtgctcaaggagttcgcggagaaggccaacattcccgtgaccaccaccatgcagggtctcggcggctttgacgagcgtagtcccctctccctcaagatgctcggcatgcacggctctgcctacgccaactactcgatgcagaacgccgatcttatcctggcgctcggtgcccgctttgatgatcgtgtgacgggccgcgttgacgcctttgctccggaggctcgccgtgccgagcgcgagggccgcggtggcatcgttcactttgagatttcccccaagaacctccacaaggtcgtccagcccaccgtcgcggtcctcggcgacgtggtcgagaacctcgccaacgtcacgccccacgtgcagcgccaggagcgcgagccgtggtttgcgcagatcgccgattggaaggagaagcacccttttctgctcgagtctgttgattcggacgacaaggttctcaagccgcagcaggtcctcacggagcttaacaagcagattctcgagattcaggagaaggacgccgaccaggaggtctacatcaccacgggcgtcggaagccaccagatgcaggcagcgcagttccttacctggaccaagccgcgccagtggatctcctcgggtggcgccggcactatgggctacggccttccctcggccattggcgccaagattgccaagcccgatgctattgttattgacatcgatggtgatgcttcttattcgatgaccggtatggaattgatcacagcagccgaattcaaggttggcgtgaagattcttcttttgcagaacaactttcagggcatggtcaagaacgttcaggatctcttttacgacaagcgctactcgggccaccgccatgttcaacccgcgcttcgacaaggtcgccgatgcgatgcgtgccaagggtctctactgcgcgaaacagtcggagctcaaggacaagatcaaggagtttctcgagtacgatgagggtcccgtcctcctcgaggttttcgtggacaaggacacgctcgtcttgcccatggtccccgctggctttccgctccacgagatggtcctcgagcctcctaagcccaaggacgcctaagttcttttttccatggcgggcgagcgagcgagcgcgcgagcgcgcaagtgcgcaagcgccttgccttgctttgcttcgcttcgctttgctttgcttcacacaacctaagtatgaattcaagttttcttgcttgtcggcgatgcctgcctgccaaccagccagccatccggccggccgtccttgacgccttcgcttccggcgcggccatcgattcaattcacccatccgatacgttccgccccctcacgtccgtctgcgcacgacccctgcacgaccacgccaaggccaacgcgccgctcagctcagcttgtcgacgagtcgcacgtcacatatctcagatgcatttggactgtgagtgttattatgccactagcacgcaacgatcttcggggtcctcgctcattgcatccgttcgggccctgcaggcgtggacgcgagtcgccgccgagacgctgcagcaggccgctccgacgcgagggctcgagctcgccgcgcccgcgcgatgtctgcctggcgccgactgatctctggagcgcaaggaagacacggcgacgcgaggaggaccgaagagagacgctggggtatgcaggatatacccggggcgggacattcgttccgcatacactcccccattcgagcttgctcgtccttggcagagccgagcgcgaacggttccgaacgcggcaaggattttggctctggtgggtggactccgatcgaggcgcaggttctccgcaggttctcgcaggccggcagtggtcgttagaaatagggagtgccggagtcttgacgcgccttagctcactctccgcccacgcgcgcatcgccgccatgccgccgtcccgtctgtcgctgcgctggccgcgaccggctgcgccagagtacgacagtgggacagagctcgaggcgacgcgaatcgctcgggttgtaagggtttcaagggtcgggcgtcgtcgcgtgccaaagtgaaaatagtaggggggggggggggtac

SEQ ID NO: 16 - DNA, 6634 bp, Artificial Sequence
  vector pCL0132
ctcttatctgcctcgcgccgttgaccgccgcttgactcttggcgcttgccgctcgcatcctgcctcgctcgcgcaggcgggcgggcgagtgggtgggtccgcagccttccgcgctcgcccgctagctcgctcgcgccgtgctgcagccagcagggcagcaccgcacggcaggcaggtcccggcgcggatcgatcgatccatcgatccatcgatccatcgatcgtgcggtcaaaaagaaaggaagaagaaaggaaaaagaaaggcgtgcgcacccgagtgcgcgctgagcgcccgctcgcggtcccgcggagcctccgcgttagtccccgccccgcgccgcgcagtcccccgggaggcatcgcgcacctctcgccgccccctcgcgcctcgccgattccccgcctccccttttccgcttcttcgccgcctccgctcgcggccgcgtcgcccgcgccccgctccctatctgctccccaggggggcactccgcaccttttgcgcccgctgccgccgccgcggccgccccgccgccctggtttcccccgcgagcgcggccgcgtcgccgcgcaaagactcgccgcgtgccgccccgagcaacgggtggcggcggcgcggcggcgggcggggcgcggcggcgcgtaggcggggctaggcgccggctaggcgaaacgccgcccccgggcgccgccgccgcccgctccagagcagtcgccgcgccagaccgccaacgcagagaccgagaccgaggtacgtcgcgcccgagcacgccgcgacgcgcggcagggacgaggagcacgacgccgcgccgcgccgcgcggggggggggagggagaggcaggacgcgggagcgagcgtgcatgtttccgcgcgagacgacgccgcgcgcgctggagaggagataaggcgcttggatcgcgagagggccagccaggctggaggcgaaaatgggtggagaggatagtatcttgcgtgcttggacgaggagactgacgaggaggacggatacgtcgatgatgatgtgcacagagaagaagcagttcgaaagcgactactagcaagcaagggatccatggctaaggagtacttcccccagatccagaagattaagttcgagggtaaggacagcaagaacccgctcgcctttcattactacgacgccgagaaggaggtgatgggcaagaagatgaaggactggcttcgctttgctatggcttggtggcacactctctgcgctgagggcgcggaccagtttggcggcggtacgaagagctttccgtggaacgagggcactgacgctattgagattgctaagcagaaggttgacgctggtttcgagattatgcagaagctcggtattccgtactactgctttcacgatgtcgacctcgtttccgagggcaactcgatcgaggagtacgagtcgaacctcaaggctgtggttgcctacctcaaggagaagcagaaggagaccggaatcaagctcctctggagcaccgccaacgttttcggccacaagcgctacatgaacggcgcctccaccaaccctgacttcgatgttgttgcccgcgctattgtccagattaagaacgccatcgacgctggtatcgagctcggagccgagaactacgttttttggggcggacgcgagggttacatgtccctcctcaacaccgaccagaagcgtgagaaggagcacatggccactatgcttaccatggcccgcgactacgcccgcagcaagggttttaagggtacttttctcattgagccgaagcccatggagccgaccaagcaccagtacgacgtcgacaccgagaccgccattggcttccttaaggcccacaaccttgacaaggattttaaggtgaacatcgaggttaaccacgctacgcttgccggccacacctttgagcatgagctcgcctgcgctgttgacgccggaatgcttggttccattgacgccaaccgcggcgactaccagaacggctgggacaccgaccagtttccgattgaccagtacgagctcgtccaggcctggatggagatcatccgtggtggaggctttgttaccggtggtacgaacttcgacgccaagacgcgccgtaacagcacggacctcgaggacatcatcattgctcatgtgtcgggcatggacgccatggctcgcgcccttgagaacgctgctaagctcctccaggagagcccctacacgaagatgaagaaggagcgctacgcgtcgtttgacagcggaatcggtaaggacttcgaggatggcaagctcaccctggagcaggtgtacgagtacggtaagaagaacggcgagccgaagcagaccagcggcaagcaggagctctacgaggccattgtcgccatgtaccagtagcatatgagttatgagatccgaaagtgaaccttgtcctaacccgacagcgaatggcgggagggggcgggctaaaagatcgtattacatagtatttttcccctactctttgtgtttgtcttttttttttttttgaacgcattcaagccacttgtctgggtttacttgtttgtttgcttgcttgcttgcttgcttgcctgcttcttggtcagacggcccaaaaaagggaaaaaattcattcatggcacagataagaaaaagaaaaagtttgtcgaccaccgtcatcagaaagcaagagaagagaaacactcgcgctcacattctcgctcgcgtaagaatcttagccacgcatacgaagtaatttgtccatctggcgaatctttacatgagcgttttcaagctggagcgtgagatcatacctttcttgatcgtaatgttccaaccttgcataggcctcgttgcgatccgctagcaatgcgtcgtactcccgttgcaactgcgccatcgcctcattgtgacgtgagttcagattcttctcgagaccttcgagcgctgctaatttcgcctgacgctccttcttttgtgcttccatgacacgccgcttcaccgtgcgttccacttcttcctcagacatgcccttggctgcctcgacctgctcggtaaaacgggccccagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatacatggtcgacctgcaggaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaagcttgaggtctgtcgataatccacttttccattgattttccaggtttcgttaactcatgccactgagcaaaacttcggtctttcctaacaaaagctctcctcacaaagcatggcgcggcaacggacgtgtcctcatactccactgccacacaaggtcgataaactaagctcctcacaaatagaggagaattccactgacaactgaaaacaatgtatgagagacgatcaccactggagcggcgcggcggttgggcgcggaggtcggcagcaaaaacaagcgactcgccgagcaaacccgaatcagccttcagacggtcgtgcctaacaacacgccgttctaccccgccttcttcgcgccccttcgcgtccaagcatccttcaagtttatctctctagttcaacttcaagaagaacaacaccaccaacaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcggtac

SEQ ID NO: 17 - DNA, 6277 bp, Artificial Sequence
  vector pCL0133
ctcttatctgcctcgcgccgttgaccgccgcttgactcttggcgcttgccgctcgcatcctgcctcgctcgcgcaggcgggcgggcgagtgggtgggtccgcagccttccgcgctcgcccgctagctcgctcgcgccgtgctgcagccagcagggcagcaccgcacggcaggcaggtcccggcgcggatcgatcgatccatcgatccatcgatccatcgatcgtgcggtcaaaaagaaaggaagaagaaaggaaaaagaaaggcgtgcgcacccgagtgcgcgctgagcgcccgctcgcggtcccgcggagcctccgcgttagtccccgccccgcgccgcgcagtcccccgggaggcatcgcgcacctctcgccgccccctcgcgcctcgccgattccccgcctccccttttccgcttcttcgccgcctccgctcgcggccgcgtcgcccgcgccccgctccctatctgctccccaggggggcactccgcaccttttgcgcccgctgccgccgccgcggccgccccgccgccctggtttcccccgcgagcgcggccgcgtcgccgcgcaaagactcgccgcgtgccgccccgagcaacgggtggcggcggcgcggcggcgggcggggcgcggcggcgcgtaggcggggctaggcgccggctaggcgaaacgccgcccccgggcgccgccgccgcccgctccagagcagtcgccgcgccagaccgccaacgcagagaccgagaccgaggtacgtcgcgcccgagcacgccgcgacgcgcggcagggacgaggagcacgacgccgcgccgcgccgcgcggggggggggagggagaggcaggacgcgggagcgagcgtgcatgtttccgcgcgagacgacgccgcgcgcgctggagaggagataaggcgcttggatcgcgagagggccagccaggctggaggcgaaaatgggtggagaggatagtatcttgcgtgcttggacgaggagactgacgaggaggacggatacgtcgatgatgatgtgcacagagaagaagcagttcgaaagcgactactagcaagcaagggatccatgccctccattaagctcaactccggttacgatatgcccgccgtcggttttggttgctggaaggtggacgtcgacacttgctcggagcagatttaccgcgccattaagaccggataccgcctctttgacggtgccgaggactacgccaacgagaagctggtcggagccggcgtcaagaaggccattgatgagggaattgtcaagcgcgaggacctctttctcacctccaagctctggaacaactaccaccaccccgataacgtcgagaaggctcttaaccgtaccctcagcgatctccaggtcgactacgtcgatctttttcttattcacttccctgtcacgttcaagtttgtccctcttgaggagaagtacccccccggattctactgcggaaagggcgataactttgactacgaggacgttcctattctggagacttggaaggctctcgagaagctcgtcaaggccggcaagattcgcagcatcggcgtcagcaactttcctggagctctcctcctggacctccttcgcggagccaccatcaagccttcggttcttcaggtcgagcaccatccttaccttcagcagccccgtctcatcgagtttgcccagtcccgcggtattgccgtcacggcctacagctccttcggccctcagtcctttgtcgagctcaaccagggtcgcgcccttaacaccagccccctcttcgagaacgagaccattaaggccatcgctgctaagcacggtaagtcccccgcccaggtcctcctccgttggagctcgcagcgcggaatcgccatcatccctaagagcaacaccgtccctcgccttcttgagaacaaggatgtcaactccttcgacctcgatgagcaggatttcgccgacattgccaagctcgatattaacctccgcttcaacgacccctgggactgggataagatccctatctttgtctaacatatgagttatgagatccgaaagtgaaccttgtcctaacccgacagcgaatggcgggagggggcgggctaaaagatcgtattacatagtatttttcccctactctttgtgtttgtcttttttttttttttgaacgcattcaagccacttgtctgggtttacttgtttgtttgcttgcttgcttgcttgcttgcctgcttcttggtcagacggcccaaaaaagggaaaaaattcattcatggcacagataagaaaaagaaaaagtttgtcgaccaccgtcatcagaaagcaagagaagagaaacactcgcgctcacattctcgctcgcgtaagaatcttagccacgcatacgaagtaatttgtccatctggcgaatctttacatgagcgttttcaagctggagcgtgagatcatacctttcttgatcgtaatgttccaaccttgcataggcctcgttgcgatccgctagcaatgcgtcgtactcccgttgcaactgcgccatcgcctcattgtgacgtgagttcagattcttctcgagaccttcgagcgctgctaatttcgcctgacgctccttcttttgtgcttccatgacacgccgcttcaccgtgcgttccacttcttcctcagacatgcccttggctgcctcgacctgctcggtaaaacgggccccagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatacatggtcgacctgcaggaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaagcttgaggtctgtcgataatccacttttccattgattttccaggtttcgttaactcatgccactgagcaaaacttcggtctttcctaacaaaagctctcctcacaaagcatggcgcggcaacggacgtgtcctcatactccactgccacacaaggtcgataaactaagctcctcacaaatagaggagaattccactgacaactgaaaacaatgtatgagagacgatcaccactggagcggcgcggcggttgggcgcggaggtcggcagcaaaaacaagcgactcgccgagcaaacccgaatcagccttcagacggtcgtgcctaacaacacgccgttctaccccgccttcttcgcgccccttcgcgtccaagcatccttcaagtttatctctctagttcaacttcaagaagaacaacaccaccaacaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcggtac

SEQ ID NO: 18 - DNA, 6456 bp, Artificial Sequence
  vector pCL0134
ctcttatctgcctcgcgccgttgaccgccgcttgactcttggcgcttgccgctcgcatcctgcctcgctcgcgcaggcgggcgggcgagtgggtgggtccgcagccttccgcgctcgcccgctagctcgctcgcgccgtgctgcagccagcagggcagcaccgcacggcaggcaggtcccggcgcggatcgatcgatccatcgatccatcgatccatcgatcgtgcggtcaaaaagaaaggaagaagaaaggaaaaagaaaggcgtgcgcacccgagtgcgcgctgagcgcccgctcgcggtcccgcggagcctccgcgttagtccccgccccgcgccgcgcagtcccccgggaggcatcgcgcacctctcgccgccccctcgcgcctcgccgattccccgcctccccttttccgcttcttcgccgcctccgctcgcggccgcgtcgcccgcgccccgctccctatctgctccccaggggggcactccgcaccttttgcgcccgctgccgccgccgcggccgccccgccgccctggtttcccccgcgagcgcggccgcgtcgccgcgcaaagactcgccgcgtgccgccccgagcaacgggtggcggcggcgcggcggcgggcggggcgcggcggcgcgtaggcggggctaggcgccggctaggcgaaacgccgcccccgggcgccgccgccgcccgctccagagcagtcgccgcgccagaccgccaacgcagagaccgagaccgaggtacgtcgcgcccgagcacgccgcgacgcgcggcagggacgaggagcacgacgccgcgccgcgccgcgcggggggggggagggagaggcaggacgcgggagcgagcgtgcatgtttccgcgcgagacgacgccgcgcgcgctggagaggagataaggcgcttggatcgcgagagggccagccaggctggaggcgaaaatgggtggagaggatagtatcttgcgtgcttggacgaggagactgacgaggaggacggatacgtcgatgatgatgtgcacagagaagaagcagttcgaaagcgactactagcaagcaagggatccatgaccgccaacccgagcctcgtccttaacaagatcgacgatatttccttcgagacctacgacgcccccgagatcagcgagcccaccgatgtcctcgtccaggttaagaagaccggcatctgcggttccgatattcacttttacgctcacggacgcattggaaactttgtcctcactaagcctatggttctgggtcacgagtccgccggtactgtcgttcaggtgggaaagggtgttacgtcgcttaaggtcggagacaacgttgccatcgagcccggcatccccagccgcttttccgatgagtacaagtccggtcactacaacctctgcccccacatggctttcgccgccacccccaactccaaggagggcgagcctaacccccccggcaccctctgcaagtactttaagtcccccgaggattttctcgtcaagctccccgaccacgtctcgcttgagctgggcgccctcgtcgagcccctgtccgtcggagttcacgccagcaagctcggtagcgttgcctttggcgactacgtggccgtttttggcgcgggtcctgtcggccttctcgccgccgctgtggccaagacctttggagccaagggcgttatcgtcgtcgacatttttgacaacaagctcaagatggctaaggatattggcgccgctactcatacctttaactccaagaccggcggttccgaggagcttatcaaggcctttggcggtaacgtcccgaacgttgtcctcgagtgcaccggagccgagccctgcattaagctcggagtggatgccatcgcccctggtggacgctttgtccaggttggtaacgccgccggtcccgtcagcttcccgatcaccgttttcgctatgaaggagctcaccctcttcggcagcttccgttacggctttaacgactacaagaccgccgtgggcatctttgacaccaactaccagaacggacgtgagaacgcccctatcgattttgagcagctgattacccaccgttacaagtttaaggacgccattgaggcctacgacctcgtccgcgctggcaagggagccgtcaagtgcctcatcgatggtcccgagtgacatatgagttatgagatccgaaagtgaaccttgtcctaacccgacagcgaatggcgggagggggcgggctaaaagatcgtattacatagtatttttcccctactctttgtgtttgtcttttttttttttttgaacgcattcaagccacttgtctgggtttacttgtttgtttgcttgcttgcttgcttgcttgcctgcttcttggtcagacggcccaaaaaagggaaaaaattcattcatggcacagataagaaaaagaaaaagtttgtcgaccaccgtcatcagaaagcaagagaagagaaacactcgcgctcacattctcgctcgcgtaagaatcttagccacgcatacgaagtaatttgtccatctggcgaatctttacatgagcgttttcaagctggagcgtgagatcatacctttcttgatcgtaatgttccaaccttgcataggcctcgttgcgatccactagcaatgcgtcgtactcccgttgcaactgcgccatcgcctcattgtgacgtgagttcagattcttctcgagaccttcgagcgctgctaatttcgcctgacgctccttcttttgtgcttccatgacacgccgcttcaccgtgcgttccacttcttcctcagacatgcccttggctgcctcgacctgctcggtaaaacgggccccagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatacatggtcgacctgcaggaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaagcttgaggtctgtcgataatccacttttccattgattttccaggtttcgttaactcatgccactgagcaaaacttcggtctttcctaacaaaagctctcctcacaaagcatggcgcggcaacggacgtgtcctcatactccactgccacacaaggtcgataaactaagctcctcacaaatagaggagaattccactgacaactgaaaacaatgtatgagagacgatcaccactggagcggcgcggcggttgggcgcggaggtcggcagcaaaaacaagcgactcgccgagcaaacccgaatcagccttcagacggtcgtgcctaacaacacgccgttctaccccgccttcttcgcgccccttcgcgtccaagcatccttcaagtttatctctctagttcaacttcaagaagaacaacaccaccaacaccatgatgcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgtggacggtgccgacaggtgcttctcgatctgcatcctgggatcaaagccatagtgaaggacagtgatggacagccgacggcagttgggattcgtgaattgctgccctctggttatgtgtgggagggctaacacgtgctccgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgaattcccggggtac

SEQ ID NO: 19 - DNA, 7628 bp, Artificial Sequence
  vector pCL0135
ctcttatctgcctcgcgccgttgaccgccgcttgactcttggcgcttgccgctcgcatcctgcctcgctcgcgcaggcgggcgggcgagtgggtgggtccgcagccttccgcgctcgcccgctagctcgctcgcgccgtgctgcagccagcagggcagcaccgcacggcaggcaggtcccggcgcggatcgatcgatccatcgatccatcgatccatcgatcgtgcggtcaaaaagaaaggaagaagaaaggaaaaagaaaggcgtgcgcacccgagtgcgcgctgagcgcccgctcgcggtcccgcggagcctccgcgttagtccccgccccgcgccgcgcagtcccccgggaggcatcgcgcacctctcgccgccccctcgcgcctcgccgattccccgcctccccttttccgcttcttcgccgcctccgctcgcggccgcgtcgcccgcgccccgctccctatctgctccccaggggggcactccgcaccttttgcgcccgctgccgccgccgcggccgccccgccgccctggtttcccccgcgagcgcggccgcgtcgccgcgcaaagactcgccgcgtgccgccccgagcaacgggtggcggcggcgcggcggcgggcggggcgcggcggcgcgtaggcggggctaggcgccggctaggcgaaacgccgcccccgggcgccgccgccgcccgctccagagcagtcgccgcgccagaccgccaacgcagagaccgagaccgaggtacgtcgcgcccgagcacgccgcgacgcgcggcagggacgaggagcacgacgccgcgccgcgccgcgcggggggggggagggagaggcaggacgcgggagcgagcgtgcatgtttccgcgcgagacgacgccgcgcgcgctggagaggagataaggcgcttggatcgcgagagggccagccaggctggaggcgaaaatgggtggagaggatagtatcttgcgtgcttggacgaggagactgacgaggaggacggatacgtcgatgatgatgtgcacagagaagaagcagttcgaaagcgactactagcaagcaagggatccatgactactacgccgtttgacgctcccgacaagctctttcttggcttcgatctctccacccagcagcttaagattatcgtcactgacgagaacctcgccgctctcaagacctacaacgtcgagtttgatagcattaactccagcgtccagaagggtgtgatcgccattaacgatgagatcagcaagggagccatcatcagcccggtctacatgtggctcgacgctctcgatcacgtcttcgaggatatgaagaaggacggtttcccctttaacaaggtggtcggaatctccggctcgtgccagcagcacggttcggtctactggtcgcgcactgctgagaaggttctctccgagcttgacgccgagtcctccctctcgtcccagatgcgctccgcctttactttcaagcacgcccccaactggcaggaccactcgaccggcaaggagctcgaggagtttgagcgcgtcatcggcgccgacgccctcgctgacatctccggtagccgcgcccactaccgctttactggccttcagattcgcaagctctcgacccgttttaagcccgagaagtacaaccgcacggcccgcatttccctggtctccagcttcgtcgcttccgtccttctgggtcgcattacgtccatcgaggaggctgacgcttgcggcatgaacctctacgacatcgagaagcgcgagttcaacgaggagcttctcgccattgcggctggtgtccaccccgagctggacggtgtcgagcaggacggtgagatctaccgcgccggtattaacgagctcaagcgtaagctcggccctgtcaagcccatcacctacgagtccgagggagacatcgcctcctacttcgtcacccgctacggttttaaccctgactgcaagatctactcgtttactggagacaacctcgccaccatcatctcccttcctcttgccccgaacgacgccctcatcagccttggcacctccaccaccgtgcttatcatcaccaagaactacgccccgtcgtcccagtaccacctctttaagcacccgacgatgcccgaccactacatgggaatgatttgctactgcaacggctccctcgcccgtgagaaggttcgcgacgaggttaacgagaagtttaacgtcgaggacaagaagtcgtgggacaagtttaacgagatcctcgacaagagcaccgattttaacaacaagctcggcatctacttcccgctcggagagattgtccctaacgctgcggcccagattaagcgctcggtccttaactcgaagaacgagatcgtcgacgtcgagctcggagataagaactggcagcctgaggacgatgtgagcagcattgttgagtcccagaccctttcgtgccgcctccgcacgggcccgatgctctccaagtccggtgattcctccgcttcgtcgtccgcctcgccccagcccgagggagatggcacggacctccacaaggtttaccaggacctcgttaagaagttcggcgacctcttcaccgatggtaagaagcagacttttgagtccctcaccgcccgccccaaccgctgctactacgtcggtggcgccagcaacaacggctcgatcatcctcaagatgggcagcattctcgcccctgtgaacggtaactacaaggtcgatatcccgaacgcgtgcgcccttggcggagcttacaaggcgtcgtggagctacgagtgcgaggccaagaaggagtggattggctacgatcagtacattaaccgccttttcgaggtgtccgatgagatgaacagctttgaggtcaaggacaagtggctcgagtacgctaacggcgtgggcatgctcgccaagatggagtccgagctcaagcactgacatatgagttatgagatccgaaagtgaaccttgtcctaacccgacagcgaatggcgggagggggcgggctaaaagatcgtattacatagtatttttcccctactctttgtgtttgtcttttttttttttttgaacgcattcaagccacttgtctgggtttacttgtttgtttgcttgcttgcttgcttgcttgcctgcttcttggtcagacggcccaaaaaagggaaaaaattcattcatggcacagataagaaaaagaaaaagtttgtcgaccaccgtcatcagaaagcaagagaagagaaacactcgcgctcacattctcgctcgcgtaagaatcttagccacgcatacgaagtaatttgtccatctggcgaatctttacatgagcgttttcaagctggagcgtgagatcatacctttcttgatcgtaatgttccaaccttgcataggcctcgttgcgatccgctagcaatgcgtcgtactcccgttgcaactgcgccatcgcctcattgtgacgtgagttcagattcttctcgagaccttcgagcgctgctaatttcgcctgacgctccttcttttgtgcttccatgacacgccgcttcaccgtgcgttccacttcttcctcagacatgcccttggctgcctcgacctgctcggtaaaacgggccccagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatacatggtcgacctgcaggaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaagcttgaggtctgtcgataatccacttttccattgattttccaggtttcgttaactcatgccactgagcaaaacttcggtctttcctaacaaaagctctcctcacaaagcatggcgcggcaacggacgtgtcctcatactccactgccacacaaggtcgataaactaagctcctcacaaatagaggagaattccactgacaactgaaaacaatgtatgagagacgatcaccactggagcggcgcggcggttgggcgcggaggtcggcagcaaaaacaagcgactcgccgagcaaacccgaatcagccttcagacggtcgtgcctaacaacacgccgttctaccccgccttcttcgcgccccttcgcgtccaagcatccttcaagtttatctctctagttcaacttcaagaagaacaacaccaccaacaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgaattcccggggtac

SEQ ID NO: 20 - DNA, 7241 bp, Artificial Sequence
  vector pCL0136
ctcttatctgcctcgcgccgttgaccgccgcttgactcttggcgcttgccgctcgcatcctgcctcgctcgcgcaggcgggcgggcgagtgggtgggtccgcagccttccgcgctcgcccgctagctcgctcgcgccgtgctgcagccagcagggcagcaccgcacggcaggcaggtcccggcgcggatcgatcgatccatcgatccatcgatccatcgatcgtgcggtcaaaaagaaaggaagaagaaaggaaaaagaaaggcgtgcgcacccgagtgcgcgctgagcgcccgctcgcggtcccgcggagcctccgcgttagtccccgccccgcgccgcgcagtcccccgggaggcatcgcgcacctctcgccgccccctcgcgcctcgccgattccccgcctccccttttccgcttcttcgccgcctccgctcgcggccgcgtcgcccgcgccccgctccctatctgctccccaggggggcactccgcaccttttgcgcccgctgccgccgccgcggccgccccgccgccctggtttcccccgcgagcgcggccgcgtcgccgcgcaaagactcgccgcgtgccgccccgagcaacgggtggcggcggcgcggcggcgggcggggcgcggcggcgcgtaggcggggctaggcgccggctaggcgaaacgccgcccccgggcgccgccgccgcccgctccagagcagtcgccgcgccagaccgccaacgcagagaccgagaccgaggtacgtcgcgcccgagcacgccgcgacgcgcggcagggacgaggagcacgacgccgcgccgcgccgcgcggggggggggagggagaggcaggacgcgggagcgagcgtgcatgtttccgcgcgagacgacgccgcgcgcgctggagaggagataaggcgcttggatcgcgagagggccagccaggctggaggcgaaaatgggtggagaggatagtatcttgcgtgcttggacgaggagactgacgaggaggacggatacgtcgatgatgatgtgcacagagaagaagcagttcgaaagcgactactagcaagcaagggatccatgaagaccgtcgccggcatcgatcttggaacccagtccatgaaggttgtcatttacgactacgagaagaaggagatcatcgagtccgcctcgtgccctatggagctcattagcgagtcggacggaacccgcgagcagacgactgagtggtttgacaagggtctcgaggtgtgctttggaaagctctccgctgataacaagaagaccattgaggcgattggcatctccggccagctccacggcttcgtccctctcgatgcgaacggaaaggcgctctacaacatcaagctctggtgcgacaccgccactgtggaggagtgcaagatcattactgacgccgccggcggcgacaaggctgtcatcgacgcgctcggcaacctcatgctcaccggattcaccgccccgaagattctctggctcaagcgcaacaagcccgaggcctttgctaacctcaagtacattatgctgccccacgattacctcaactggaagctgactggagactacgtcatggagtacggcgacgcctccggcaccgccctttttgattcgaagaaccgctgctggtcgaagaagatttgcgacattattgatcctaagctgctcgaccttctccctaagctcattgagccctcggcccccgccggtaaggtcaacgacgaggccgccaaggcgtacggcattcccgccggaatccccgtttccgctggcggcggtgataacatgatgggtgcggtcggtactggcaccgtcgctgacggattcctcacgatgagcatgggcacctccggaactctttacggctactcggacaagcctatttccgacccggctaacggcctcagcggcttctgcagctccacgggcggctggcttcccctcctttgcaccatgaactgcaccgtcgccaccgagttcgtccgcaacctttttcagatggatatcaaggagctgaacgtcgaggctgctaagtccccctgcggcagcgagggcgttcttgtcattcctttcttcaacggcgagcgcaccccgaacctccccaacggccgcgcctcgattaccggcctcacctccgcgaacacgtcccgcgccaacatcgctcgcgcctcctttgagtcggccgtctttgccatgcgcggtggcctcgatgcgtttcgtaagctcggattccagcccaaggagattcgcctcatcggcggtggttcgaagtccgacctctggcgccagatcgctgctgacattatgaaccttcccatccgtgtcccccttctcgaggaggccgccgccctcggcggagctgtccaggccctttggtgccttaagaaccagtccggtaagtgcgacatcgtcgagctttgcaaggagcatatcaagattgacgagtccaagaacgccaacccgattgccgagaacgtcgccgtgtacgataaggcctacgatgagtactgcaaggtcgttaacacgctcagccctctgtacgcctaacatatgagttatgagatccgaaagtgaaccttgtcctaacccgacagcgaatggcgggagggggcgggctaaaagatcgtattacatagtatttttcccctactctttgtgtttgtcttttttttttttttgaacgcattcaagccacttgtctgggtttacttgtttgtttgcttgcttgcttgcttgcttgcctgcttcttggtcagacggcccaaaaaagggaaaaaattcattcatggcacagataagaaaaagaaaaagtttgtcgaccaccgtcatcagaaagcaagagaagagaaacactcgcgctcacattctcgctcgcgtaagaatcttagccacgcatacgaagtaatttgtccatctggcgaatctttacatgagcgttttcaagctggagcgtgagatcatacctttcttgatcgtaatgttccaaccttgcataggcctcgttgcgatccgctagcaatgcgtcgtactcccgttgcaactgcgccatcgcctcattgtgacgtgagttcagattcttctcgagaccttcgagcgctgctaatttcgcctgacgctccttcttttgtgcttccatgacacgccgcttcaccgtgcgttccacttcttcctcagacatgcccttggctgcctcgacctgctcggtaaaacgggccccagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatacatggtcgacctgcaggaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaagcttgaggtctgtcgataatccacttttccattgattttccaggtttcgttaactcatgccactgagcaaaacttcggtctttcctaacaaaagctctcctcacaaagcatggcgcggcaacggacgtgtcctcatactccactgccacacaaggtcgataaactaagctcctcacaaatagaggagaattccactgacaactgaaaacaatgtatgagagacgatcaccactggagcggcgcggcggttgggcgcggaggtcggcagcaaaaacaagcgactcgccgagcaaacccgaatcagccttcagacggtcgtgcctaacaacacgccgttctaccccgccttcttcgcgccccttcgcgtccaagcatccttcaagtttatctctctagttcaacttcaagaagaacaacaccaccaacaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgaattcccggggtac

SEQ ID NO: 21 - DNA, 957 bp, Pichia stipitis
  xylose reductase (X59465), codon optimized
atgccctccattaagctcaactccggttacgatatgcccgccgtcggttttggttgctggaaggtggacgtcgacacttgctcggagcagatttaccgcgccattaagaccggataccgcctctttgacggtgccgaggactacgccaacgagaagctggtcggagccggcgtcaagaaggccattgatgagggaattgtcaagcgcgaggacctctttctcacctccaagctctggaacaactaccaccaccccgataacgtcgagaaggctcttaaccgtaccctcagcgatctccaggtcgactacgtcgatctttttcttattcacttccctgtcacgttcaagtttgtccctcttgaggagaagtacccccccggattctactgcggaaagggcgataactttgactacgaggacgttcctattctggagacttggaaggctctcgagaagctcgtcaaggccggcaagattcgcagcatcggcgtcagcaactttcctggagctctcctcctggacctccttcgcggagccaccatcaagccttcggttcttcaggtcgagcaccatccttaccttcagcagccccgtctcatcgagtttgcccagtcccgcggtattgccgtcacggcctacagctccttcggccctcagtcctttgtcgagctcaaccagggtcgcgcccttaacaccagccccctcttcgagaacgagaccattaaggccatcgctgctaagcacggtaagtcccccgcccaggtcctcctccgttggagctcgcagcgcggaatcgccatcatccctaagagcaacaccgtccctcgccttcttgagaacaaggatgtcaactccttcgacctcgatgagcaggatttcgccgacattgccaagctcgatattaacctccgcttcaacgacccctgggactgggataagatccctatctttgtctaa

SEQ ID NO: 22 - DNA, 1872 bp, Pichia stipitis
  xylulose kinase (AF127802), codon optimized
atgactactacgccgtttgacgctcccgacaagctctttcttggcttcgatctctccacccagcagcttaagattatcgtcactgacgagaacctcgccgctctcaagacctacaacgtcgagtttgatagcattaactccagcgtccagaagggtgtgatcgccattaacgatgagatcagcaagggagccatcatcagcccggtctacatgtggctcgacgctctcgatcacgtcttcgaggatatgaagaaggacggtttcccctttaacaaggtggtcggaatctccggctcgtgccagcagcacggttcggtctactggtcgcgcactgctgagaaggttctctccgagcttgacgccgagtcctccctctcgtcccagatgcgctccgcctttactttcaagcacgcccccaactggcaggaccactcgaccggcaaggagctcgaggagtttgagcgcgtcatcggcgccgacgccctcgctgacatctccggtagccgcgcccactaccgctttactggccttcagattcgcaagctctcgacccgttttaagcccgagaagtacaaccgcacggcccgcatttccctggtctccagcttcgtcgcttccgtccttctgggtcgcattacgtccatcgaggaggctgacgcttgcggcatgaacctctacgacatcgagaagcgcgagttcaacgaggagcttctcgccattgcggctggtgtccaccccgagctggacggtgtcgagcaggacggtgagatctaccgcgccggtattaacgagctcaagcgtaagctcggccctgtcaagcccatcacctacgagtccgagggagacatcgcctcctacttcgtcacccgctacggttttaaccctgactgcaagatctactcgtttactggagacaacctcgccaccatcatctcccttcctcttgccccgaacgacgccctcatcagccttggcacctccaccaccgtgcttatcatcaccaagaactacgccccgtcgtcccagtaccacctctttaagcacccgacgatgcccgaccactacatgggaatgatttgctactgcaacggctccctcgcccgtgagaaggttcgcgacgaggttaacgagaagtttaacgtcgaggacaagaagtcgtgggacaagtttaacgagatcctcgacaagagcaccgattttaacaacaagctcggcatctacttcccgctcggagagattgtccctaacgctgcggcccagattaagcgctcggtccttaactcgaagaacgagatcgtcgacgtcgagctcggagataagaactggcagcctgaggacgatgtgagcagcattgttgagtcccagaccctttcgtgccgcctccgcacgggcccgatgctctccaagtccggtgattcctccgcttcgtcgtccgcctcgccccagcccgagggagatggcacggacctccacaaggtttaccaggacctcgttaagaagttcggcgacctcttcaccgatggtaagaagcagacttttgagtccctcaccgcccgccccaaccgctgctactacgtcggtggcgccagcaacaacggctcgatcatcctcaagatgggcagcattctcgcccctgtgaacggtaactacaaggtcgatatcccgaacgcgtgcgcccttggcggagcttacaaggcgtcgtggagctacgagtgcgaggccaagaaggagtggattggctacgatcagtacattaaccgccttttcgaggtgtccgatgagatgaacagctttgaggtcaaggacaagtggctcgagtacgctaacggcgtgggcatgctcgccaagatggagtccgagctcaagcactga

SEQ ID NO: 23 - DNA, 1092 bp, Pichia stipitis
  Xylitol dehydrogenase (X55392), codon optimized
atgaccgccaacccgagcctcgtccttaacaagatcgacgatatttccttcgagacctacgacgcccccgagatcagcgagcccaccgatgtcctcgtccaggttaagaagaccggcatctgcggttccgatattcacttttacgctcacggacgcattggaaactttgtcctcactaagcctatggttctgggtcacgagtccgccggtactgtcgttcaggtgggaaagggtgttacgtcgcttaaggtcggagacaacgttgccatcgagcccggcatccccagccgcttttccgatgagtacaagtccggtcactacaacctctgcccccacatggctttcgccgccacccccaactccaaggagggcgagcctaacccccccggcaccctctgcaagtactttaagtcccccgaggattttctcgtcaagctccccgaccacgtctcgcttgagctgggcgccctcgtcgagcccctgtccgtcggagttcacgccagcaagctcggtagcgttgcctttggcgactacgtggccgtttttggcgcgggtcctgtcggccttctcgccgccgctgtggccaagacctttggagccaagggcgttatcgtcgtcgacatttttgacaacaagctcaagatggctaaggatattggcgccgctactcatacctttaactccaagaccggcggttccgaggagcttatcaaggcctttggcggtaacgtcccgaacgttgtcctcgagtgcaccggagccgagccctgcattaagctcggagtggatgccatcgcccctggtggacgctttgtccaggttggtaacgccgccggtcccgtcagcttcccgatcaccgttttcgctatgaaggagctcaccctcttcggcagcttccgttacggctttaacgactacaagaccgccgtgggcatctttgacaccaactaccagaacggacgtgagaacgcccctatcgattttgagcagctgattacccaccgttacaagtttaaggacgccattgaggcctacgacctcgtccgcgctggcaagggagccgtcaagtgcctcatcgatggtcccgagtga

SEQ ID NO: 24 - protein, 18 aa, Artificial Sequence
  synthetic peptide
RLRKLPIDHPDSLEELRD

SEQ ID NO: 25 - protein, 15 aa, Artificial Sequence
  synthetic peptide
ETKGLTLEEIEAKCL